SEQUENCE LISTING (59 sequences)

SEQ ID NO: 1 - protein, 95 aa, synthetic construct
  REGION 1..95: note = Synthetic: 1-A4
QAGGSLRLSCAASGYIFSDTYMGWYRQAPGKEREFVAGINGGGTTNYADSVKGRFTISRDNAKNTVYLQMNSLKPEDTAVYYCAVYYGSFSWSLL

SEQ ID NO: 2 - protein, 114 aa, synthetic construct
  REGION 1..114: note = Synthetic: 1-D5
AGGSLRLSCAASGNISYSYGMGWYRQAPGKEREFVAGITFGGSTYYADSVKGRFTISRDNAKNTVYLQMNSLKPEDTAVYYCAVYTRHVDTTFARHWYWGQGTQVTVSSLEHHH

SEQ ID NO: 3 - protein, 109 aa, synthetic construct
  REGION 1..109: note = Synthetic: I-D8
AGGSLRLSCAASGNIFYGQPMGWYRQAPGKEREFVAGIGRGGSTYYADSVKGRFTISRDNAKNTVYLQMNSLKPEDTAVYYCAVLSQYPYRHTYWGQGTQVTVSSLEHH

SEQ ID NO: 4 - protein, 113 aa, synthetic construct
  REGION 1..113: note = Synthetic: I-H4
QAGGSLRLSCAASGTISTYGMGWYRQAPGKEREFVAGIATGGTTYYADSVKGRFTISRDNAKNTVYLQMNSLKPEDTAVYYCAALDKYARHYVYWGQGTQVTVSSLEHHHHHH

SEQ ID NO: 5 - protein, 112 aa, synthetic construct
  REGION 1..112: note = Synthetic: II-B1
QAGGSLRLSCAASGTIFYRYSMGWYRQAPGKEREFVAGITEGSNTYYADSVKGRFTISRDNAKNTVYLQMNSLKPEDTAVYYCAVVQRVDLTYWGQGTQVTVSSLEHHHHHH

SEQ ID NO: 6 - protein, 118 aa, synthetic construct
  REGION 1..118: note = Synthetic: II-B2
QAGGSLRLSCAASGNIFRVIGMGWYRQAPGKEREFVAGIGSGSSTYYADSVKGRFTISRDNAKNTVYLQMNSLKPEDTAVYYCAAFTYPYYDQTKLLPYWGQGTQVTVSSLEHHHHHH

SEQ ID NO: 7 - protein, 118 aa, synthetic construct
  REGION 1..118: note = Synthetic: II-C10
QAGGSLRLSCAASGTIFPRANMGWYRQAPGKEREFVAGITLGGTTYYADSVKGRFTISRDNAKNTVYLQMNSLKPEDTAVYYCAVVYKTYRYQEILYYYWGQGTQVTVSSLEHHHHHH

SEQ ID NO: 8 - protein, 118 aa, synthetic construct
  REGION 1..118: note = Synthetic: IG11
QAGGSLRLSCAASGNIFRSSAMGWYRQAPGKEREFVAGITYGTNTYYADSVKGRFTISRDNAKNTVYLQMNSLKPEDTAVYYCAASTPARDALSQDLWYWGQGTQVTVSSLEHHHHHH

SEQ ID NO: 9 - protein, 113 aa, synthetic construct
  REGION 1..113: note = Synthetic: IG12
QAGGSLRLSCAASGSIFPYSFMGWYRQAPGKEREFVAGISPGSNTYYADSVKGRFTISRDNAKNTVYLQMNSLKPEDTAVYYCAVVSDYQSHYYWGQGTQVTVSSLEHHHHHH

SEQ ID NO: 10 - protein, 118 aa, synthetic construct
  REGION 1..118: note = Synthetic: IIG12
QAGGSLRLSCAASGTIFFRSYMGWYRQAPGKEREFVAGIGYGGNTYYADSVKGRFTISRDNAKNTVYLQMNSLKPEDTAVYYCAVYNRDYISPYGAHTYWGQGTQVTVSSLEHHHHHH

SEQ ID NO: 11-13 - skipped (3 entries)

SEQ ID NO: 14 - protein, 7 aa, synthetic construct
  REGION 1..7: note = Synthetic: 1-D5 CDR 1
NISYSYG

SEQ ID NO: 15 - protein, 7 aa, synthetic construct
  REGION 1..7: note = Synthetic: 1-D5 CDR2
GITFGGS

SEQ ID NO: 16 - protein, 14 aa, synthetic construct
  REGION 1..14: note = Synthetic: 1-D5 CDR3
VYTRHVDTTFARHW

SEQ ID NO: 17 - protein, 7 aa, synthetic construct
  REGION 1..7: note = Synthetic: I-D8 CDR 1
NIFYGQP

SEQ ID NO: 18 - protein, 7 aa, synthetic construct
  REGION 1..7: note = Synthetic: I-D8 CDR2
GIGRGGS

SEQ ID NO: 19 - protein, 10 aa, synthetic construct
  REGION 1..10: note = Synthetic: I-D8 CDR3
VLSQYPYRHT

SEQ ID NO: 20 - protein, 6 aa, synthetic construct
  REGION 1..6: note = Synthetic: I-H4 CDR 1
TISTYG

SEQ ID NO: 21 - protein, 7 aa, synthetic construct
  REGION 1..7: note = Synthetic: I-H4 CDR2
GIATGGT

SEQ ID NO: 22 - protein, 10 aa, synthetic construct
  REGION 1..10: note = Synthetic: I-H4 CDR3
ALDKYARHYV

SEQ ID NO: 23 - protein, 7 aa, synthetic construct
  REGION 1..7: note = Synthetic: II-B1 CDR 1
TIFYRYS

SEQ ID NO: 24 - protein, 7 aa, synthetic construct
  REGION 1..7: note = Synthetic: II-B1 CDR2
GITEGSN

SEQ ID NO: 25 - protein, 8 aa, synthetic construct
  REGION 1..8: note = Synthetic: II-B1 CDR3
VVQRVDLT

SEQ ID NO: 26 - protein, 7 aa, synthetic construct
  REGION 1..7: note = Synthetic: II-B2 CDR 1
NIFRVIG

SEQ ID NO: 27 - protein, 7 aa, synthetic construct
  REGION 1..7: note = Synthetic: II-B2 CDR2
GIGSGSS

SEQ ID NO: 28 - protein, 14 aa, synthetic construct
  REGION 1..14: note = Synthetic: II-B2 CDR3
AFTYPYYDQTKLLP

SEQ ID NO: 29 - protein, 7 aa, synthetic construct
  REGION 1..7: note = Synthetic: II-C10 CDR 1
TIFPRAN

SEQ ID NO: 30 - protein, 7 aa, synthetic construct
  REGION 1..7: note = Synthetic: II-C10 CDR2
GITLGGT

SEQ ID NO: 31 - protein, 14 aa, synthetic construct
  REGION 1..14: note = Synthetic: II-C10 CDR3
VVYKTYRYQEILYY

SEQ ID NO: 32 - protein, 7 aa, synthetic construct
  REGION 1..7: note = Synthetic: IG11 CDR 1
NIFRSSA

SEQ ID NO: 33 - protein, 7 aa, synthetic construct
  REGION 1..7: note = Synthetic: IG11 CDR2
GITYGTN

SEQ ID NO: 34 - protein, 14 aa, synthetic construct
  REGION 1..14: note = Synthetic: IG11 CDR3
ASTPARDALSQDLW

SEQ ID NO: 35 - protein, 7 aa, synthetic construct
  REGION 1..7: note = Synthetic: IG12 CDR 1
SIFPYSF

SEQ ID NO: 36 - protein, 7 aa, synthetic construct
  REGION 1..7: note = Synthetic: IG12 CDR2
GISPGSN

SEQ ID NO: 37 - protein, 10 aa, synthetic construct
  REGION 1..10: note = Synthetic: IG12 CDR3
VVSDYQSHYY

SEQ ID NO: 38 - protein, 7 aa, synthetic construct
  REGION 1..7: note = Synthetic: IIG12 CDR 1
TIFFRSY

SEQ ID NO: 39 - protein, 7 aa, synthetic construct
  REGION 1..7: note = Synthetic: IIG12 CDR2
GIGYGGN

SEQ ID NO: 40 - protein, 14 aa, synthetic construct
  REGION 1..14: note = Synthetic: IIG12 CDR3
VYNRDYISPYGAHT

SEQ ID NO: 41 - protein, 9 aa, synthetic construct
  REGION 1..9: note = Synthetic: 1-A4
DGTLAVPFK

SEQ ID NO: 42 - DNA, 351 bp, synthetic construct
  misc_feature 1..351: note = Synthetic: 1-D5
  source 1..351: mol_type = other DNA
gcgggcggcagcctgcgcctgagctgcgcggcgagcggcaatatttcttactcttacggtatgggctggtatcgccaggcgccgggcaaagaacgcgaatttgttgccggtattactttcggtggtagtacctattatgcggatagcgtgaaaggccgctttaccattagccgcgataacgcgaaaaacaccgtgtatctgcagatgaacagcctgaaaccggaagataccgcggtgtattattgcgcggtttacactcgtcacgttgacactactttcgctcgtcattggtattggggccagggcacccaggtgaccgtgagcagcctcgagcaccaccaccaccaccac

SEQ ID NO: 43 - DNA, 339 bp, synthetic construct
  misc_feature 1..339: note = Synthetic: I-D8
  source 1..339: mol_type = other DNA
gcgggcggcagcctgcgcctgagctgcgcggcgagcggcaatattttttacggtcagccgatgggctggtatcgccaggcgccgggcaaagaacgcgaatttgttgccggtattggtcgtggtggtagtacctattatgcggatagcgtgaaaggccgctttaccattagccgcgataacgcgaaaaacaccgtgtatctgcagatgaacagcctgaaaccggaagataccgcggtgtattattgcgcggttctgtctcagtacccgtaccgtcatacttattggggccagggcacccaggtgaccgtgagcagcctcgagcaccaccaccaccaccac

SEQ ID NO: 44 - DNA, 330 bp, synthetic construct
  misc_feature 1..330: note = Synthetic: I-H4
  source 1..330: mol_type = other DNA
ggcagcctgcgcctgagctgcgcggcgagcggcactatttctacttacggtatgggctggtatcgccaggcgccgggcaaagaacgcgaatttgttgccggtattgctactggtggtactacctattatgcggatagcgtgaaaggccgctttaccattagccgcgataacgcgaaaaacaccgtgtatctgcagatgaacagcctgaaaccggaagataccgcggtgtattattgcgcggctctggacaaatacgctcgtcattatgtttattggggccagggcacccaggtgaccgtgagcagcctcgagcaccaccaccaccaccac

SEQ ID NO: 45 - DNA, 333 bp, synthetic construct
  misc_feature 1..333: note = Synthetic: II-B1
  source 1..333: mol_type = other DNA
gcgggcggcagcctgcgcctgagctgcgcggcgagcggcactattttttaccgttactctatgggctggtatcgccaggcgccgggcaaagaacgcgaatttgttgccggtattactgaaggtagtaatacctattatgcggatagcgtgaaaggccgctttaccattagccgcgataacgcgaaaaacaccgtgtatctgcagatgaacagcctgaaaccggaagataccgcggtgtattattgcgcggttgttcagcgtgttgaccttacttattggggccagggcacccaggtgaccgtgagcagcctcgagcaccaccaccaccaccac

SEQ ID NO: 46 - DNA, 351 bp, synthetic construct
  misc_feature 1..351: note = Synthetic: II-B2
  source 1..351: mol_type = other DNA
gcgggcggcagcctgcgcctgagctgcgcggcgagcggcaatatttttcgtgttatcggtatgggctggtatcgccaggcgccgggcaaagaacgcgaatttgttgccggtattggttctggtagtagtacctattatgcggatagcgtgaaaggccgctttaccattagccgcgataacgcgaaaaacaccgtgtatctgcagatgaacagcctgaaaccggaagataccgcggtgtattattgcgcggctttcacttacccgtactacgaccagactaaactgcttccgtattggggccagggcacccaggtgaccgtgagcagcctcgagcaccaccaccaccaccac

SEQ ID NO: 47 - DNA, 351 bp, synthetic construct
  misc_feature 1..351: note = Synthetic: II-C10
  source 1..351: mol_type = other DNA
gcgggcggcagcctgcgcctgagctgcgcggcgagcggcactatttttccgcgtgctaacatgggctggtatcgccaggcgccgggcaaagaacgcgaatttgttgccggtattactctgggtggtactacctattatgcggatagcgtgaaaggccgctttaccattagccgcgataacgcgaaaaacaccgtgtatctgcagatgaacagcctgaaaccggaagataccgcggtgtattattgcgcggttgtttacaaaacttaccgttaccaggaaatcctgtattactattggggccagggcacccaggtgaccgtgagcagcctcgagcaccaccaccaccaccac

SEQ ID NO: 48 - DNA, 351 bp, synthetic construct
  misc_feature 1..351: note = Synthetic: IG11
  source 1..351: mol_type = other DNA
gcgggcggcagcctgcgcctgagctgcgcggcgagcggcaatatttttcgttcttctgctatgggctggtatcgccaggcgccgggcaaagaacgcgaatttgttgccggtattacttacggtactaatacctattatgcggatagcgtgaaaggccgctttaccattagccgcgataacgcgaaaaacaccgtgtatctgcagatgaacagcctgaaaccggaagataccgcggtgtattattgcgcggcttctactccggctcgtgacgctctgtctcaggacctttggtattggggccagggcacccaggtgaccgtgagcagcctcgagcaccaccaccaccaccac

SEQ ID NO: 49 - DNA, 309 bp, synthetic construct
  misc_feature 1..309: note = Synthetic: IG12
  source 1..309: mol_type = other DNA
gcggcgagcggctctatttttccgtactctttcatgggctggtatcgccaggcgccgggcaaagaacgcgaatttgttgccggtattagtccgggtagtaatacctattatgcggatagcgtgaaaggccgctttaccattagccgcgataacgcgaaaaacaccgtgtatctgcagatgaacagcctgaaaccggaagataccgcggtgtattattgcgcggttgtttctgactaccagtctcattactattggggccagggcacccaggtgaccgtgagcagcctcgagcaccaccaccaccaccac

SEQ ID NO: 50 - DNA, 351 bp, synthetic construct
  misc_feature 1..351: note = Synthetic: IIG12
  source 1..351: mol_type = other DNA
gcgggcggcagcctgcgcctgagctgcgcggcgagcggcactatttttttccgttcttacatgggctggtatcgccaggcgccgggcaaagaacgcgaatttgttgccggtattggttacggtggtaatacctattatgcggatagcgtgaaaggccgctttaccattagccgcgataacgcgaaaaacaccgtgtatctgcagatgaacagcctgaaaccggaagataccgcggtgtattattgcgcggtttacaaccgtgactacatctctccgtacggtgctcatacttattggggccagggcacccaggtgaccgtgagcagcctcgagcaccaccaccaccaccac

SEQ ID NO: 51 - protein, 20 aa, synthetic construct
  REGION 1..20: note = Synthetic: peptide linker
  VARIANT 6..20: note = "GGGGS" may be present or absent
GGGGSGGGGSGGGGSGGGGS

SEQ ID NO: 52 - protein, 8 aa, synthetic construct
  REGION 1..8: note = Synthetic: peptide linker
GGGGGGGG

SEQ ID NO: 53 - protein, 6 aa, synthetic construct
  REGION 1..6: note = Synthetic: peptide linker
GGGGGG

SEQ ID NO: 54 - protein, 15 aa, synthetic construct
  REGION 1..15: note = Synthetic: peptide linker
EAAAKEAAAKEAAAK

SEQ ID NO: 55 - protein, 15 aa, synthetic construct
  REGION 1..15: note = Synthetic: peptide linker
  VARIANT 6..15: note = "EAAAK" may be present or absent
EAAAKEAAAKEAAAK

SEQ ID NO: 56 - protein, 46 aa, synthetic construct
  REGION 1..46: note = Synthetic: peptide linker
AEAAAKEAAAKEAAAKEAAAKALEAEAAAKEAAAKEAAAKEAAAKA

SEQ ID NO: 57 - protein, 5 aa, synthetic construct
  REGION 1..5: note = Synthetic: peptide linker
PAPAP

SEQ ID NO: 58 - protein, 12 aa, synthetic construct
  REGION 1..12: note = Synthetic: peptide linker
AEAAAKEAAAKA

SEQ ID NO: 59 - protein, 68 aa, synthetic construct
  REGION 1..68: note = Synthetic: peptide linker
  VARIANT 21..68: note = "AP" may be present or absent
APAPAPAPAPAPAPAPAPAPAPAPAPAPAPAPAPAPAPAPAPAPAPAPAPAPAPAPAPAPAPAPAPAP

(19) United States
(12) Patent Application Publication
(10) Pub. No.: US 2024/0409664 A1
(43) Pub. Date: Dec. 12, 2024

(54) ANTI-PMEPA-1 ANTIBODIES OR ANTIGEN BINDING FRAGMENTS THEREOF, COMPOSITIONS, AND USES THEREOF

(71) Applicant: President and Fellows of Harvard College, Cambridge, MA (US)

(72) Inventors: Harold Roderick Neely, Cambridge, MA (US); Irina Borisovna Mazo, Cambridge, MA (US); Ulrich H. Von Andrian, Cambridge, MA (US); Munir M. Mosaheb, Canbridge, MA (US)

(73) Assignee: President and Fellows of Harvard College, Cambridge, MA (US)

(21) Appl. No.: 18/668,561

(22) Filed: May 20, 2024

Related U.S. Application Data
(63) Continuation of application No. PCT/US2022/080450, filed on Nov. 23, 2022.
(60) Provisional application No. 63/282,589, filed on Nov. 23, 2021.

(51) Int. Cl. C07K 16/30; A61K 39/00

(52) U.S. Cl. CPC C07K 16/3069; A61K 2039/505; C07K 2317/569

(57) ABSTRACT

The technology described herein is directed to antibody or antigen binding fragments thereof, e.g., nanobody, to PMEPA-1 overexpressed in tumor vascular endothelial cells. The antibody or antigen binding fragments to PMEPA-1 can be used to target an agent, for example, that induces cell death, for example, immunogenic or non-immunogenic cancer cell death, and in methods of treating cancer.

### RELATED APPLICATIONS

This instant application is a continuation of International Application No. PCT/US2022/080450, filed May 23, 2023, which claims the benefit of priority to U.S. Provisional Application No. 63/282,589, filed on Nov. 23, 2021. The entire contents of each of the foregoing applications are expressly incorporated herein by reference.

### GOVERNMENT SUPPORT

This invention was made with government support under grants AI155865 and AI112521 awarded by the National Institutes of Health (NIH). The government has certain rights to this invention.

### REFERENCE TO ELECTRONIC SEQUENCE LISTING

The application contains a Sequence Listing which has been submitted electronically in .XML format and is hereby incorporated by reference in its entirety. Said .XML copy, created on May 17, 2024, is named “117823-32602.xml” and is 76,631 bytes in size. The sequence listing contained in this .XML file is part of the specification and is hereby incorporated by reference herein in its entirety.

### TECHNICAL FIELD

Described herein are compositions and methods related to targeting and treating cancer with anti-PMEPA-1 antibodies or antigen binding fragments thereof that bind PMEPA-1 in tumor vascular endothelial cells.

### BACKGROUND OF THE INVENTION

Recently, the American Cancer Society revealed that cancer has become the number one cause of death in 21 states and projected 1.7 million new cancer cases will arise in the U.S. for 2016 and 8.2 million cancer related deaths worldwide. Advances in immuno-oncology indicate that a cancer patient's immune system can be therapeutically harnessed to eliminate malignant tumors, providing long lasting responses in some cancers. However, despite this progress, current immunotherapy regimens have only shown efficacy in a subset of malignancies and/or a minority of patients. The high failure rate of cancer immunotherapy is inversely correlated with the presence of tumor-infiltrating T cells. The reason(s) for the paucity of T cells in so-called non-inflammatory tumors (which have a poor prognosis) are not well understood, but likely involve the inability of circulating tumor antigen-specific T cells to adhere to and emigrate from tumor microvessels into the surrounding tissue.

Recent clinical experience indicates that a cancer patient's immune system can be therapeutically harnessed to attack malignant tumors and induce long-lasting tumor regression (1). For example, treatment with anti-CTLA-4, an immune checkpoint inhibitor (CPI), results in tumor regression and long-term survival of a subset of patients (˜20%) with certain types of cancers, such as melanoma (2). Similarly, anti-PD1 antibodies have also been successful for some patients and are used as first-line therapy for melanoma, gastric cancer, hepatocellular carcinoma, head and neck squamous cell carcinoma and urothelial cancer (3). However, despite this progress, current immunotherapy regimens show efficacy only in a subset of malignancies and/or a minority of patients. The high failure rate of cancer immunotherapy is inversely correlated with the presence of tumor-infiltrating T lymphocytes (TILs). It has been documented in many studies that many immunotherapy resistant tumors lack T cells within the tumors and these cells instead accumulate in the tumor periphery, also a common observation by clinical pathologist (4-6).

The latest advances in cancer-immunotherapy have provided patients with a new type of treatment in which chimeric antigen receptor (CAR) T cells are generated to target malignant cells. However, CAR T cells have only limited clinical success for solid tumors (10, 11). This is probably due to the fact that blood-borne T cells are often unable to overcome the vascular barrier posed by the local microcirculation to access extravascular tumor cells. Moreover, even in settings where tumors are successfully targeted, the inherent genetic instability may allow tumor cells to acquire mutations resulting in resistance. Many tumors are also highly heterogeneous among and even within patients, so the efficacy of direct tumor targeting strategies can be highly variable.

The reason(s) for the paucity of T cells in so-called non-inflammatory tumors (which have a poor prognosis) are not well understood, but likely involve the inability of circulating tumor specific T cells to adhere to and emigrate from local microvessels into the surrounding tumor.

### SUMMARY OF THE INVENTION

A cancer patient's immune system can be therapeutically harnessed to eliminate malignant tumors. However, current immunotherapy regimens have shown efficacy only in a minority of malignancies. This high failure rate is inversely correlated with the presence of tumor-infiltrating T cells. The reasons for the paucity of T cells in so-called non-inflammatory (immunotherapy-resistant) tumors are poorly understood, but likely involve the inability of circulating T cells to adhere to and emigrate from tumor microvessels into surrounding tissue. The present invention provides antibodies or antigen binding fragments, in particular, nanobodies, for targeting a specific transmembrane molecule, PMEPA-1, that is upregulated in both murine and human tumor microvasculature and not in healthy tissues (e.g., non-tumor vascular endothelial cells). These antibodies or antigen binding fragments thereof, e.g., nanobodies, specific for PMEPA-1, a differentially-expressed transmembrane molecule, allow for tumor targeted treatment, via, for example, targeted CAR T cell therapy and other modes of targeted delivery of therapeutics. In another aspect, the present antibodies or antigen binding fragments thereof, e.g., a nanobody, to PMEPA-1, enable highly specific diagnostic imaging.

Accordingly, in one aspect, the invention comprises antibody or antigen-binding fragments thereof (e.g., nanobodies) for targeting PMEPA-1 and, thus, the intra-humoral microvasculature, which allow for methods to increase T cell recruitment into tumors so as to boost endogenous anti-tumor immunity and to synergize with other immuno-oncology approaches (see U.S. Provisional Application No. 63/282,565, filed Nov. 23, 2021 and entitled Compositions and Methods for Treating Cancer by Targeting Endothelial Cells having Upregulated Expression of Transmembrane Molecules, the entire contents of which are incorporated by reference herein).

The present disclosure provides an antibody or antigen binding fragment thereof, which specifically binds to Prostate Transmembrane Protein, Androgen Induced 1 (PMEPA-1).

In some embodiments, the antibody or antigen binding fragment thereof is selected from the group consisting of a monoclonal antibody, human antibody, a humanized antibody, a chimeric antibody, a recombinant antibody, a multispecific antibody, or an antigen-binding fragment thereof; wherein the antigen-binding fragment is 1) an Fv, Fab, F(ab′)2, Fab′, dsFv, scFv, or sc(Fv)2; or 2) a diabody, ScFv, SMIP, single chain antibody, affibody, avimer, or nanobody; or 3) a single domain antibody, and an antigen binding fragment of any of the foregoing.

In some embodiments, the antigen binding fragment is a nanobody.

In some embodiments, the antibody or antigen binding fragment thereof comprises: (1) a CDR1 sequence comprising the amino acid sequence of SEQ ID NO: 14, a CDR2 sequence comprising the amino acid sequence of SEQ ID NO: 15, and a CDR3 sequence comprising the amino acid sequence of SEQ ID NO: 16; or (2) a CDR1 sequence comprising the amino acid sequence of SEQ ID NO: 17, a CDR2 sequence comprising the amino acid sequence of SEQ ID NO: 18, and a CDR3 sequence comprising the amino acid sequence of SEQ ID NO: 19; or (3) a CDR1 sequence comprising the amino acid sequence of SEQ ID NO: 20, a CDR2 sequence comprising the amino acid sequence of SEQ ID NO: 21, and a CDR3 sequence comprising the amino acid sequence of SEQ ID NO: 22; or (4) a CDR1 sequence comprising the amino acid sequence of SEQ ID NO: 23, a CDR2 sequence comprising the amino acid sequence of SEQ ID NO: 24, and a CDR3 sequence comprising the amino acid sequence of SEQ ID NO: 25; or (5) a CDR1 sequence comprising the amino acid sequence of SEQ ID NO: 26, a CDR2 sequence comprising the amino acid sequence of SEQ ID NO: 27, and a CDR3 sequence comprising the amino acid sequence of SEQ ID NO: 28; or (6) a CDR1 sequence comprising the amino acid sequence of SEQ ID NO: 29, a CDR2 sequence comprising the amino acid sequence of SEQ ID NO: 30, and a CDR3 sequence comprising the amino acid sequence of SEQ ID NO: 31.

In some embodiments, the antibody or antigen binding fragment thereof comprises an amino acid sequence that has at least 90%, 91%, 92%, 93%, 94%, 95%, 96%, 97%, 98%, or 99% identity to, comprises, or consists of an amino acid sequence selected from SEQ ID NOs: 1-7. In some embodiments, the antibody or antigen binding fragment thereof comprises the amino acid sequence of SEQ ID NO: 5. In some embodiments, the antibody or antigen binding fragment thereof comprises the amino acid sequence of SEQ ID NO: 6.

In another aspect, the present disclosure provides a composition comprising 1) the antibody or antigen binding fragment thereof disclosed herein, and 2) an agent that (a) induces cell death, or (b) induces an inflammatory response to a tumor vascular endothelial cell in which the expression of PMEPA-1 is upregulated as compared to expression in a non-tumor vascular endothelial cell control.

In some embodiments, the cell death is induced in a tumor vascular endothelial cell in which the expression of PMEPA-1 is upregulated as compared to expression in a non-tumor vascular endothelial cell control, and/or in a tumor cell.

In certain embodiments, the agent that induces cell death is an agent that induces immunogenic cell death. Alternatively, in other embodiments, the agent that induces cell death is an agent that induces non-immunogenic cell death.

In some embodiments, the agent is selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof.

In some embodiments, the agent that induces an inflammatory response is a TLR4 agonist or GP-130 agonist.

In some embodiments, the agent that induces cell death is a chemotherapeutic agent. In some embodiments, the agent that induces cell death is an engineered CAR-immune cell, optionally the CAR-immune cell is a CAR-T cell, CAR-macrophages, CAR-monocyte, CAR-granulocyte, CAR-NK cell, or a CAR-NKT cell, or a tumor infiltrating lymphocyte (TIL), or a cell expressing an antigen recognizing a tumor antigen or a cell expressing a receptor recognizing an antibody bound to the surface of a tumor cell.

In some embodiments, the engineered CAR-T cell comprises a nucleic acid molecule comprising a nucleotide sequence having at least 90%, 91%, 92%, 93%, 94%, 95%, 96%, 97%, 98%, 99% sequence identity to SEQ ID NOs: 42-47.

In some embodiments, the agent that induces cell death or the agent that induces an inflammatory response is coupled to or is co-administered with the antibody or antigen binding fragment thereof. For example, the agent may be capable of inducing immunogenic cell (e.g., cancer cell) death whereby a subsequent immune response is elicited upon the cell death. Alternatively, the agent may be capable of inducing a non-immunogenic cell (e.g., cancer cell) death, whereby a subsequent immune response is not elicited upon cell death.

In another aspect, the present disclosure provides a pharmaceutical composition comprising 1) the antibody or antigen binding fragment thereof disclosed herein, or the composition disclosed herein, and 2) a pharmaceutically acceptable carrier.

In some embodiments, the pharmaceutical composition comprises a lipid formulation. In some embodiments, the lipid formulation comprises a lipid nanoparticle.

In another aspect, the present disclosure provides a method of treating cancer in a subject in need thereof, wherein the cancer is characterized by a tumor vascular endothelial cell in which the expression PMEPA-1 is upregulated, comprising administering to the subject a composition comprising an antibody or antigen binding fragment thereof which binds to PMEPA-1 on the tumor vascular endothelial cell and an agent that induces cell death or an agent that induces an inflammatory response, optionally wherein the composition is a composition disclosed herein, or the pharmaceutical composition disclosed herein.

In another aspect, the present disclosure provides a method of treating cancer in a subject in need thereof, comprising administering to the subject the composition disclosed herein, or the pharmaceutical composition disclosed herein.

In some embodiments, the expression of the PMEPA-1 is upregulated as compared to a control level.

In some embodiments, the control level is the level of expression of PMEPA-1 in a non-tumor vascular endothelial control cell.

In some embodiments, the method further comprising identifying in the subject the presence of the tumor vascular endothelial cell in which the expression of PMEPA-1 is upregulated as compared to a non-tumor vascular endothelial control cell.

In some embodiments, the method elicits or enhances an immune response to the cancer. In some embodiments, the method increases the level or activity of intra-tumoral T cells.

In some embodiments, the level or activity of intra-tumoral cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

In another aspect, the present disclosure provides a method of treating cancer in a subject in need thereof, comprising administering to a subject having cancer an immune effector cell expressing a chimeric antigen receptor (CAR), wherein the CAR comprises the antibody or antigen binding fragment thereof disclosed herein, wherein the antibody or antigen binding fragment thereof binds to PMEPA-1 on a tumor vascular endothelial cell in which expression of PMEPA-1 is upregulated. In any of these methods, the immune effector cell is a T cell, monocyte, granulocyte, macrophage, natural killer (NK) cell, or natural killer T (NKT).

In another aspect, the present disclosure provides a method of treating cancer in a subject in need thereof, comprising administering to a subject having cancer an immune effector cell expressing a chimeric antigen receptor (CAR), wherein the antibody or antigen binding fragment thereof is expressed on the cell surface of the immune effector cell, wherein the antibody or antigen binding fragment thereof binds to PMEPA-1 on a tumor vascular endothelial cell in which expression of PMEPA-1 is upregulated. In any of these methods, the immune effector cell is a T cell, monocyte, granulocyte, macrophage, natural killer (NK) cell, or natural killer T (NKT).

In some embodiments, the method further comprising identifying in the subject the presence of the tumor vascular endothelial cell in which the expression of PMEPA-1 is upregulated as compared to expression in a non-tumor vascular endothelial control cell.

In some embodiments, the method elicits or enhances an immune response to the cancer. In some embodiments, the method increases the level or activity of intra-tumoral T cells.

In some embodiments, the level or activity of intra-tumoral cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

In another aspect, the present disclosure provides a method of diagnosing or prognosing cancer in a subject, comprising determining the expression of PMEPA-1 on a tumor vascular endothelial cell, wherein upregulation of expression of PMEPA-1 on the tumor vascular endothelial cell as compared to a control level is indicative of the presence or progression of the cancer.

In some embodiments, the control level is the level in a non-tumor endothelial control cell. In some embodiments, the method further comprises a step of administering a cancer treatment.

In another aspect, the present disclosure provides a method of determining the efficacy of treatment of cancer in a subject, comprising i) determining the expression of PMEPA-1 on a tumor vascular endothelial cell after administering a cancer treatment, wherein increased expression of PMEPA-1 as compared to a control level is indicative of the presence or progression of the cancer; ii) determining the expression of PMEPA-1 after administration of the cancer treatment, wherein decreased expression of PMEPA-1 as compared to a control level is indicative of effective cancer treatment.

In some embodiments, the control level is the expression of PMEPA-1 on the tumor vascular endothelial cell prior to administering the cancer treatment.

In some embodiments, the method further comprises the step of administering the cancer treatment.

In some embodiments, the cancer treatment is the composition disclosed herein, or the pharmaceutical composition disclosed herein.

In another aspect, the present disclosure provides a composition comprising the antibody or antigen binding fragment thereof disclosed herein associated with a detectable marker.

In some embodiments, the detectable marker is selected from the group consisting of fluorescent labels, phosphorescent labels, chemiluminescent labels or bioluminescent labels, radio-isotopes, metals, metals chelates or metallic cations, chromophores and enzymes.

In another aspect, the present disclosure provides a medical imaging method comprising (i) administering the composition disclosed herein, and (ii) detecting the antibody or antigen binding fragment thereof in the body of the patient.

In another aspect, the present disclosure provides a use of the composition disclosed herein for the preparation of a medicament for medical imaging in a patient wherein the antibody or antigen binding fragment thereof is detected in the body of the patient.

In another aspect, the present disclosure provides a composition for medical imaging in a patient, wherein the composition comprises the composition disclosed herein and the antibody or antigen binding fragment thereof is detected in the body of the patient.

In some embodiments, the tumor vascular endothelial cell is a venular cell.

In some embodiments, PMEPA-1 is not expressed in non-tumor vascular endothelial cells, wherein PMEPA-1 is expressed at higher levels in tumor vascular endothelial cells as compared to expression in non-tumor vascular endothelial cells, or the expression of PMEPA-1 in tumor vascular endothelial cells is a variant of PMEPA-1 expressed in non-tumor vascular endothelial cells.

In some embodiments, PMEPA-1 is expressed at least 1.5-fold, at least 2-fold, at least 2.5-fold, at least 3-fold, at least 3.5-fold, at least 4-fold, at least 4.5-fold, or at least 5-fold more in tumor vascular endothelial cells as compared to expression in non-tumor vascular endothelial cells.

In another aspect, the present disclosure provides a use of a composition for the preparation of a medicament for treating cancer in a subject in need thereof, wherein the cancer is characterized by a tumor vascular endothelial cell in which the expression of PMEPA-1 is upregulated, and the composition comprises an antibody or antigen binding fragment thereof which binds to PMEPA-1 on the tumor vascular endothelial cell and an agent that induces cell death or an agent that induces an inflammatory response, optionally wherein the composition is a composition disclosed herein, or the pharmaceutical composition disclosed herein.

In another aspect, the present disclosure provides a use of a composition disclosed herein, or the pharmaceutical composition disclosed herein for the preparation of a medicament for treating cancer in a subject in need thereof.

In another aspect, the present disclosure provides a composition for treating cancer in a subject in need thereof, wherein the cancer is characterized by a tumor vascular endothelial cell in which the expression PMEPA-1 is upregulated, and the composition comprises an antibody or antigen binding fragment thereof which binds to PMEPA-1 on the tumor vascular endothelial cell and an agent that induces cell death or an agent that induces an inflammatory response, optionally wherein the composition is a composition disclosed herein, or the pharmaceutical composition disclosed herein.

In another aspect, the present disclosure provides a composition for treating cancer in a subject in need thereof, wherein the composition comprises a composition disclosed herein, or the pharmaceutical composition disclosed herein.

In some embodiments, the expression of the PMEPA-1 is upregulated as compared to a control level. In some embodiments, the control level is the level of expression of PMEPA-1 in a non-tumor vascular endothelial control cell.

In some embodiments, administration of the composition or the medicament elicits or enhances an immune response to the cancer.

In some embodiments, administration of the composition or the medicament increases the level or activity of intra-tumoral T cells.

In some embodiments, the level or activity of intra-tumoral cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

In another aspect, the present disclosure provides a use of a immune effector cell expressing a chimeric antigen receptor (CAR) for the preparation of a medicament for treating cancer in a subject in need thereof, wherein the CAR comprises the antibody or antigen binding fragment thereof disclosed herein, and wherein the antibody or antigen binding fragment thereof binds to PMEPA-1 on a tumor vascular endothelial cell in which expression of PMEPA-1 is upregulated.

In another aspect, the present disclosure provides a composition for treating cancer in a subject in need thereof, wherein the composition comprises a immune effector cell expressing a chimeric antigen receptor (CAR), wherein the CAR comprises the antibody or antigen binding fragment thereof disclosed herein, and wherein the antibody or antigen binding fragment thereof binds to PMEPA-1 on a tumor vascular endothelial cell in which expression of PMEPA-1 is upregulated.

In some embodiments, the subject is identified as having a tumor vascular endothelial cell in which the expression of PMEPA-1 is upregulated as compared to expression in a non-tumor vascular endothelial control cell.

In some embodiments, administration of the composition or the medicament elicits or enhances an immune response to the cancer.

In some embodiments, administration of the composition or the medicament increases the level or activity of intra-tumoral T cells.

In some embodiments, the level or activity of intra-tumoral cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

In some embodiments, the cancer is (i) a non-immunogenic cancer; (ii) a hematological cancer; or (iii) a solid tumor. In some embodiments, the cancer is selected from the group consisting of melanoma, pancreatic cancer, and colorectal cancer. In some embodiments, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

### BRIEF DESCRIPTION OF THE DRAWINGS

FIGS. 1A-1C depicts Venn diagrams of up-regulated genes in murine and human tumor microvasculature compared to healthy tissues. Single cell suspensions of murine MC38 colorectal adenocarcinoma (MC38, blue), murine B16F10 melanoma (B16, red), human pancreatic cancer (hPanT, yellow), human melanoma (hMel, green) and peri-tumoral tissue were isolated by Seq Well and processed for scRNA-seq. V-ECs and NV-ECs were identified based on characteristic gene expression patterns and each EC subset in tumors and matched peri-tumoral tissue was compared to identify tumor-specific over-expressed genes. Venn diagrams of the number of upregulated genes as compared to healthy in (FIG. 1A) all blood ECs, (FIG. 1B) NV-ECs and (FIG. 1C) V-ECs.

FIGS. 2A-2E. FIGS. 2A and 2B depicts validation assays of a candidate tumor EC target, PMEPA-1. EC mRNA levels of PMEPA-1 were compared in VEC, NVEC and lymphatic EC (LEC) in (FIG. 2A) healthy mouse skin and subcutaneous MC38 and B16F10 tumors and (FIG. 2B) human non-malignant pancreas and pancreatic cancer. FIG. 2C FACS analysis of PMEPA-1 on ECs in MC38 tumors and healthy skin. FIG. 2D FACS analysis of PMEPA-1 on ECs in MC38 tumors and healthy skin using Iso control antibody. FIG. 2E Percentage of PMEPA-1+ blood EC (BEC).

FIGS. 3A-3D depicts the generation of nanobody against PMEPA-1. FIG. 3A L1.2 cells were transfected with either a linearized or a circular plasmid. FIG. 3B The cells were expanded in the presence of G418 and GFP expression was assessed by FACS. FIG. 3C Cells with the highest MFI were single sorted and expanded. FIG. 3D Clones demonstrating the highest level of PMEPA-1-GFP expression (which is enhanced by treatment with sodium butyrate) is ready for use in selection of sdAb from the yeast display library.

FIG. 4 shows enrichment of receptor positive cells. Receptor-negative and receptor-positive cells were labeled with different fluorescent dyes and mixed in 1:1 ratio. Yeast expressing sdAb library was added to the cultures in proportion Yeast:Targets=25:1. The cultures were incubated on a gentle shaker at 4° C. for 1 h, the cells were then collected and stained for FACS to determine the percentage of HA+ cells in each population.

FIG. 5 shows a schematic strategy to generate sdAb against PMEPA-1 to target CAR-T cells to solid tumors.

FIGS. 6A-6C depict generation of nanobody-IgG fusion constructs. Individual nanobody CDS were cloned into expression vector pFUSE-mIgG1-Fc2 or pFUSE-hIgG1-Fc2 (mouse and human IgG1 CH2-CH3 domains, respectively, downstream of the IL-2 signal sequence to promote efficient secretion (InvivoGen, San Diego, CA)), generating chimeric proteins as pictured on the right (FIG. 6A). Dot blot analysis of select mouse IgG1 fusion clones to verify the integrity of both the VHH and IgG1 Fc domains. IIB2 (positive clone) and IG12 (negative clone) shown, with purified mouse IgG and purified VHH domain (clone ID5) as controls. 0.5 μL or 2.0 μL tissue culture supernatant spotted as indicated, and probed with HRP anti-mouse IgG (left panel) and HRP anti-VHH (right panel) (FIG. 6B). Flow cytometric analysis of select human (upper panel, probed with PE-labeled anti-human IgG) and mouse (lower panel, probed with APC-labeled anti-mouse IgG) on PMEPA1-expressing 293F cultured cells (FIG. 6C).

FIG. 7 depicts a schematic representation of generation of yeast clones expressing a nanobody (Nb) of interest. Parental yeast library was induced for various Nbs expression and “pre-cleaned” in negative selection: clones bound in anti-biotin beads in the absence of the relevant peptide stick in the column were discarded. Eluted clones undergo positive selection, when they were subsequently incubated with the relevant biotinylated peptide and anti-biotin beads. Clones that stuck in the column were further sorted for Nb-expressing yeast bound to the peptide. The cycle is repeated several times.

FIGS. 8A and 8B depict binding efficiency of Yeast Nb libraries to the m-PMEPA-1 peptide with selection progression. FIG. 8A depicts representative dot plots illustrating binding of Nb-expressing yeast (HA+) to biotinylated peptide (SA+). Double-positive cells (depicted in red quadrants) represent a population of interest. FIG. 8B depicts statistical analysis of data shown in FIG. 8A. Nb-expressing yeast bind to the m-PMEPA-1 peptide more sufficient with each selection. Low or no binding of HA− yeast to the peptide and HA+ yeast to SA in the absence of the peptide indicate that binding is Nb- and peptide-specific.

FIGS. 9A and 9B depicts subclones identified with the highest binding to the m-PMEPA-1 peptide. FIG. 9A shows the screening was performed in 96-well plates, and the results for each subclone is shown as a scatter plot showing the frequency of SA+ population within HA+ yeast and the mean fluorescent intensity of the SA expression. FIG. 9B shows the best and the worst binders are selected and coded according to their position on the plate based on the parameters in FIG. 9A.

FIGS. 10A and 10B depicts dual specificity of subclones for human and mouse PMEPA-1. FIG. 10A shows after screening 384 subclones, several positive and negative candidates were selected and tested again for binding to both m-PMEPA-1 and h-PMEPA-1 peptides (primary amino acid sequences in FIG. 10B) as well as to the corresponding control peptides. FIG. 10B shows the primary amino acid sequence of m-PMEPA-1 and h-PMEPA-1.

FIGS. 11A-11C shows dot blot analysis of Nb:PMEPA-1 binding. Individual mouse and human PMEPA-1-derived peptides, alongside scrambled controls, were blotted onto PVDF membranes (layout in FIG. 11A) and probed with lysate from bacterial cultures expressing either PMEPA-1-specific clone II-B2 (FIG. 11B) or nonspecific clone I-G11 (FIG. 11C).

FIG. 12 depicts fluorescent immunostaining of MC38 tumor vasculature. Frozen sections from subcutaneously implanted MC38 tumor were stained with anti-CD38 and PMEPA-1-specific clone II-B2.

FIG. 13 depicts fluorescent immunostaining of B16-F10 tumor vasculature. Frozen sections from subcutaneously implanted B16-F10 tumor were stained with anti-CD38, DAPI, and PMEPA-1-specific clone II-B2. Bottom panel is a high magnification showing the immunostaining.

FIG. 14 depicts fluorescent immunostaining of B16 lung metastatic tumor vasculature. Frozen sections from B16 lung metastatic tumor were stained with anti-CD31 and PMEPA-1-specific clone II-B2.

FIGS. 15A-15B depict expression of PMEPA 1 on vasculature in lung metastases. FIG. 15A shows expression of PMEPA 1 (depicted by ID5 Nb subclone) on a CD31+ endothelial cells in a vessel adjacent to a lung metastases (M). FIG. 15B demonstrates no expression of PMEPA-1 in normal metastasis-free lung.

FIG. 16 depicts fluorescent immunostaining of human pancreatic tumor vasculature. Frozen sections from human pancreatic cancer and surrounding tissue were stained with anti-CD31 and PMEPA-1-specific clone II-B2.

FIG. 17 depicts information of co-expression of CD31+ vasculature endothelium and additional PMEPA 1-specific nanobody (Nb) clones (ID8 and IIC10) in human pancreatic cancer.

FIGS. 18A-18C depict expression of PMEPA 1 on human papillary thyroid carcinoma. FIG. 18A demonstrates no staining of CD31+ endothelial cells by nanobody (Nb) non-specific clone IG12. FIGS. 18B and 18C depict expression of PMEPA 1 by specific nanobody (Nb) cloned ID5 (FIG. 18B) and IIB2 (FIG. 18C).

FIGS. 19A-19E depict binding of specific and non-specific nanobody (Nb) clones to various human and mouse cell lines by FACS. BW—mouse thymoma cell line, no mRNA PMEPA 1 expression in RT-PCR. The rest of the cell line show PMEPA 1 expression on mRNA level. Human cell lines—Jurkat (T cell leukemia), 293T (human embryonic kidney), PC3 (prostate cancer). Mouse cell line—MEF (mouse embryonic fibroblasts). IG11 or IG12 (FIG. 19A); ID5 (FIG. 19B); ID8 (FIG. 19C); IIB2 (FIG. 19D); and IIC10 (FIG. 19E).

FIG. 20 depicts fluorescent immunostaining of MB459 tumor bearing mice after IV administration of PMEPA-1-specific clone II-B2. Frozen sections from the MB459 tumor harvested 5 minutes after administration were stained with anti-CD31, anti-CD44, DAPI and PMEPA-1-specific clone II-B2.

FIG. 21 depicts targeting of PMEPA-1-specific clone II-B2-coated fluorescent microspheres (1 M) to the MB4459 tumor microvessels after IV administration. Frozen sections from the MB459 tumor harvested 5 minutes after administration were stained with PMEPA-1-specific clone II-B2.

FIG. 22 depicts targeting of PMEPA-1-specific clone II-B2-coated fluorescent microspheres (1 μM) to the extravascular space after IV administration. Frozen sections from the MC tumor harvested 5 minutes after administration of a mixture (1:1) of fluorescent microspheres conjugated to a control nanobody or PMEPA-1-specific clone II-B2 and frozen sections were subjected to fluorescent immunostaining.

### DETAILED DESCRIPTION

The present disclosure provides compositions and methods for treating solid tumors by targeting PMEPA-1, a differentially expressed transmembrane molecule that is upregulated in tumor vascular endothelial cells, such as non-venular and venular endothelial cells in and surrounding a tumor, lining the tumor microvasculature but that is not upregulated in vasculature from healthy tissues, e.g., non-tumor vascular endothelial cells. Characterization of tumor microvasculature, human and murine immune cell infiltrates of immunogenic tumors (T-cell rich and onco-immunotherapy responders), and non-immunogenic tumors (T-cell poor and onco-immunotherapy non-responders) demonstrated the importance of the vasculature in recruiting intra-tumoral T cells. By doing so, PMEPA-1 was identified as a gene that was overexpressed in tumor microvasculature of solid tumors, globally in all tumor vascular endothelial cells within the tumors or selectively in venules or non-venules (capillaries and arterioles).

Thus, the present disclosure provides for methods and compositions i) for targeting overexpressed PMEPA-1, which is a venular surface molecule identified from both immunogenic tumors and non-immunogenic tumors, to target the venules with gene and/or drug delivery for tumor vascular endothelial cell reprogramming so as to increase intra-tumoral T cells, and/or ii) for targeting overexpressed PMEPA-1 to specifically target oncotherapeutic agents (e.g., chemotherapeutic agent or CAR T cells) to venules or non-venules selectively in human solid tumors with minimum off target effects. In particular, the present disclosure also provides targeting molecules, e.g., antibody or antigen binding fragment thereof, e.g., nanobody, to PMEPA-1.

### Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “encode” or “encoding” refers to a property of sequences of nucleic acids, such as a vector, a plasmid, a gene, cDNA, mRNA, to serve as templates for synthesis of other molecules such as proteins.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena. It should be noted that whenever a value or range of values of a parameter are recited, it is intended that values and ranges intermediate to the recited values are also intended to be part of this invention.

As used herein, the term “tumor vascular endothelial cell” refers to endothelial cells that are associated with a tumor. Vascular endothelial cells form the lining of the inner surface of all blood vessels, and constitute a non-thrombogenic interface between blood and tissue. In addition, vascular endothelial cells are an important component for the development of new capillaries and blood vessels. Tumor vascular endothelial cells proliferate during the angiogenesis, or neovascularization, associated with tumor growth and metastasis. Tumor vascular endothelial cells are associated with new capillaries and blood vessels associated with tumors. (See Dudley A C. Tumor endothelial cells. Cold Spring Harb Perspect Med. 2012; 2(3):a006536.). The tumor vascular endothelial cells include both venular and non-venular endothelial cells found in or surrounding tumor.

As used herein, the term “non-tumor vascular endothelial cell” refers to endothelial cells that are not associated with a tumor and, for example, are found in normal “healthy” tissues.

The vascular endothelium is a dynamic cellular “organ” that controls passage of nutrients into tissues, maintains the flow of blood, and regulates the trafficking of leukocytes (e.g., T cell). In normal tissues, the endothelial cells form a continuous and uniform monolayer, while tumor endothelial cells are irregular in shape and size and have cytoplasmic projection extending into the vessel lumen. Tumor vascular endothelial cells can block T cells from entry into the tumor through the deregulation of adhesion molecules in the vessels. (See Lanitis E, Irving M, Coukos G. Targeting the tumor vasculature to enhance T cell activity. Curr Opin Immunol. 2015; 33:55-63.).

As used herein, a “targeting molecule” refers to any molecule that binds to a component associated with an organ, tissue, cell, extracellular matrix, and/or subcellular locale. In some embodiments, such a component is referred to as a “target” or a “marker”. In some embodiments, the target or marker is PMEPA-1.

In some embodiments, a targeting molecule, e.g., antibody or antigen binding fragment thereof, in accordance with the present invention may be a polypeptide. In certain embodiments, polypeptides range from about 75 to about 300, from about 100 to about 250, from about 100 to about 200, from about 75 to about 250, or from about 75 to about 200 amino acids in size.

The terms “polypeptide” and “peptide” are used interchangeably herein, with “peptide” typically referring to a polypeptide having a length of less than about 100 amino acids. Polypeptides may contain L-amino acids, D-amino acids; or both and may contain any of a variety of amino acid modifications or analogs known in the art. Useful modifications include, e.g., terminal acetylation, amidation, lipidation, phosphorylation, glycosylation, acylation, farnesylation, sulfation, etc.

As used herein, the terms “fragment,” “derivative,” and “analog” refer to a polypeptide that substantially retains the same biological function or activity of a protein, e.g., nanobody, of the invention. Polypeptide fragments, derivatives or analogs of the invention may be (i) polypeptides having one or more conservative or non-conservative amino acid residues (preferably non-conservative amino acid residues) substituted. Such substituted amino acid residues may or may not be encoded by the genetic code; or (ii) a polypeptide having a substituent group in one or more amino acid residues; or (iii) a polypeptide formed by fusing a mature polypeptide and another compound (such as a compound that increases the half-life of the polypeptide, for example, polyethylene glycol); or (iv) a polypeptide formed by fusing an additional amino acid sequence to the polypeptide sequence (e.g., a leader or secretory sequence or a sequence used to purify this polypeptide or a proprotein sequence, or a fusion protein formed with a His tag). According to the teachings herein, these fragments, derivatives, and analogs are within the scope of one of ordinary skill in the art.

Exemplary proteins that may be used as targeting molecules in accordance with the present invention include, but are not limited to, antibodies, or antigen-binding fragments thereof, receptors, cytokines, peptide hormones, glycoproteins, glycopeptides, proteoglycans, proteins derived from combinatorial libraries (e.g., Avimers™, Affibodies®, etc.), and characteristic portions thereof. Synthetic binding proteins such as Nanobodies™ AdNectins™, etc., can be used. In some embodiments, protein targeting molecules can be a nanobody.

One of ordinary skill in the art will appreciate that any protein and/or peptide that specifically binds to a desired target as described herein, can be used in accordance with the present invention.

### Antibody or Antigen Binding Fragments to PMEPA-1

The present invention provides for an antibody and/or antigen binding fragment thereof, for example, a nanobody, to PMEPA-1. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE. Antibodies or immunoglobulins are heterotetrameric glycosaminoglycan proteins of about 150,000 Dalton with the same structural features, consisting of two identical light (L) chains and two identical heavy (H) chains. Each light chain is linked to the heavy chain through a covalent disulfide bond, and the number of disulfide bonds between the heavy chains of different immunoglobulin isoforms is different. Each heavy and light chain also has intra-chain disulfide bonds which are regular spaced. Each heavy chain has a variable region (VH) at one end followed by a plurality of constant regions. Each light chain has a variable region (VL) at one end and a constant region at the other end; the constant region of the light chain is opposite to the first constant region of the heavy chain, and the variable region of the light chain is opposite to the variable region of the heavy chain. Special amino acid residues form an interface between the variable regions of the light and heavy chains.

As used herein, an “antibody fragment” or “antigen binding fragment” (i.e. characteristic portion of an antibody) refers to any derivative of an antibody which is less than full-length. In some embodiments, an antibody fragment retains at least a significant portion of the full-length antibody's specific binding ability. Examples of such antibody fragments include, but are not limited to, Fab, Fab′, F(ab′)2, scFv, Fv, dsFv diabody, VHH, and Fd fragments. Antibody fragments also include, but are not limited, to Fc fragments.

As used herein, the terms “single domain antibody (VHH)” and “nanobodies” have the same meaning referring to a variable region of a heavy chain of an antibody, and construct a single domain antibody (VHH) consisting of only one heavy chain variable region. It is the smallest antigen-binding fragment with complete function. Generally, the antibodies with a natural deficiency of the light chain and the heavy chain constant region 1 (CH1) are first obtained, the variable regions of the heavy chain of the antibody are therefore cloned to construct a single domain antibody (VHH) consisting of only one heavy chain variable region.

As used herein, the term “variable” refers that certain portions of the variable region in the nanobodies vary in sequences, which forms the binding and specificity of various specific antibodies to their particular antigen. However, variability is not uniformly distributed throughout the nanobody variable region. It is concentrated in three segments called complementarity-determining regions (CDRs) or hypervariable regions in the variable regions of the light and heavy chain. The more conserved part of the variable region is called the framework region (FR). The variable regions of the natural heavy and light chains each contain four FR regions, which are substantially in a β-folded configuration, joined by three CDRs which form a linking loop, and in some cases can form a partially β-folded structure. The CDRs in each chain are closely adjacent to the others by the FR regions and form an antigen-binding site of the nanobody with the CDRs of the other chain (see Kabat et al., NIH Publ. No. 91-3242, Volume I, pages 647-669. (1991)). The constant regions are not directly involved in the binding of the nanobody to the antigen, but they exhibit different effects or functions, for example, involve in antibody-dependent cytotoxicity of the antibodies.

As used herein, the term “heavy chain variable region” and “VI” can be used interchangeably. As used herein, the terms “variable region” and “complementary determining region (CDR)” can be used interchangeably.

An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

In some embodiments, antibodies may include chimeric (e.g. “humanized”) and single chain (recombinant) antibodies. In some embodiments, antibodies may have reduced effector functions and/or bispecific molecules. In some embodiments, antibodies may include fragments produced by a Fab expression library.

In a particular embodiment, the antibody or antigen binding fragment is a nanobody. Nanobodies are recombinant antibody fragments consisting of one variable heavy chain. In some embodiments, the variable heavy chain of a nanobody comprises a CDR1, CDR2, and CDR3. The CDR1 and CDR2 segments can be short in comparison to the CDR3 segment, which is longer than the typical CDR3 in a conventional antibody or scFv molecule. In some embodiments, the nanobodies can comprise multiple (two or more) VH segments, such as a dimer. Peptide linker can be between VH segments. Each VH segment in a multimer nanobody can be the same VH sequence binding to the same antigen, or different VH sequence binding to different antigens, or different VH sequences binding the same antigen at non-overlapping epitopes. In some embodiments, the nanobodies can comprise multiple segments of VH segments as described above and scFv molecules. In some embodiments, the nanobodies can be covalently linked to a drug (e.g., chemotherapeutic drug), imaging probe, or displayed on the surface of nanoparticles, viruses, or CAR T cells.

The present disclosure also provides other polypeptides, such as a fusion protein comprising nanobodies or fragments thereof. Typically, the fragment has at least about 50 contiguous amino acids of the nanobody as disclosed herein, preferably at least about 50 contiguous amino acids, more preferably at least about 80 contiguous amino acids, and most preferably at least about 100 contiguous amino acids.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

An F(ab′)2 fragment is an antibody fragment essentially equivalent to that obtained from immunoglobulins by digestion with an enzyme pepsin at pH 4.0-4.5. The fragment may be recombinantly produced.

A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

A Fab fragment is an antibody fragment essentially equivalent to that obtained by digestion of immunoglobulins with an enzyme (e.g., papain). The Fab fragment may be recombinantly produced. The heavy chain segment of the Fab fragment is the Fd piece.

In some embodiments, the antibodies or antigen-binding fragments thereof comprise a variant of the disclosed antibodies or antigen-binding fragments thereof herein, for example, antibodies or antigen-binding fragments thereof in which there are up to 10, preferably up to 8, more preferably up to 5, and most preferably up to 3 amino acids substituted by amino acids having analogical or similar properties, compared to the amino acid sequences set forth herein. These conservative variant antibodies or antigen-binding fragments thereof are preferably produced according to the amino acid substitutions in Table 1.

TABLE 1

| Original Residue | Possible substitution |
|---|---|
| Ala (A) | Val; Leu; Ile |
| Arg (R) | Lys; Gln; Asn |
| Asn (N) | Gin; His; Lys; Arg |
| Asp (D) | Glu |
| Cys (C) | Ser |
| Gln (Q) | Asn |
| Glu (E) | Asp |
| Gly (G) | Pro; Ala |
| His (H) | Asn; Gln; Lys; Arg |
| Ile (I) | Leu; Val; Met; Ala; Phe |
| Len (L) | Ile; Val; Met; Ala; Phe |
| Lys (K) | Arg; Gln; Asn |
| Met (M) | Leu; Phe; Ile |
| Phe (F) | Leu; Val; Ile; Ala; Tyr |
| Pro (P) | Ala |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

In accordance with the present invention, the antibody, or antigen binding fragment thereof such as a nanobody, recognizes PMEPA-1. PMEPA-1, which can be recognized by the antibody, or antigen binding fragment thereof, e.g., nanobody, is a cell type specific marker that may be expressed at levels at least 1.5-fold, at least 2-fold, at least 2.5-fold, at least 3-fold, at least 3.5-fold, at least 4-fold, at least 4.5-fold, or at least 5-fold greater in tumor vascular endothelial cells than in a reference population of cells (e.g., non-tumor vascular endothelial cells) which may consist, for example, of a mixture containing an approximately equal amount of cells (e.g., approximately equal numbers of cells, approximately equal volume of cells, approximately equal mass of cells, etc.). In some embodiments, the cell type specific marker PMEPA-1 is present at levels at least 1.5 fold, at least 2 fold, at least 3 fold, at least 4 fold, at least 5 fold, at least 6 fold, at least 7 fold, at least 8 fold, at least 9 fold, at least 10 fold, at least 50 fold, at least 100 fold, at least 500 fold, at least 1000 fold, at least 5000 fold, or at least 10,000 fold greater than its average expression in a reference population. Detection or measurement of the cell type specific marker PMEPA-1 may make it possible to distinguish the cell type or types of interest from cells of many, most, or all other types.

In a particular embodiment, the claims are directed to a nanobody targeting PMEPA-1. In various embodiments, the nanobody comprises
(i) a CDR1 sequence comprising the amino acid sequence of SEQ ID NO: 14, a CDR2 sequence comprising the amino acid sequence of SEQ ID NO: 15, and a CDR3 sequence comprising the amino acid sequence of SEQ ID NO: 16;
(ii) a CDR1 sequence comprising the amino acid sequence of SEQ ID NO: 17, a CDR2 sequence comprising the amino acid sequence of SEQ ID NO: 18, and a CDR3 sequence comprising the amino acid sequence of SEQ ID NO: 19;
(iii) a CDR1 sequence comprising the amino acid sequence of SEQ ID NO: 20, a CDR2 sequence comprising the amino acid sequence of SEQ ID NO: 21, and a CDR3 sequence comprising the amino acid sequence of SEQ ID NO: 22;
(iv) a CDR1 sequence comprising the amino acid sequence of SEQ ID NO: 23, a CDR2 sequence comprising the amino acid sequence of SEQ ID NO: 24, and a CDR3 sequence comprising the amino acid sequence of SEQ ID NO: 25;
(v) a CDR1 sequence comprising the amino acid sequence of SEQ ID NO: 26, a CDR2 sequence comprising the amino acid sequence of SEQ ID NO: 27, and a CDR3 sequence comprising the amino acid sequence of SEQ ID NO: 28; or
(vi) a CDR1 sequence comprising the amino acid sequence of SEQ ID NO: 29, a CDR2 sequence comprising the amino acid sequence of SEQ ID NO: 30, and a CDR3 sequence comprising the amino acid sequence of SEQ ID NO: 31.

In some embodiments, the nanobody comprises an amino acid sequence that has at least 90%, 91%, 92%, 93%, 94%, 95%, 96%, 97%, 98%, or 99% identity to, comprises, or consists of an amino acid sequence selected from SEQ ID NOs: 1-7.

In one embodiment, the nanobody comprises an amino acid sequence of SEQ ID NO: 1. In one embodiment, the nanobody comprises an amino acid sequence of SEQ ID NO: 2. In one embodiment, the nanobody comprises an amino acid sequence of SEQ ID NO: 3. In one embodiment, the nanobody comprises an amino acid sequence of SEQ ID NO: 4. In one embodiment, the nanobody comprises an amino acid sequence of SEQ ID NO: 5. In one embodiment, the nanobody comprises an amino acid sequence of SEQ ID NO: 6. In one embodiment, the nanobody comprises an amino acid sequence of SEQ ID NO: 7.

In one embodiment, the nanobody is encoded by a nucleic acid comprising the nucleotide sequence of SEQ ID NO: 42. In one embodiment, the nanobody is encoded by a nucleic acid comprising the nucleotide sequence of SEQ ID NO: 43. In one embodiment, the nanobody is encoded by a nucleic acid comprising the nucleotide sequence of SEQ ID NO: 44. In one embodiment, the nanobody is encoded by a nucleic acid comprising the nucleotide sequence of SEQ ID NO: 45. In one embodiment, the nanobody is encoded by a nucleic acid comprising the nucleotide sequence of SEQ ID NO: 46. In one embodiment, the nanobody is encoded by a nucleic acid comprising the nucleotide sequence of SEQ ID NO: 47.

In some embodiments, the nanobody includes one or more variations. These variations include, but are not limited to, deletion, insertions and/or substitutions of 1, 2, 3, 4, 5, 6, 7, 8, 9 or 10 (usually 1-50, preferably 1-30, more preferably 1-20, optimally 1-10) amino acids, and addition of 1, 2, 3, 4, 5, 6, 7, 8, 9, or 10 (generally less than 20, preferably less than 10, and more preferably less than 5) amino acids at C-terminus and/or N-terminus. For example, in the art, the substitution of amino acids with analogical or similar properties usually does not alter the function of the protein. By way of further example, addition of one or several amino acids at the C-terminus and/or N-terminus usually does not change the function of the protein. The term also includes active fragments and active derivatives of the disclosed nanobodies.

In some embodiments, the antibody or antigen binding fragment thereof, e.g., nanobody, to PMEPA-1 are coupled (e.g., covalently associated) with an agent that is capable of inducing cell death to a tumor vascular endothelial cell in which the expression of PMEPA-1 (and, optionally, an additional transmembrane molecule from Tables 2-4) is upregulated as compared to a non-tumor vascular endothelial cell. In some embodiments, covalent association is mediated by a linker. In some embodiments, the antibody or an antigen binding fragment thereof, e.g., nanobody, to PMEPA-1 is not covalently associated with an agent that is capable of inducing cell death to a tumor vascular endothelial cell in which the expression of PMEPA-1 (and, optionally, an additional transmembrane molecule from Tables 2-4) is upregulated as compared to a non-tumor vascular endothelial control cell.

The antibody or antigen binding fragment thereof, e.g., nanobody, to PMEPA-1 may be associated with the surface of, encapsulated within, surrounded by, and/or distributed throughout the lipid formulation or polymeric matrix of an lipid nanoparticle, nanosphere, nanocarrier, microsphere, or microparticle. For example, in some embodiments, an antibody or antigen binding fragment thereof, e.g., nanobody, to PMEPA-1, can be encapsulated within, surrounded by, and/or dispersed throughout the liposomal membrane and/or polymeric matrix of a lipid nanoparticle, nanosphere, nanocarrier, microsphere, or microparticle. Alternatively or additionally, an antibody or antigen binding fragment thereof, e.g., nanobody, to PMEPA-1 can be associated with a lipid nanoparticle, nanosphere, nanocarrier, microsphere, or microparticle by charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof.

The nanoparticles, nanospheres, nanocarriers, microparticles or microspheres may comprise one or more of polysaccharides, proteins, lipids, chitosan, alginate, pectin, xanthan gum, and cellulose. The nanoparticles, nanospheres or nanocarriers may be liposomes, polymeric micelles, dendrimers. Exemplary dendrimers include those comprising poly-L-lysine, olyamidoamine (PAMAM), polypropylene imine (PPI), liquid crystalline, core-shell, chiral, peptide, glycodendrimers and PAMAMOS dendrimers. Alternatively, the nanoparticles, nanospheres, nanocarrier, microparticles or microspheres may comprise an inorganic compound such as silver, gold, iron oxide, silica, zinc oxide, titanium oxide, platinum, selenium, gadolinium, palladium, or cerium dioxide.

In some embodiments, the antibody, or antigen binding fragment thereof, e.g., nanobody, to PMEPA-1 is covalently linked to a lipid nanoparticle, nanosphere, nanocarrier, microsphere or microparticle. For example, the antibody, or antigen binding fragment thereof, e.g., nanobody, to PMEPA-1 is linked to a nanoparticle, nanosphere, nanocarrier, microsphere or microparticle by a peptide linker. Exemplary peptide linkers include the dipeptide Val Cit (VC), the tripeptide AAN, or longer peptide such as (GGGGS)(n=1, 2, 3, or 4) (SEQ ID NO: 51), (Gly)8 (SEQ ID NO: 52), (Gly)6 (SEQ ID NO: 53), (EAAAK)3 (SEQ ID NO: 54), (EAAAK)(n=1-3) (SEQ ID NO: 55), A(EAAAK)4ALEA(EAAAK)4A (SEQ ID NO: 56), PAPAP (SEQ ID NO: 57), AEAAAKEAAAKA (SEQ ID NO: 58), (Ala-Pro)n (10-34 aa) (SEQ ID NO: 59). Other types of linkers include GPI-anchors and cross-linked polymers.

Alternatively, the antibody, or antigen binding fragment thereof, e.g., nanobody, to PMEPA-1 is linked to a nanoparticle, nanosphere, nanocarrier, microsphere or microparticle by a cleavable linker such as an acid-labile linker, a protease cleavable linker, an enzyme cleavable linker, or a reducible disulfide linkage. Exemplary cleavable linkers include those comprising an ester bond such as a glutaryl linker, those comprising an amide bond and those comprising a carbamate bond. An exemplary acid-labile linker are hydrozone linkers.

In addition, the antibody, or antigen binding fragment thereof, e.g., nanobody, to PMEPA-1 is linked to a nanoparticle, nanosphere, nanocarrier, microsphere or microparticle by an uncleavable such as an amide bond and a succinimidyl thioester linker or an amide bond and triazole linker or an oxime linker or a triazole linker.

In some embodiments, the antibody, or antigen binding fragment thereof, e.g., nanobody, to PMEPA-1 is covalently linked to one or more detectable markers (e.g., imaging probe or detectable labels) or other signal-generating groups or moieties, depending on the intended use of the labeled antibody, or antigen binding fragment thereof, e.g., nanobody. Suitable markers and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy.

In some embodiments, the invention provides for a targeting molecule that binds PMEPA-1 in addition to at least one other target molecule, for example, another target molecule listed in Tables 2-4. Alternatively or in combination, an antibody, or antigen binding fragment thereof, e.g., nanobody, targeting PMEPA-1 is co-administered with a targeting molecule, e.g., antibody or antigen binding fragment thereof, e.g., nanobody, that binds a target molecule listed in Tables 2-4. Tables 2-4 provide lists of clinically relevant plasma membrane molecules that are overrepresented in all endothelial cells (Table 2) and specific segment of the vasculature such as tumor venular endothelial cells (Table 3) and tumor non-venular endothelial cells (Table 4) from murine and human tumors compared to their respective non-malignant tissues, as described in U.S. Provisional Application No. 63/282,565 (filed Nov. 23, 2021 and entitled Compositions and Methods for Treating Cancer by Targeting Endothelial Cells having Upregulated Expression of Transmembrane Molecules, the entire contents of which are incorporated by reference herein).

TABLE 2
Plasma membrane molecules over-represented in EC from tumor vs
healthy shared by human melanoma and pancreatic tumor (6 genes):
ENTPD1, MARCKS, SELP, APLNR, ROBO1, PLEKHO1
Plasma membrane molecules over-represented in EC from tumor vs
healthy unique to human melanoma (57 genes):
BTN3A2, SLCO2A1, SLC35G2, TNFSF10, PLIN2, ENG, PLVAP, PODXL, PPAP2A,
RAMP3, KDR, HLA-C, SLC6A6, INSR, TGFBR2, MLEC, HLA-DRA, VASP,
C1QTNF5, EHD4, ITGA2, HLA-DRB1, IFITM3, EFNA1, CALCRL, F2R, RELL1,
VAMP5, CD40, SLC30A1, NRP1, HLA-DOA, ESAM, THY1, BMPR2, ACVRLI,
TM2B, MOB1A, SFRP1, SLC38A2, HEG1, CD99, PPAP2B, SPRY4, ATP8B1, FZD6,
ANXA5, CNIH1, DLL4, CSF2RB, CD164, TMEM165, PLXND1, NT5E, RAB13,
CD200, TMED10
Plasma membrane molecules over-represented in EC from tumor vs
healthy unique to human pancreatic tumor (23 genes):
CNKSR3, ASPH, STAB1, KCTD12, LEPR, PTP4A3, SVIL, ENPP2, TGFBR3, ITPR2,
DSP, FAP, BACE2, NRP2, CADM3, ACKR1, THSD7A, DST, CD93, SULF2, MCTP1,
ADGRG6, TIE1
Plasma membrane molecules over-represented in EC from tumor vs
healthy shared by human pancreatic tumor, murine melanoma and/or
murine colorectal cancer (9 genes):
VMP1, LAPTM5, EVL, PCDH17, ARRDC3, PMEPA-1, MYOF, MMP14, PLEKHO1
Plasma membrane molecules over-represented in EC from tumor vs
healthy shared by human melanoma, murine melanoma and/or murine
colorectal cancer (8 genes):
ACTR3, CD74, CLIC1, LAPTM4B, HLA-DQA1, TAPBP, MCAM, PLEKHO1
Plasma membrane molecules over-represented in EC from tumor vs
healthy shared by human melanoma, human pancreatic tumor, murine
melanoma and murine colorectal cancer (1 genes):
PLEKHO1

TABLE 3
Plasma membrane molecules over-represented in V-EC from tumor vs
healthy shared by human melanoma and pancreatic tumor (17 genes):
ENG, KDR, INSR, NRP1, ACVRLI, ROBO1, PKP4, CD200, ITGA2, CSF2RB,
ENTPD1, RELL1, TNFRSFIB, LAPTM4B, MARCKS, DYSF, PLXND1
Plasma membrane molecules over-represented in V-EC from tumor vs
healthy unique to human melanoma (50 genes):
SIGIRR, PTAFR, RAPIA, HLA-DMA, SYPL1, FAT4, HLA-DRB1, CERK, SYT15,
CPD, PTPRN2, HLADOA, THY1, FZD6, CNIH1, HLA-DQB2, IL3RA, BTN3A2,
SLCO2A1, TNFSF10, PLVAP, GPR68, CLSTN3, RAMP3, KL, HLA-C, HLA-DPB1,
GBP5, DIAPH1, HLA-DRA, EHD4, TMEM59, CX3CL1, ATP8A1, TSPAN7, LRRC8A,
CD40, TACR1, IL10RB, SLC30A1, SFRP1, BTN3A3, IL17RA, YIPF3, DLL1, ABI3,
SEMA4C, SLC29A1, RAB13, TMED10
Plasma membrane molecules over-represented in V-EC from tumor vs
healthy unique to human pancreatic tumor (63 genes):
TLR4, LEPR, ITGA5, ESYT1, RAC1, PAM, GNA14, ORAI2, ADGRLA, ASAP1,
CADPS2, TGFBR3, LRRC32, DSP, MET, LRP6, PPFIA1, OSBPL8, KRIT1, ANO2,
PGRMC1, CLDN5, EPS8, ADCY4, TMEM127, GRK5, IL13RA1, PLXDC2, NECTIN2,
CADM3, PON2, ACKR1, F2RL3, ITGA6, MFAP3, TIE1, CNKSR3, TJP1, FZD4,
ENPP2, C1QTNF5, ITPR2, CALCRL, EFNB2, CLECIA, PNN, BACE2, ATP1B3, NRP2,
FLT4, ITGA1, PPP4R3A, GPR146, CTTN, CLTC, ATP2B4, ERLIN1, RIT1, USP9X,
MCTP1, ADGRG6, ADGRF5, NCKAP1
Plasma membrane molecules over-represented in V-EC from tumor vs
healthy shared by human pancreatic tumor, murine melanoma and/or
murine colorectal cancer (22 genes):
PCDH1, THSD7A, STAB1, PTPRG, PHACTR4, MLEC, GPR107, SEMA3F, CD93,
EVL, PCDH17, VMP1, BST2, MMP14, TM9SF2, ENTPD1, RELL1, TNFRSFIB,
LAPTM4B, MARCKS, DYSF, PLXND1
Plasma membrane molecules over-represented in V-EC from tumor vs
healthy shared by human melanoma, murine melanoma and/or murine
colorectal cancer (23 genes):
TGFBR2, ESAM, IFNAR1, CD74, VAMP5, APLNR, HLA-DQA1, TMEM204, PCDH12,
MPZL1, F2R, GLG1, CLIC1, ACTR3, AMOTL1, PLEKHO1, MARCKS, DYSF,
PLXND1, ENTPD1, RELL1, TNFRSFIB, LAPTM4B, ACTR3, AMOTL1, PLEKHO1

TABLE 4
Plasma membrane molecules over-represented in NV-EC from tumor vs
healthy shared by human melanoma and pancreatic tumor (7 genes):
ENTPD1, MARCKS, APLNR, ROBO1, CD93, PCDH17, PLEKHO1
Plasma membrane molecules over-represented in NV-EC from tumor vs
healthy unique to human melanoma (71 genes):
APCDD1, JAG2, STX3, SLC35G2, HECW2, PLIN2, ENG , PLVAP, PODXL, RAMP3,
MPZL2, KDR, HLA-C, SLC6A6, INSR, TGFBR2, PLPP3, MLEC, HLA-DRA, VASP,
JCAD, C1QTNF5, ITGA2, MAGED2, HLA-DRB1, IFITM3, EFNA1, B4GALT1,
CALCRL, F2R, VAMP5, TSPAN12, LGALS9, PLPP1, TMEM30A, SLC30A1, GNB2,
SELP, NRP1, FLT4, ESAM, ABHD12, BMPR2, ACVRL1, ADGRL2, ITM2B, MOB1A,
PKD2, SFRP1, SLC38A2, CDH5, HEG1, CD99, CLIC4, GRK5, SPRY4, CLTC,
ATP8B1, TAPBP, CNIH1, APP, DLL4, FLRT2, DYSF, CD164, TMEM165, PLXND1,
RAB13, GPR4, CD200, BSG
Plasma membrane molecules over-represented in NV-EC from tumor vs
healthy unique to human pancreatic tumor (30 genes):
STAB1, DLG1, KCTD12, LEPR, CACNAIC, PTP4A3, VMP1, TM4SF1, ATP11C,
SVIL, SLC4A7, ENPP2, DSP, PTPRE, NRP2, JAG1, EVL, PCSK5, SLC26A2, ACKR1,
ATP2B1, THSD7A, DST, ABCB1, FBLIM1, MCTP1, SULF2, ARRDC3, TIE1, KCNN3
Plasma membrane molecules over-represented in NV-EC from tumor vs
healthy shared by human pancreatic tumor, murine melanoma and/or
murine colorectal cancer (6 genes):
FCGR2A, LAPTM5, PCDH17, PLEKHO1, PMEPA-1, MMP14
Plasma membrane molecules over-represented in NV-EC from tumor vs
healthy shared by human melanoma, murine melanoma and/or murine
colorectal cancer (13 genes):
SLCO2A1, VCAM1, GJA4, CD74, LAPTM4B, NT5E, TNFAIP1, EDNRB, ANXA5,
PCDH17, PLEKHO1, MCAM, CLIC1
Plasma membrane molecules over-represented in NV-EC from tumor vs
healthy shared by human melanoma, human pancreatic tumor, murine
melanoma and murine colorectal cancer (1 genes):
PLEKHO1

### Expression of Antibody or Antigen Binding Fragment

The present invention also provides a polynucleotide molecule encoding the antibody or antigen binding fragment thereof, e.g., nanobody, to PMEPA-1. In some embodiments, the polynucleotide encodes an antibody or antigen-binding fragment thereof, to PMEPA-1. In a particular embodiment, the polynucleotide encodes a nanobody to PMEPA-1. In some embodiments, the polynucleotide may be in the form of DNA or RNA. DNA forms include cDNA, genomic DNA, or synthetic DNA. DNA can be single-stranded or double-stranded. DNA can be a coding strand or a non-coding strand.

In various embodiments, the present invention provides for a polynucleotide molecule encoding a nanobody comprising
(i) a CDR1 sequence comprising the amino acid sequence of SEQ ID NO: 14, a CDR2 sequence comprising the amino acid sequence of SEQ ID NO: 15, and a CDR3 sequence comprising the amino acid sequence of SEQ ID NO: 16;
(ii) a CDR1 sequence comprising the amino acid sequence of SEQ ID NO: 17, a CDR2 sequence comprising the amino acid sequence of SEQ ID NO: 18, and a CDR3 sequence comprising the amino acid sequence of SEQ ID NO: 19;
(iii) a CDR1 sequence comprising the amino acid sequence of SEQ ID NO: 20, a CDR2 sequence comprising the amino acid sequence of SEQ ID NO: 21, and a CDR3 sequence comprising the amino acid sequence of SEQ ID NO: 22;
(iv) a CDR1 sequence comprising the amino acid sequence of SEQ ID NO: 23, a CDR2 sequence comprising the amino acid sequence of SEQ ID NO: 24, and a CDR3 sequence comprising the amino acid sequence of SEQ ID NO: 25;
(v) a CDR1 sequence comprising the amino acid sequence of SEQ ID NO: 26, a CDR2 sequence comprising the amino acid sequence of SEQ ID NO: 27, and a CDR3 sequence comprising the amino acid sequence of SEQ ID NO: 28; or
(vi) a CDR1 sequence comprising the amino acid sequence of SEQ ID NO: 29, a CDR2 sequence comprising the amino acid sequence of SEQ ID NO: 30, and a CDR3 sequence comprising the amino acid sequence of SEQ ID NO: 31.

In some embodiments, the polynucleotide molecule encodes a nanobody comprising an amino acid sequence that has at least 90%, 91%, 92%, 93%, 94%, 95%, 96%, 97%, 98%, or 99% identity to, comprises, or consists of an amino acid sequence selected from SEQ ID NOs: 1-7.

In various embodiments, the polynucleotide molecule encodes a nanobody comprising an amino acid sequence of SEQ ID NO: 1, SEQ ID NO: 2, SEQ ID NO: 3, SEQ ID NO: 4, SEQ ID NO: 5, SEQ ID NO: 6 or SEQ ID NO: 7.

In various embodiments, the polynucleotide molecule comprises the nucleotide sequence of SEQ ID NO: 42, SEQ ID NO: 43, SEQ ID NO: 44, SEQ ID NO: 45, SEQ ID NO: 46, or SEQ ID NO: 47.

In some embodiments, the polynucleotide molecule is contained in a vector. In some embodiments, the vectors comprise suitable promoters and/or control sequences. These vectors can be used to transform an appropriate host cell so that it can express the protein (e.g., antibody or antigen-binding fragment, thereof, e.g., a nanobody to PMEPA-1).

In some embodiments, the host cell can be a prokaryotic cell (e.g., a bacterial cell); a eukaryotic cell (e.g., a yeast cell, insect cell, or mammalian cell). In some embodiments, the prokaryotic cell or eukaryotic cell comprises the polynucleotide molecule encoding the antibody or antigen-binding fragment thereof, e.g., a nanobody, to PMEPA-1. In some embodiments, the prokaryotic cell or eukaryotic cell expresses the antibody or antigen-binding fragment thereof, e.g., a nanobody, to PMEPA-1 for purification of the antibody or antigen-binding fragment thereof. In some embodiments, the prokaryotic cell or eukaryotic cell expresses the antibody or antigen-binding fragment thereof, e.g., a nanobody, to PMEPA-1 for displaying the antibody or antigen-binding fragment thereof on the surface of the cell.

### Viral Vector

The term “viral vector” refers to a nucleic acid molecule that includes virus-derived nucleic acid elements that facilitate transfer and expression of non-native nucleic acid molecules within a cell. For example, a viral vector, such as an AAV viral vector can be used to express the antibody, or antigen binding fragment thereof, e.g., a nanobody, to PMEPA-1. In a particular embodiment, a viral vector, such as an AAV viral vector can be used to express the antibody, or antigen binding fragment thereof, e.g., a nanobody, to PMEPA-1, in CAR-T cells.

The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

As used herein, the term “adenovirus vector” refers to those constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. As used herein, the term “AAV vector” in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388), which are also encompassed by the term “AAV.” Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In some embodiments, adenovirus type 5 of subgroup C is the preferred starting material in order to obtain a conditional replication-defective adenovirus vector for use in some embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in selected cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retrovirus. Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in some embodiments include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus. “Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In some embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In some embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al, 1999, J. Virol., 73:2886); the posttranscriptional regulatory element present in hepatitis B virus (HPRE) (Smith et al., Nucleic Acids Res. 26(21):4818-4827, 1998); and the like (Liu et al., 1995, Genes Dev., 9: 1766). In some embodiments, vectors include a posttranscriptional regulatory element such as a WPRE or HPRE. In some embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In some embodiments, vectors include a polyadenylation sequence 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40 pA. In some embodiments, a preferred embodiment of an expression construct includes a terminator element. These elements can serve to enhance transcript levels and to minimize read through from the construct into other plasmid sequences.

In some embodiments, a viral vector further includes one or more insulator elements. Insulator elements may contribute to protecting viral vector-expressed sequences, e.g., effector elements or expressible elements, from integration site effects, which may be mediated by as-acting elements present in genomic DNA and lead to deregulated expression of transferred sequences (i.e., position effect; see, e.g., Burgess-Beusse et al, PNAS., USA, 99: 16433, 2002; and Zhan et al., Hum. Genet., 109:471, 2001). In some embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken b-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33: 198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In some embodiments, suitable expression vectors include any plasmid, cosmid or phage construct that is capable of supporting expression of encoded genes in mammalian cell, such as pUC or Bluescript plasmid series.

In some embodiments, vectors (e.g., AAV) include AAV9 (Gombash et al., Front Mol Neurosci. 2014; 7:81), AAVrh.10 (Yang, et al., Mol Ther. 2014; 22(7): 1299-1309), AAV1 R6, AAV1 R7 (Albright et al., Mol Ther. 2018; 26(2): 510), rAAVrh.8 (Yang, et al., supra), AAV-BR1 (Marchio et al., EMBO Mol Med. 2016; 8(6): 592), AAV-PHP.S (Chan et al., Nat Neurosci. 2017; 20(8): 1 172), AAV-PHP.B (Deverman et al., Nat Biotechnol. 2016; 34(2): 204), and AAV-PPS (Chen et al., Nat Med. 2009; 15: 1215). The PHP.eB capsid differs from AAV9 such that, using AAV9 as a reference, the sequence DGTLAVPFK (SEQ ID NO: 41) is inserted between amino acids residues 586 and 587 of AAV9.

In some embodiments, AAV comprises AAV type 1 (AAV1), AAV type 2 (AAV2), AAV type 3 (including types AAV3A and AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), and AAV type 11 (AAV11) and any other AAV now known or later discovered.

Agents that Induce Cell Death or Inflammatory Response

Cell death can be classified according to the morphological appearance of the lethal process (that may be apoptotic, necrotic, autophagic or associated with mitosis), enzymological criteria (with and without the involvement of nucleases or distinct classes of proteases, like caspases), functional aspects (programmed or accidental, physiological or pathological) or immunological characteristics (immunogenic or non-immunogenic) (Kroemer et al., 2009).

Agents that induce cell (e.g., cancer cell) death, also referred to herein as “cell death stimulating agents” may be immunogenic or non-immunogenic in nature.

As used herein, the term “immunogenic cell death” or “immunogenic apoptosis” refers to dying cells that alert the immune system, which then mounts a therapeutic anti-cancer immune response and contributes to the eradication of residual tumor cells. Conversely, when cancer cells succumb to a non-immunogenic death modality, i.e., non-immunogenic cell death, they fail to elicit such a protective immune response.

As used herein, the term “anti-cancer immune response” refers to when an immune response is directed against tumor cells, in particular cancerous cells. The anti-cancer immune response is allowed by a reaction from the immune system of the subject to the presence of cells, preferably of tumor cells, dying from an immunogenic cell death (as defined previously).

As used herein, the terms “agent that induces an immunogenic cell death” or “immunogenic cell death stimulating agent” refer to an agent that induces cell death which then in turn induces an anti-cancer immune response. In some embodiments, the antibody or antigen binding fragment thereof, e.g., nanobody, to PMEPA-1 may target and/or transport one or more immunogenic cell death stimulating agents (e.g., an agent that induces an immunogenic cell death, e.g., chemotherapeutic agent or CAR T cells) which can help stimulate immune responses. In some embodiments, immunogenic cell death stimulating agents boost immune responses by activating APCs to enhance their immunostimulatory capacity. In some embodiments, immunogenic cell death stimulating agents boost immune responses by amplifying lymphocyte responses to specific antigens. In some embodiments, immunogenic cell death stimulating agents boost immune responses by inducing the local release of mediators, such as cytokines from a variety of cell types. In some embodiments, the immunogenic cell death stimulating agents suppress or redirect an immune response. In some embodiments, the immunogenic cell death stimulating agents induce regulatory T cells. In some embodiments, the immunogenic cell death stimulating agents increase the levels or activity of intra-tumoral T cells.

As used herein, the term “agent that induces a non-immunogenic cell death” refers to an agent that induces cell death, but fails to elicit a corresponding protective immune response in doing so.

In some embodiments, the term “agent that induces an inflammatory response” refers to an agent that induces an inflammatory response which in turn induces a pro-inflammatory cytokine cascade. Cytokines activate immune cells such as T cells and macrophages, stimulating them to produce more cytokines resulting in so-called cytokine storms or cascades. In some embodiments, the agent that induces an inflammatory response is a TLR4 agonist or a GP-130 agonist.

In some embodiments, the agent is selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof.

In some embodiments, the antibody or antigen binding fragment thereof, e.g., nanobody, to PMEPA-1, and cell death stimulating agents or inflammatory response stimulating agents are coupled (e.g., covalently associated or within the same structure such as within a nanoparticle, or the antibody or antigen binding fragment thereof, e.g., nanobody, to PMEPA-1, is decorating the cell membrane of a CAR T cell). In some embodiments, a nanoparticle comprises a lipid membrane (e.g., lipid bilayer, lipid monolayer, etc.), wherein at least one type of cell death stimulating agent is associated with the lipid membrane of the nanoparticle and at least one antibody or antigen binding fragment thereof, e.g., nanobody, to PMEPA-1, is associated with the lipid membrane of the nanoparticle. In some embodiments, at least one type of cell death stimulating agent is embedded within the lipid membrane of the nanoparticle and at least one antibody or antigen binding fragment thereof, e.g., nanobody, to PMEPA-1, is embedded within the lipid membrane of the nanoparticle. In some embodiments, the at least type of cell death stimulating agent is encapsulated by the lipid membrane of the nanoparticle and the at least one antibody or antigen binding fragment thereof, e.g., nanobody, to PMEPA-1, is associated and/or embedded in the lipid membrane or the nanoparticle.

In some embodiments, the at least one type of cell death stimulating agent, e.g., immunogenic or non-immunogenic cancer cell death stimulating agent, is associated with the interior surface of the lipid membrane of the nanoparticle and the antibody or antigen binding fragment thereof, e.g., nanobody, to PMEPA-1, is associated with the exterior surface of the lipid membrane of the nanoparticle. In some embodiments, the at least one type of cell death stimulating agent may be located at multiple locations of a nanoparticle. For example, a first type of cell death stimulating agent may be embedded within a lipid membrane, and a second type of cell death stimulating agent may be encapsulated within the lipid membrane of a nanoparticle. To give another example, a first type of cell death stimulating agent may be associated with the exterior surface of a lipid membrane, and a second type of cell death stimulating agent may be associated with the interior surface of the lipid membrane of a nanoparticle. In some embodiments, a first type of cell death stimulating agent may be embedded within the lipid bilayer of a nanoparticle, and the lipid bilayer may encapsulate a polymeric matrix throughout which a second type of cell death stimulating agent is distributed. In some embodiments, a first type of cell death stimulating agent and a second type of cell death stimulating agent may be in the same locale of a nanoparticle (e.g., they may both be associated with the exterior surface of a nanoparticle; they may both be encapsulated within the nanoparticle; etc.). One of ordinary skill in the art will recognize that the preceding examples are only representative of the many different ways in which multiple cell death stimulating agents or inflammatory response stimulating agents may be associated with different locales of nanoparticles. Multiple cell death stimulating agents or inflammatory response stimulating agents may be located at any combination of locales of nanoparticles.

In certain embodiments, cell death stimulating agents or inflammatory response stimulating agents may be interleukins, interferon, cytokines, etc. In specific embodiments, cell death stimulating agent may be a natural or synthetic agonist for a Toll-like receptor (TLR). In specific embodiments, nanoparticles incorporate a ligand for toll-like receptor (TLR)-7, such, as CpGs, which induce type I interferon production. In specific embodiments, cell death stimulating agent may be an agonist for the DC surface molecule CD40. In certain embodiments, to stimulate immunity rather than tolerance, a nanoparticle incorporates a cell death stimulating agent that promotes DC maturation (needed for priming of naive T cells) and the production of cytokines, such as type I interferons, which promote antibody responses and anti-viral immunity. In some embodiments, cell death stimulating agent may be a TLR-4 agonist, such as bacterial lipopolysaccharide (LPS), VSV-G, and/or HMGB-1. In some embodiments, cell death stimulating agent are cytokines, which are small proteins or biological factors (in the range of 5 kD-20 kD) that are released by cells and have specific effects on cell-cell interaction, communication and behavior of other cells. In some embodiments, cell death stimulating agent may be proinflammatory stimuli released from necrotic cells (e.g., urate crystals). In some embodiments, cell death stimulating agents or inflammatory response stimulating agents may be activated components of the complement cascade (e.g., CD21, CD35, etc.). In some embodiments, cell death stimulating agents or inflammatory response stimulating agents may be activated components of immune complexes. The cell death stimulating agents include TLR-1, TLR-2, TLR-3, TLR-4, TLR-5, TLR-6, TLR-7, TLR-8, TLR-9, and TLR-10 agonists. The inflammatory response stimulating agents include, but are not limited to, TLR-4 agonist and GP-130 agonist. The cell death stimulating agents also include complement receptor agonists, such as a molecule that binds to CD21 or CD35. In some embodiments, the complement receptor agonist induces endogenous complement opsonization of the nanocarrier. In some embodiments, the cell death stimulating agents also include cytokine receptor agonists, such as a cytokine. In some embodiments, the cytokine receptor agonist is a small molecule, antibody, fusion protein, or aptamer.

In some embodiments, there are more than one type of cell death stimulating agent, e.g., immunogenic and/or non-immunogenic cancer cell death stimulating agent. In some embodiments, the different cell death stimulating agents or different inflammatory response stimulating agents each act on a different pathway. The cell death stimulating agents, therefore, can be different Toll-like receptors, a Toll-like receptor and CD40, a Toll-like receptor and a component of the inflammasome, etc.

In some embodiments, the cell death stimulating agents or inflammatory response stimulating agents may be an adjuvant. Thus, in some embodiments, the present invention provides pharmaceutical compositions comprising nanoparticles formulated with one or more adjuvants. The term “adjuvant”, as used herein, refers to an agent that does not constitute a specific antigen, but boosts the immune response to the administered antigen.

In some embodiments, the present invention is directed to delivery of adjuvant using nanoparticles capable of carrying the adjuvant (encapsulated and/or on a surface) to targeted locations such as a tumor vascular endothelial cell, wherein the nanoparticle comprises: (i) one or more molecules on a surface to target a specific cell; (iii) one or more molecules that are capable of eliciting a cell death when covalently attached to a polymer or encapsulated inside the nanoparticles. The embodiment is directed to enhancing the potentiating of an immune response in a mammal, comprising administering art effective amount of a nanoparticle delivery of adjuvant of the present invention to enhance the immune response of a mammal to one or more antigens.

For example, in some embodiments, the adjuvant is encapsulated within the nanoparticles of the invention. Typically, in such cases, the adjuvant is present in free form, i.e., the adjuvant is not conjugated to the polymers that form the nanoparticles. Adjuvant is encapsulated during the preparation of the nanoparticles in the usual manner, as exemplified herein. The release profile of the adjuvant from the nanoparticles when administered to a patient will depend upon a variety of factors, including the size of the nanoparticles, rate of dissolution of the polymer forming the nanoparticles (if dissolution occurs), the molecular weight of the polymer forming the nanoparticles, and the chemical characteristics of the adjuvant (which, in turn, will influence the location of the adjuvant within the nanoparticles, diffusion rates, etc.). The amount of adjuvant encapsulated in the polymer nanoparticles will be determined during the process of formation of the nanoparticles.

For example, in some embodiments, the adjuvant is conjugated to the polymers that form the nanoparticles. Typically, in such cases, the adjuvant is expressed on or near the surface of the nanoparticles. In some embodiments, an amphilic polymer capable of self-assembling into nanoparticles is used, and the adjuvant is covalently attached to one terminus of the polymer. For example, the adjuvant may be used as an initiating species in the polymerization reaction used to form the polymers. When the adjuvant is conjugated (i.e., covalently bonded) to a terminus of the polymer, upon self-assembly of the polymer, the adjuvant is concentrated at the periphery or at the core of the nanoparticles. For example, in a polymer comprising a hydrophobic block and a hydrophilic block, wherein the polymer is allowed to self-assemble into nanoparticles having a hydrophobic core and a hydrophilic periphery, adjuvant that is conjugated to the terminus of the hydrophilic block will be concentrated at the periphery of the nanoparticles. In some preferred embodiments, the adjuvant is concentrated at the surface of the nanoparticles and remains in a position to act as an immunostimulant. For nanoparticle formulations comprising conjugated adjuvant, the density of adjuvant on the surface of the nanoparticles will be a function of a variety of factors, including the molecular weight of the polymers forming the nanoparticles, the density of the nanoparticles, and the chemical characteristics of the adjuvant.

In some embodiments, a combination of encapsulated and conjugated adjuvant is used. For example, in some embodiments, nanoparticles are formulated with one or more adjuvants such as gel-type adjuvants (e.g., aluminum hydroxide, aluminum phosphate, calcium phosphate, etc.), microbial adjuvants (e.g., immunomodulatory DNA sequences that include CpG motifs; endotoxins such as monophosphoryl lipid A; exotoxins such as cholera toxin, E. coli heat labile toxin, and pertussis toxin; muramyl dipeptide, etc.); oil-emulsion and emulsifier-based adjuvants (e.g., Freund's Adjuvant, MF59 [Novartis], SAF, etc.); particulate adjuvants (e.g., liposomes, biodegradable microspheres, saponins, etc.); synthetic adjuvants (e.g., nonionic block copolymers, muramyl peptide analogues, polyphosphazene, synthetic polynucleotides, etc.), surfactant based adjuvants, and/or combinations thereof. Other exemplary adjuvants include some polymers (e.g., polyphosphazenes, described in U.S. Pat. No. 5,500,161, which is incorporated herein by reference), QS21, squalene, tetrachlorodecaoxide, etc.

The term “adjuvant” is intended to include any substance which is incorporated into or administered simultaneously with the conjugates of the invention and which nonspecifically potentiates the immune response in the subject. Adjuvants include aluminum compounds, e.g., gels, aluminum hydroxide and aluminum phosphate; and Freund's complete or incomplete adjuvant (in which the conjugate is incorporated in the aqueous phase of a stabilized water in paraffin oil emulsion). The paraffin oil may be replaced with different types of oils, e.g., squalene or peanut oil. other materials with adjuvant properties include BCG (attenuated Mycobacterium tuberculosis), calcium phosphate, levamisole, isoprinosine, polyanions (e.g., poly A:U) leutinan, pertussis toxin, choler toxin, lipid A, saponins and peptides, e.g., muramyl dipeptide. Rare earth salts, e.g., lanthanum and cerium, may also be used as adjuvants. The number and/or amount of adjuvants depends on the subject and the particular conjugate used and can be readily determined by one skilled in the art without undue experimentation. The adjuvant to be incorporated in the nanoparticle system and delivered to a target cell or tissue of the present invention may be combined with a diagnostic, antigen, prophylactic or prognostic agents. Any chemical compound to be administered to an individual may be delivered using the adjuvant nanoparticle delivery system of the invention.

In certain embodiments, a lipid to be used in nanoparticle can be, but is not limited to, one or a plurality of the following: phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and lyso-phophatides. In certain embodiments, an immunomodulatory agent can be conjugated to the surface of a nanoparticle. In some embodiments, the nanoparticle surface membrane can be modified with the antibody or antigen binding fragment thereof, e.g., nanobody, to PMEPA-1, that can selectively deliver the cell death stimulating agent(s), e.g., immunogenic or non-immunogenic cell death stimulating agent, to specific transmembrane expressing cells (e.g., tumor vascular endothelial cells).

### CAR T Cells

In some embodiments, the cell death stimulating agent is a chimeric antigen receptor T cell (CAR T cell).

As used herein, a “chimeric antigen receptor” (CAR) is an artificially constructed hybrid protein or polypeptide comprising a specificity or recognition (i.e. binding) domain linked to an immune receptor responsible for signal transduction in lymphocytes. The binding domain is typically derived from a Fab antibody fragment that has been fashioned into a single chain scFv via the introduction of a flexible linker between the antibody chains within the specificity domain. Other possible specificity domains can include the signaling portions of hormone or cytokine molecules, the extracellular domains of receptors, and peptide ligands or peptides isolated by library (e.g. phage) screening (see Ramos and Dotti, (2011) Expert Opin Bio Ther 11(7): 855). Flexibility between the signaling and the binding portions of the CAR may be a desirable characteristic to allow for more optimum interaction between the target and the binding domain, so often a hinge region is included. One example of a structure that can be used is the CH2-CH3 region from an immunoglobulin such as an IgG molecule. The signaling domain of the typical CAR comprises intracellular domains of the TCR-CD3 complex such as the zeta chain. Alternatively, the y chain of an Fe receptor may be used. The transmembrane portion of the typical CAR can comprise transmembrane portions of proteins such as CD4, CD8 or CD28 (Ramos and Dotti, ibid). Characteristics of some CARs include their ability to redirect T-cell specificity and reactivity toward a selected target in a non-MHC-restricted manner. The non-MHC-restricted target recognition gives T-cells expressing CARs the ability to recognize a target independent of antigen processing, thus bypassing a major mechanism of tumor escape.

In some embodiments, the surface of the CAR T cells is decorated with one or more antibody or antigen binding fragment thereof such as a nanobody, to PMEPA-1. In some embodiments, the antibody or antigen binding fragment thereof, e.g., nanobody, to PMEPA-1, is embedded in the lipid membrane of the CAR T cell. In some embodiments, the antibody or antigen binding fragment thereof, e.g., nanobody, to PMEPA-1, is associated with the lipid membrane of the CAR T cell (e.g., binding to molecule on the exterior of the CAR T cell, covalently linked to a molecule on the exterior of the CAR T cell). In some embodiments, there are two or more different types of targeting molecules, one of which is an antibody or antigen binding fragment thereof, e.g., nanobody, to PMEPA-1 and, optionally, at least one targeting molecule to an additional transmembrane molecule from Tables 2-4, on the exterior of the CAR T cells.

### Treatment of Disease or Disorders

As used herein, the term “therapeutically effective amount” means an amount of a therapeutic, prophylactic, and/or diagnostic agent (e.g., inventive vaccine nanocarrier) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. The term is also intended to refer to an amount of nanocarrier or composition thereof provided herein that modulates an immune response in a subject.

As used herein, the term “therapeutic agent” refers to any agent that, when administered to a subject, has a therapeutic, prophylactic, and/or diagnostic effect and/elicits a desired biological and/or pharmacological effect.

As used herein, the term “treating” refers to a partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive vaccine nanocarrier to a subject.

In some embodiments, the disease or disorder is a cancer. In some embodiments, the cancer is a non-immunogenic cancer. In some embodiments, the cancer is a hematological cancer. In some embodiments, the cancer is a solid tumor. In some embodiments, the cancer is melanoma, pancreatic cancer, and colorectal cancer.

In some embodiments, the disease or disorder the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, e.g., a biomarker, the presence, absence, amount, or change in amount of which is indicative of the presence, severity, or absence of the condition.

As used herein the term “prognosis” shall be taken to mean an indicator of the predicted progression of the disease (including but not limited to aggressiveness and metastatic potential) and/or predicted patient survival time.

As used herein, the term “identifying” or grammatical variations thereof refer to determining the presence of a diagnostic indicators, e.g., tumor vascular endothelial cells expressing one or more transmembrane molecules, e.g., PMEPA-1 and/or another transmembrane molecule from Tables 2-4, wherein the one or more transmembrane molecules, e.g., PMEPA-1 and/or another transmembrane molecule from Tables 2-4, are upregulated when compared to a non-diseased control.

The term “control” or “control sample,” as used herein, refers to any clinically relevant control sample, including, for example, a sample from a healthy subject not afflicted with the disease or condition being assayed (e.g., cancer), a sample from a subject having a less severe or slower progressing disease or condition (e.g., cancer) than the subject to be assessed, a sample from a subject having some other type of cancer or disease, and the like. A control sample may include a sample derived from one or more subjects. A control sample may also be a sample made at an earlier timepoint from the subject to be assessed. For example, the control sample could be a sample taken from the subject to be assessed before the onset of the disease or condition being assayed (e.g., cancer), at an earlier stage of disease, or before the administration of treatment or of a portion of treatment. The control sample may also be a sample from an animal model, or from a tissue or cell lines derived from the animal model, of the disease or condition being assayed (e.g., cancer). For example, the expression level of a molecule, such as PMEPA-1, in a control sample that consists of a group of measurements may be determined based on any appropriate statistical measure, such as, for example, measures of central tendency including average, median, or modal values.

The term “control level” refers to an accepted or pre-determined expression level of a molecule, such as PMEPA-1 which is used to compare with the expression level of a molecule, such as PMEPA-1 in a sample derived from a subject. In one embodiment, the control level of a molecule, such as PMEPA-1 is based on the expression level of the molecule in sample(s) from a subject(s) having slow disease progression. In another embodiment, the control level of the molecule, such as PMEPA-1, is based on the expression level in a sample from a subject(s) having rapid disease progression. In another embodiment, the control level of the molecule, such as PMEPA-1, in based on sample(s) from an unaffected, i.e., non-diseased, subject(s), i.e., a subject who does not have a disease or disorder (e.g., cancer). In yet another embodiment, the control level of the molecule, such as PMEPA-1, is based on the expression level of the molecule in a sample from a subject(s) prior to the administration of a therapy for the disease or disorder (e.g., cancer). In yet another embodiment, the control level of the molecule, such as PMEPA-1, is based on the expression level of the molecule in a sample from a subject(s) after the administration of a therapy for the disease or disorder (e.g., cancer). In one embodiment, the control level of the molecule, such as PMEPA-1, is based on the level in a sample(s) from an animal model of a disease or disorder, (e.g., cancer), a cell, or a cell line derived from the animal model of a disease or disorder, (e.g., cancer).

In some embodiments, the disclosure provides methods for treating a subject having cancer including the use of a composition comprising an antibody or antigen binding fragment thereof, e.g., nanobody, to PMEPA-1. In some embodiments, the method involves further administering a targeting molecule that binds a transmembrane molecule on a tumor vascular endothelial cell selected from Tables 2-4. In some embodiments, the tumor vascular endothelial cell is a cancer cell and/or is a venular cell. In some embodiments, PMEPA-1 is upregulated in the tumor vascular endothelial cell when compared to a non-tumor vascular endothelial control cell. In some embodiments, the composition further comprises an agent that induces cell death, e.g., an agent that induces immunogenic or non-immunogenic cancer cell death, in the tumor vascular endothelial cell expressing the target transmembrane molecule.

In some embodiments, the agent that induces cell death is a chemotherapeutic agent or a CAR T cell. In some embodiments, the agent is a CAR T cell.

In some embodiments, the methods of use provided herein include identifying if a subject has tumor vascular endothelial cells derived from a tumor that express PMEPA-1 and, optionally, one or more transmembrane molecules from Tables 2-4. In some embodiments, the method comprises administering an antibody or antigen binding fragment thereof, e.g., nanobody, to PMEPA-1, coupled to a diagnostic agent to determine if a subject has tumor vascular endothelial cells that express PMEPA-1. In some embodiments, the method comprises determining whether PMEPA-1 is upregulated in comparison to a control cell. In some embodiments, the method comprises determining whether PMEPA-1 is downregulated in comparison to a control cell.

In some embodiments, the methods of use provided herein include determining if a treatment of cancer in a subject is effective. In some embodiments, the antibody or antigen binding fragment thereof, e.g., nanobody, to PMEPA-1, is used to determine the presence of tumor vascular endothelial cells expressing PMEPA-1 before treatment, after treatment and compared to a non-tumor vascular endothelial cell. In some embodiments, the level of tumor vascular endothelial cells expressing PMEPA-1 is decreased after treatment in comparison to the level prior to administration of the treatment indicating the treatment is effective. In some embodiments, the level of tumor vascular endothelial cells expressing PMEPA-1 after treatment is increased or stays the same in comparison to the level of tumor vascular endothelial cells expressing PMEPA-1 prior to administration of the treatment indicating the treatment is not effective.

In some embodiments, the methods of use provided herein include modifying gene expression of PMEPA-1, wherein PMEPA-1 is upregulated in comparison to a control cell.

In some embodiments, the tumor vascular endothelial cell is contacted with a composition comprising an antibody or antigen binding fragment thereof, e.g., nanobody, to PMEPA-1, and a nucleic acid capable of modifying the expression of said transmembrane molecule.

In some embodiments, the nucleic acid capable of modifying the expression of PMEPA-1 encodes an inhibitory RNA molecule or a CRISPR-Cas9 system.

In certain circumstances, it will be desirable to deliver the agent that induces cell death, e.g., an agent that induces immunogenic or non-immunogenic cancer cell death, and the antibody or antigen binding fragment thereof, e.g., nanobody, to PMEPA-1 in suitably formulated compositions disclosed herein either by pipette, retro-orbital injection, subcutaneously, intraocularly, intravitreally, parenterally, subcutaneously, intravenously, intracerebroventricular (ICV), intravenous injection into the cisterna magna (ICM), intracerebro-ventricularly, intramuscularly, intrathecally, intraspinally, orally, intraperitoneally, by oral or nasal inhalation, or by direct application or injection to one or more cells, tissues, or organs.

In some embodiments, the antibody or antigen binding fragment thereof, e.g., nanobody, to PMEPA-1, is associated with a nanoparticle comprising nucleic acids capable of altering gene expression in a cell.

As used herein, the term “gene” may include not only coding sequences but also regulatory regions such as promoters, enhancers, and termination regions. The term further can include all introns and other DNA sequences spliced from the mRNA transcript, along with variants resulting from alternative splice sites. The term further refers to a coding sequence for a desired expression product of a polynucleotide sequence such as a polypeptide, peptide, protein or interfering RNA including short interfering RNA (siRNA), miRNA or small hairpin RNA (shRNA). The sequences can also include degenerate codons of a reference sequence or sequences that may be introduced to provide codon preference in a specific organism or cell type. As used herein, the term “heterologous gene” refers to a gene provided to the target cell by an exogenous source, such as a viral vector, e.g., rAAV. In some embodiments, the gene encodes a polypeptide or a nucleic acid molecule, such as microRNA (miRNA), artificial microRNA (amiRNA), and short hairpin RNA (shRNA).

### Formulations

Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in some embodiments, when administered intravenously (e.g., at the retro-orbital plexus).

In some embodiments, compositions can be formulated for intravenous, intraocular, intravitreal, parenteral, subcutaneous, intracerebro-ventricular, intramuscular, intracerebroventricular, intravenous injection into the cisterna magna (ICM), intrathecal, intraspinal, oral, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

As used herein, the term “lipid nanoparticle” refers to a vesicle formed by one or more lipid components. Lipid nanoparticles are typically used as carriers for nucleic acid delivery in the context of pharmaceutical development. They work by fusing with a cellular membrane and repositioning its lipid structure to deliver a drug or active pharmaceutical ingredient (API). Generally, lipid nanoparticle compositions for such delivery are composed of synthetic ionizable or cationic lipids, phospholipids (especially compounds having a phosphatidylcholine group), cholesterol, and a polyethylene glycol (PEG) lipid; however, these compositions may also include other lipids. The sum composition of lipids typically dictates the surface characteristics and thus the protein (opsonization) content in biological systems thus driving biodistribution and cell uptake properties.

As used herein, the “liposome” refers to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “ionizable lipid” refers to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. Ionizable lipids are also referred to as cationic lipids herein.

As used herein, the term “non-cationic lipid” refers to any amphipathic lipid as well as any other neutral lipid or anionic lipid. Accordingly, the non-cationic lipid can be a neutral uncharged, zwitterionic, or anionic lipid.

As used herein, the term “conjugated lipid” refers to a lipid molecule conjugated with a non-lipid molecule, such as a PEG, polyoxazoline, polyamide, or polymer (e g., cationic polymer).

As used herein, the term “excipient” refers to pharmacologically inactive ingredients that are included in a formulation with the API, e.g., ceDNA and/or lipid nanoparticles to bulk up and/or stabilize the formulation when producing a dosage form. General categories of excipients include, for example, bulking agents, fillers, diluents, antiadherents, binders, coatings, disintegrants, flavours, colors, lubricants, glidants, sorbents, preservatives, sweeteners, and products used for facilitating drug absorption or solubility or for other pharmacokinetic considerations.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24(12): 11 13-1 128, 1998; Quintanar-Guerrero et al, Pharm Res. 15(7): 1056-1062, 1998; Quintanar-Guerrero et al., J. Microencapsul. 15(1): 107-1 19, 1998; Douglas et al, Crit Rev Ther Drug Carrier Syst 3(3):233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69(2): 199-202, 1980; Couvreur et al., Crit Rev Ther Drug Carrier Syst. 5(1)1-20, 1988; zur Muhlen et al., EurJ Pharm Biopharm, 45(2): 149-155, 1998; Zambau x et al., J Control Release 50(1-3):31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In some embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al, Prog Retin Eye Res, 17(1):33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions. Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In some embodiments, for administration to humans, compositions should meet sterility, pyrogenicity, and the general safety and purity standards as required by United States Food and Drug Administration (FDA) or other applicable regulatory agencies in other countries.

### EXAMPLES

### Example 1: Characterize PMEPA-1 Expression on Microvascular ECs of Murine and Human Solid Tumors

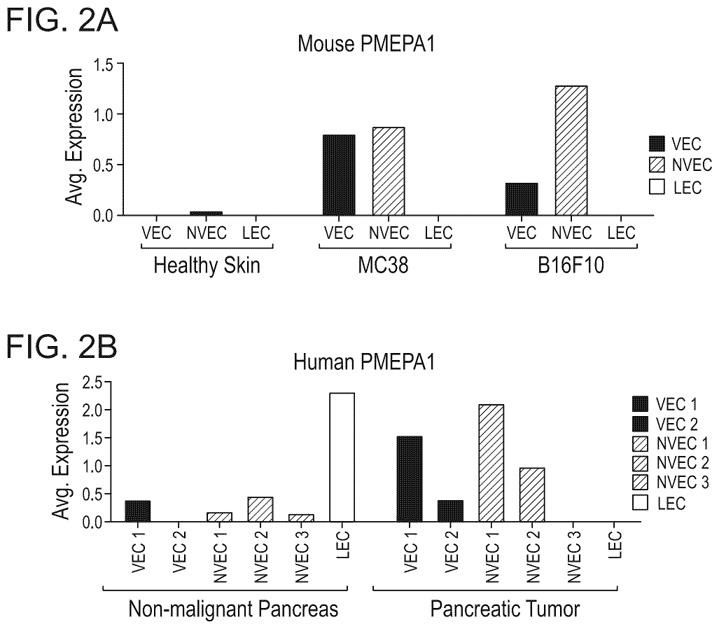

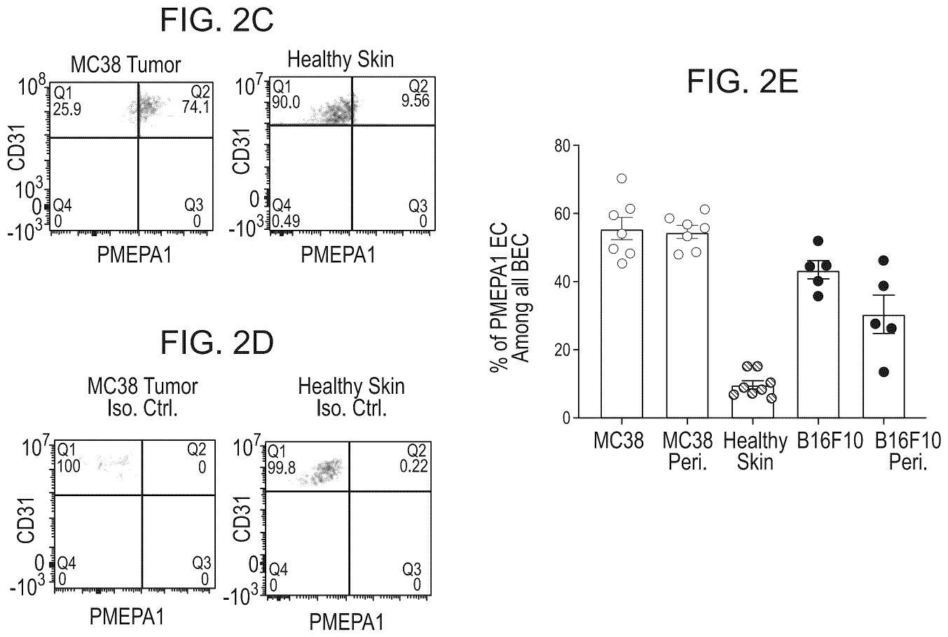

To determine PMEPA-1 mRNA and protein expression and localization in primary human and murine tumors qPCR, FISH, flow cytometry and histology were be used. For murine tumor models, syngeneic colorectal adenocarcinoma (MC38) and melanoma (B16F10) tumor models were used, whereby tumor cells are implanted subcutaneously in the dorsal skin. MC38 is an immunogenic tumor with high T cell infiltrates (TILs) and responds to checkpoint blockade while B16F10 has the opposite characteristics. In FACS analysis of ECs from subcutaneously implanted syngeneic murine MC38 and B16F10 tumors, 74.1% of intratumoral ECs were PMEPA-1+, whereas only 9.6% of ECs in normal skin expressed PMEPA-1, and only at a very low level (FIGS. 2A-2E). For FACS analysis, the gating strategy for PMEPA-1+ events was based on a polyclonal control antibody (Iso). The apparent basal level of PMEPA-1 in healthy murine skin could reflect high background binding that is typical for many polyclonal Abs rather than true protein expression (FIGS. 2A-2E). Indeed, healthy skin does not appear to express PMEPA-1 at the mRNA level (FIG. 2A). PMEPA-1 expression in fresh samples of human melanoma and peri-tumoral non-malignant skin, as well as, human pancreatic cancer and non-malignant pancreas at the protein level by flow cytometry and IHC were accessed.

### Example 2: Yeast Display sdAb Library to Generate and Validate sdAbs Against the PMEPA-1 Ectodomain

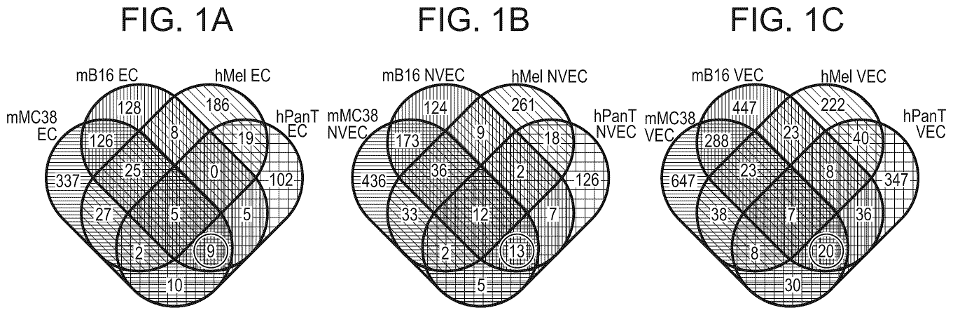

Among these tumor EC restricted genes is Prostate Transmembrane Protein, Androgen Induced 1 (PMEPA-1), a regulator of tissue responses to cytokines. PMEPA-1 mRNA was significantly upregulated in ECs from MC38, B16F10 and human pancreatic cancer samples compared to ECs in nonmalignant peri-tumoral tissue (FIGS. 1A-1C and 2A-2E). PMEPA-1 is also upregulated in human melanoma compared to healthy skin, however, it did not reach statistical significance in the analysis (red circles in FIGS. 1A-1C). Additionally, PMEPA-1 is present in 17 types of solid human tumors according to the TCGA database. Although EC expression of PMEPA-1 in these tumors remains to be validated, it appears to be an attractive candidate to target ECs in a wide variety of tumors.

PMEPA-1 mRNA and protein expression levels in normal and malignant human and murine tissues was validated. Mouse and human PMEPA-1 have a single transmembrane domain and an ectodomain with 79% aa homology. While there are no reagents that specifically recognize this ectodomain, a commercially available polyclonal antibody to the cytoplasmic tail was used to validate at the protein level that PMEPA-1 is preferentially expressed on tumor ECs (FIGS. 2A-2E).

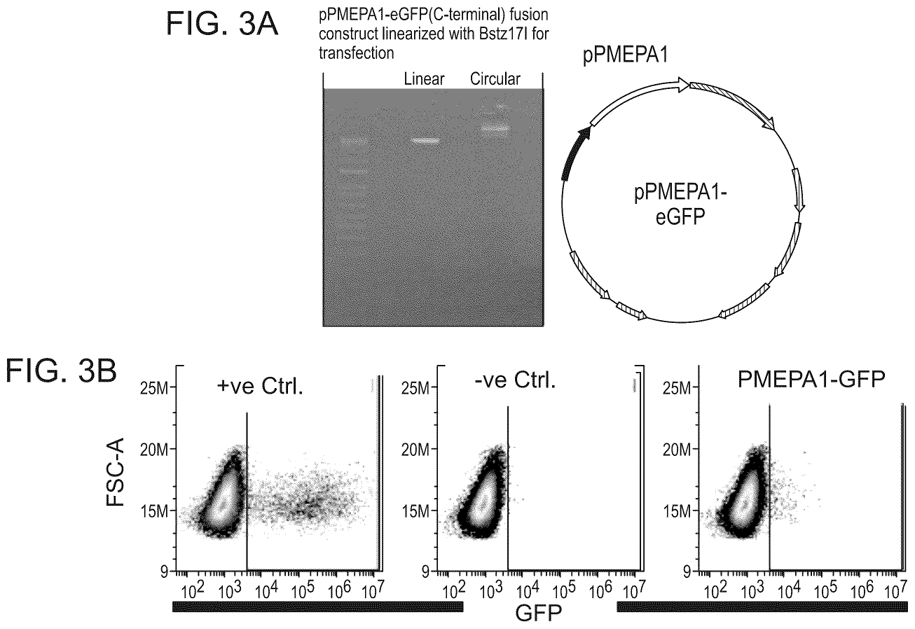

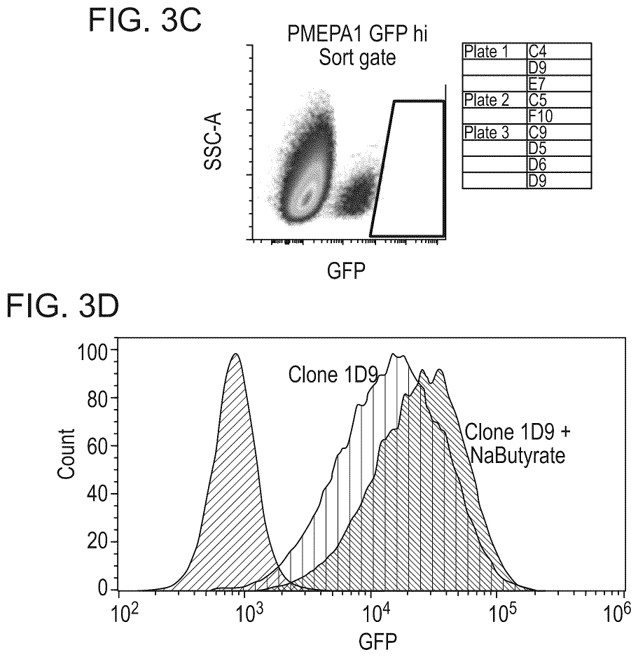

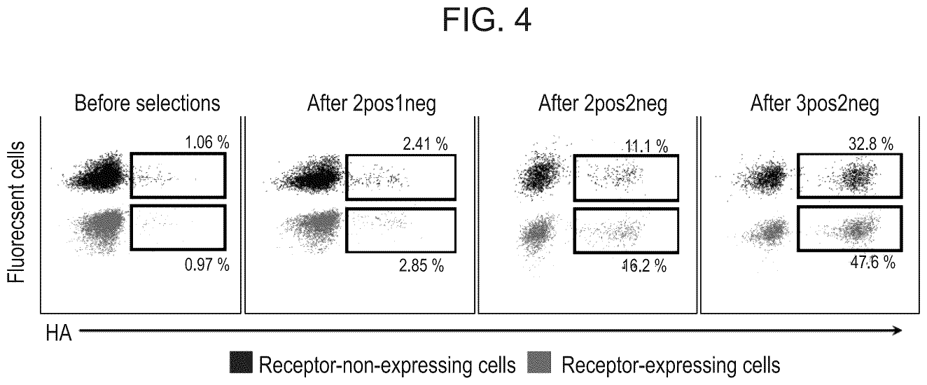

A yeast display library was used to raise sdAbs against human and murine PMEPA-1 transfectants (FIGS. 3A-3D). Stably transfected L1.2 cells that express PMEPA-1 fused with intracellular GFP were used. As negative and positive controls, L1.2 cells were transfected with empty vector or GFP alone (FIGS. 3A-3B). PMEPA-1-GFP high cells were FACS sorted and subcloned by limiting dilution in 96-well plates and expanded in selection medium containing G418 (FIG. 3C). Clones which displayed consistently the highest mean fluorescence intensity were further expanded. Clone 1D9 that demonstrated the highest PMEPA-1-GFP expression, which could be further enhanced by addition of sodium butyrate, an HDAC inhibitor, to the culture medium was selected (FIG. 3D). This clone is ready for use as ‘bait’ for the yeast display library to identify sdAb with reactivity against the ectodomain of PMEPA-1. FIG. 4 shows an example where the yeast library was subjected alternatingly to three and two cycles of positive and negative selection, respectively. sdAb mediated yeast binding to target cells is readily detectable by FACS because sdAb expressing yeast cells coexpress a surface epitope from hemagglutinin (HA).

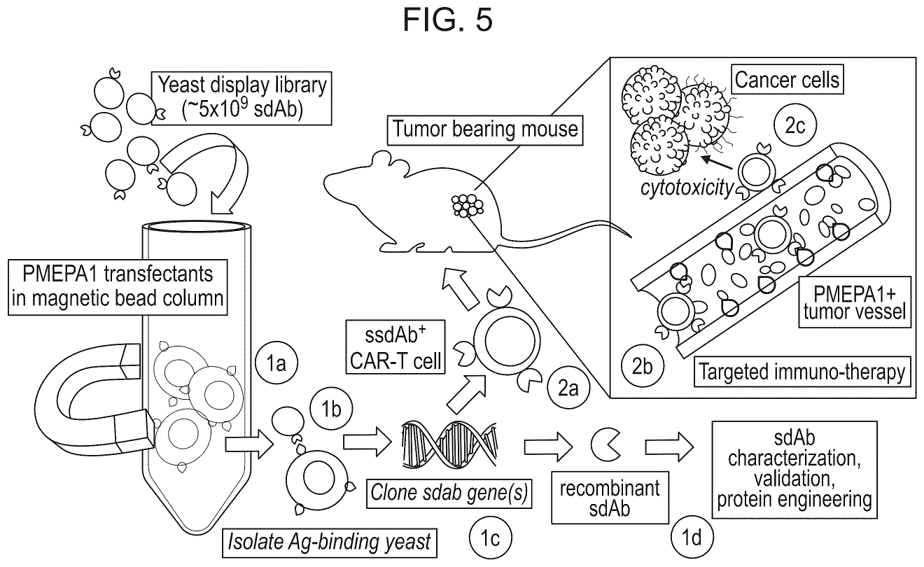

The yeast display library approach is based on performing a series of alternating magnetic-activated cell sorting (MACS)-based positive and negative selection steps followed by fluorescence-activating cell sorting (FACS)-based sorts (FIG. 5, Step 1a). For positive selections, PMEPA-1-GFP expressing L1.2 cells will be labeled with anti-CD45 magnetic beads and loaded on magnetic columns. Next, sdAb-expressing yeast (which contains ˜5×10⁹ distinct sdAb clones) will be loaded on the same columns, and columns will be washed extensively. The contents of the columns will be retrieved, and yeast bound to L1.2 cells (determined as HA+ cells) will be sorted. Thus, the clones expressing relevant sdAb will be enriched. Each positive selection is followed by a negative selection cycle, whereby PMEPA-1-negative L1.2 cells will be loaded on magnetic columns, followed by loading of sdAb-expressing yeast. The columns will be washed, and the unbound fraction will be collected. and HA+ yeast cells will be sorted. Yeast clones that bind to irrelevant surface antigens on L1.2 cells remain in the column and are eliminated (FIG. 5, Step 1b). Repeated cycles of positive and negative selection will result in decreased sdAb library diversity and increased efficiency of formation of cell-yeast conjugates, with enrichment for yeast clones that preferentially bind PMEPA-1-expressing L1.2 cells. Upon reaching a high frequency of cell-yeast conjugates, the yeast sdAb library will be subcloned (FIG. 5, Step 1c), and the clones with maximum binding to PMEPA-1-expressing L1.2 cells and no binding to control L1.2 cells will be identified. The sdAb encoding cDNAs will be subcloned into an expression vector and modified to append an N-terminal FLAG tag for protein purification and/or a C-terminal LPETG motif to allow for sortase A-mediated “click chemistry” linkage to acceptor moieties of interest. Recombinant sdAb will be expressed in E. coli and extensively characterized for reactivity with PMEPA-1 in vitro (FACS, Western blot) and in situ using IHC and/or intravital microscopy of microvessels in tumors and non-malignant tissues at various anatomical location (FIG. 5, Step 1d). In addition, sdAbs will be engineered to allow surface expression/immobilization on CAR-T cells to test their ability to selectively target tumor ECs (FIG. 5, Steps 2a-2c).

### Example 3: Target CAR-T Cells with PMEPA-1 sdAb to Tumor Microvessels and Assess Anti-Tumor Efficacy

Immobilized PMEPA-1 sdAb on the surface of CAR T cells will be used to determine if these CAR T cells can be used as an effective targeted cell therapy. After IV infusion, the sdAb will enable CAR T cells to adhere selectively to tumor ECs that are normally non-adhesive for circulating T cells. Because of the immobilized PMEPA-1 sdAb on the CAR T cells, it is expected that the CAR T cells will accumulate in solid tumors that are currently resistant to CAR T cell therapy. In particular, the lack of tumor targeting specificity of traditional CAR T cell therapies increases the risk for off-target effects and exhaustion. It is expected that sdAb-mediated targeting of such second-generation CAR T cells to tumors will further boost therapeutic efficacy.

The surface of the T cells that express a CAR specific for a tumor antigen will be decorated with PMEPA-1 sdAb at a high density. In this setting, the sdAb will confer mechanical stability to CAR T cell binding to PMEPA-1+ tumor microvessels, without transmitting an activating signal. As described above, at the transcriptional level, among all intravascular cells in mice only ECs lining the vasculature within tumors express robust levels of PMEPA-1. Indeed, according to RNASeq data published by Immgen, among all murine cell types tested, the only healthy cells that express PMEPA-1 are brain microglia (and, to a lesser degree, alveolar macrophages) (www.rstats.immgen.org/Skyline/skyline). After adoptive transfer, CAR T cell are unlikely to access the CNS because normal brain ECs do not express PMEPA-1 and do not support substantial T cell trafficking. Therefore, sdAb decoration should focus CAR T cells onto tumor ECs without redirecting them to other cell types or anatomic sites. In humans, according to the human cell atlas website (www.humancellatlas.org), only a subset of PBMCs (Siglec6-CD123+CD11c-PBMC) from healthy patients had mRNA levels slightly above baseline. Detection of PMEPA-1 has been reported only at the mRNA level in these databases, which does not always correlate with the presence of protein, especially if the RNA level is low. For the most rigorous analysis, a reliable antibody detecting the ectodomain of this transmembrane molecule is needed to allow for sensitive assessment by flow cytometry and immuno-histochemistry. Due to the absence of such a reagent, very few studies have been performed on this molecule to date.

Although it is expected that the sdAb-targeted CAR T cells will be highly selective for tumor ECs, there may be other PMEPA-1+ target cells. However, even if this were the case, expression of PMEPA-1 sdAb without a signaling domain should not cause increased toxicity as long as such hypothetical PMEPA-1+ cells do not also express the CAR antigen or other means to activate CAR T cells.

A schematic of the proposed protocol is shown in FIG. 5. Briefly, CAR-T cells express chimeric Ag receptors (CARs) that link an extracellular Ag recognition component to an intracellular signaling domain resulting in T cell activation when a tumor Ag is encountered. CAR T cells will be generated against human CD20. These CAR T cells will be modified to display one or more PMEPA-1 sdAbs on their surface by transfecting CAR T cells with chimeric sdAbs containing either a cytoplasmic and transmembrane domain and a linker region or a Gpi anchor (FIG. 5, Step 2a). The PMEPA-1 sdAb constructs will not include an activating signaling domain. The sdAb may function as an anchor to immobilize CAR T cells within tumor microvessels upon adoptive transfer into tumor bearing mice. Thus, adoptive transfer experiments and in situ imaging in tumor bearing mice will be performed to determine whether surface displayed sdAbs enhance CAR T cell accumulation in tumors (FIG. 5, Step 2b). MC38 or B16F10 solid tumors will be transduced to express human CD20 with anti-human CD20 CAR T cells that will be surface modified either with anti-PMEPA-1 sdAbs or a non-binding control sdAb. Assessment whether CAR T cell decoration with anti-PMEPA-1 sdAb confers selective targeting of CAR T cells to tumors and enhancement of anti-tumor immunity will be performed (i.e. suppression of tumor growth, survival etc.) (FIG. 5, Step 2c).

The anti-PMEPA-1 sdAb serving as the Ag binding domain of a CAR will also be tested to determine if T cell activation after recognition of PMEPA-1 by the sdAb-CAR results in tumor EC killing. In this setting, the CAR T cells would exert cytotoxic activity towards tumor ECs, destroying the tumor by going after these vital stromal cells rather than the tumor cells themselves. While this approach would likely result in rapid killing of host tumors since CAR T cells will initially be present at a high density in the blood stream, there may be a greater risk for on-target off-tumor side effects due to recognition of PMEPA-1 on cells other than intra-tumoral ECs. Thus, recipient animals' health and potential organ damage will be monitored. If off-target toxicity is unacceptable, it could offer a powerful new treatment modality for solid tumors because the CAR T cells could function within the tumor vessel lumen, without the need to extravasate.

### Example 4: Development of Nanobodies Against PMEPA-1

After identification of PMEPA-1 as a prime candidate gene that will allow specific targeting of tumor microvasculature, a yeast surface display nanobody (Nb) library was used to generate a panel of immunoreagents specific for PMEPA-1. (McMahon C, et al., Yeast surface display platform for rapid discovery of conformationally selective nanobodies. Nat Struct Mol Biol. 2018 25(3):298-296). Nanobodies, also known as single-domain antibodies or VHH, are small (˜15 kDa), stable proteins comprising a single immunoglobulin domain capable of binding specific target molecules with high affinity, and have the potential to bind epitopes that are less immunogenic to conventional antibodies. In addition, due to their small size, nanobodies can target areas that are not accessible to conventional antibodies, and can be easily employed as a modular component—i.e. coupled to other molecules (e.g. CAR platform, therapeutic agents, etc.) without loss of specificity or affinity.

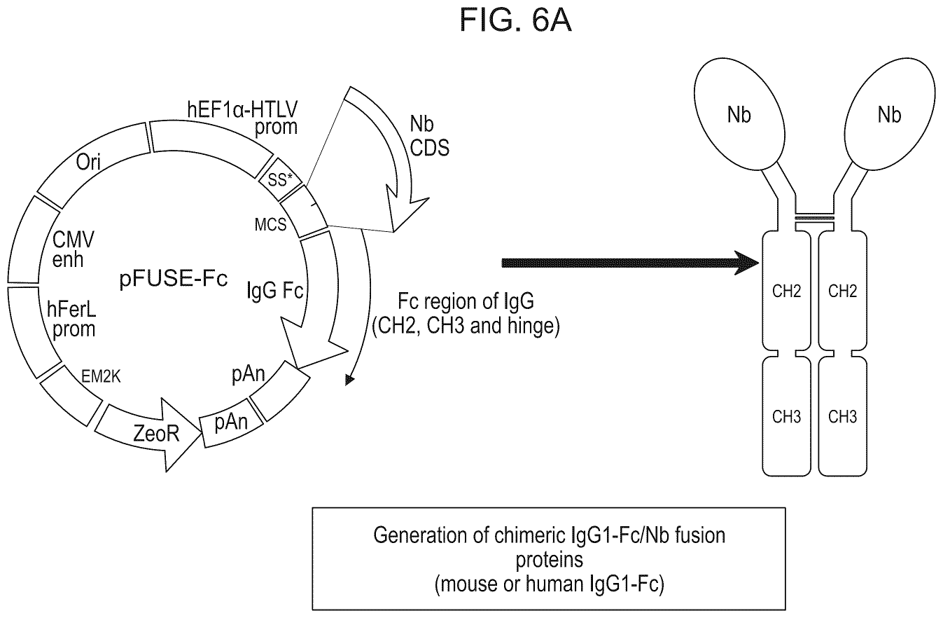

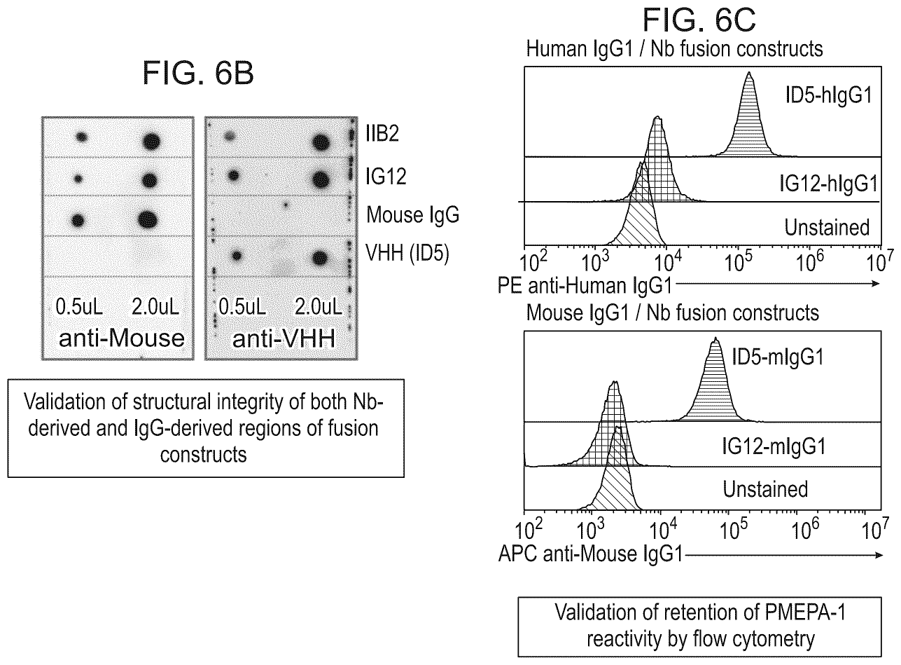

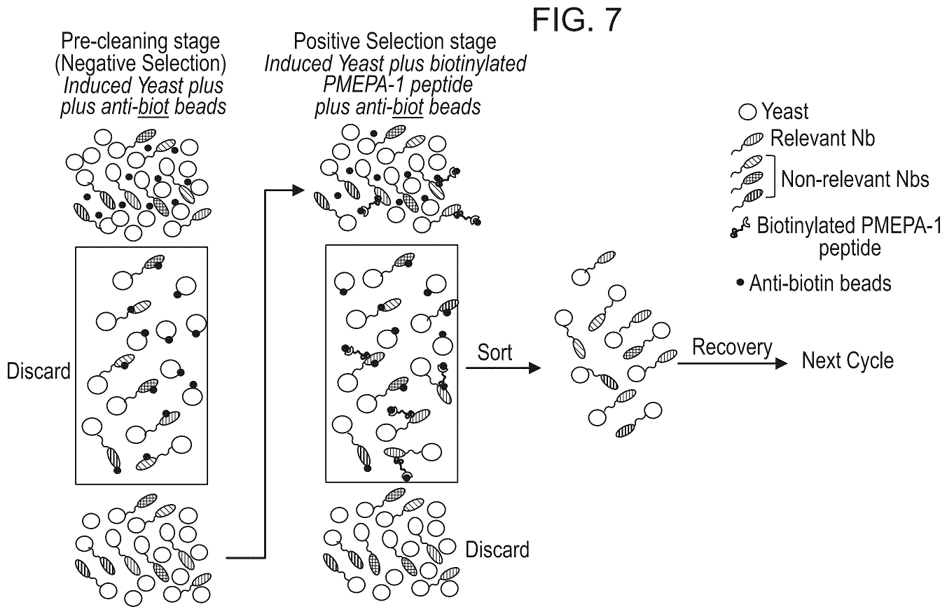

To generate the PMEPA-1 nanobody-IgG fusion constructs, the nanobody CDS were cloned into expression vector pFUSE-mIgG1-Fc2 or pFUSE-hIgG1-Fc2 (mouse and human IgG1 CH2-CH3 domains, respectively, downstream of the IL-2 signal sequence to promote efficient secretion, (InvivoGen, San Diego, CA)), generating chimeric proteins as pictured in FIG. 6A. Dot blot analysis of select mouse IgG1 fusion clones to verify the integrity of both the VHH and IgG1 Fc domains, exemplary clones are shown in FIG. 6B (II-B2 PMEPA-1 specific clone) and IG12 (non-specific clone) shown, with purified mouse IgG and purified VHH domain (clone ID5) as controls. Tissue culture supernatant (0.5 μL or 2.0 μL) spotted as indicated, and probed with HRP anti-mouse IgG or HRP anti-VHH. As shown in FIG. 6C, flow cytometric analysis of select human (upper panel, probed with PE-labeled anti-human IgG) and mouse (lower panel, probed with APC-labeled anti-mouse IgG) on PMEPA1-expressing 293F cultured cells.

A series of alternating magnetic-activated cell sorting (MACS)-based positive and negative selections were performed for nanobody generation. The MACS selections was followed by fluorescence-activating cell sorting (FACS)-based sorts. Synthetic biotinylated peptides representing the sequences of ectodomains of mouse and human PMEPA-1 were used as “anchors” for the selection. In some applications, control peptides comprised of scrambled sequences of amino acids (aa) are used. Nb-expressing yeast (the expression is determined by the inducible hemagglutinin (HA) tag in the construct) was used as a starting population. During a negative selection (or “pre-cleaning”), non-specific clones were selected for binding to the anti-biotin magnetic beads in the absence of a relevant peptide. Those clones were discarded, and the remaining clones were subjected to a positive selection using a PMEPA-1 peptide. Since some non-specific binding of HA− yeast occurs, further enrichment of HA+ clones bound to the peptide was performed by FACS-based sort (FIG. 7).

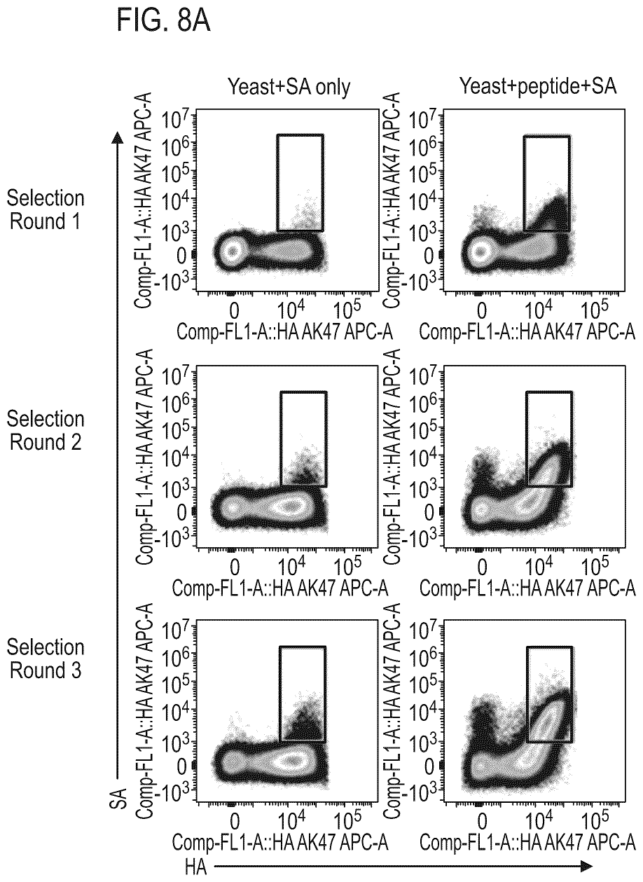

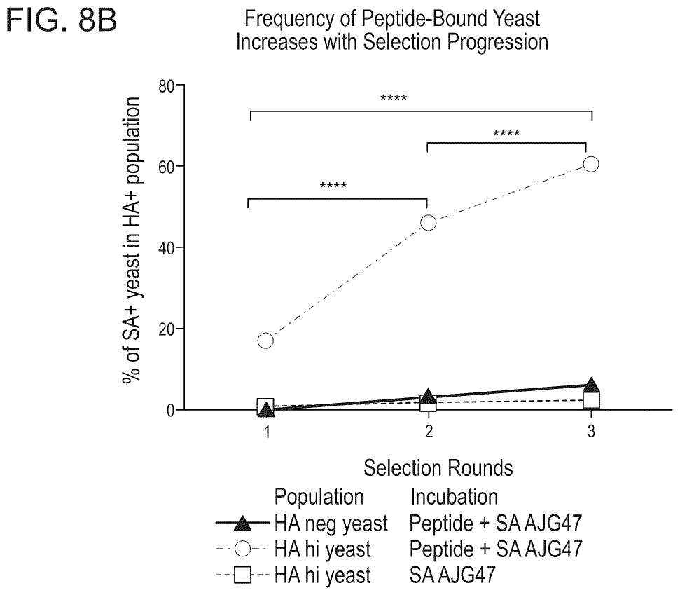

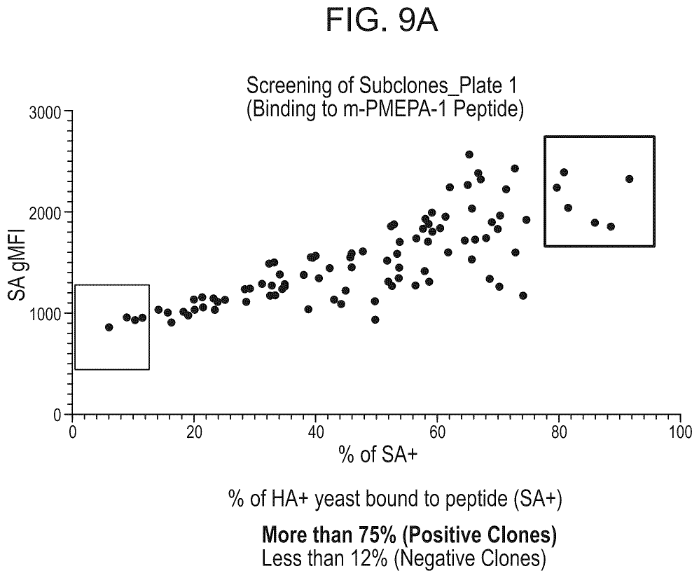

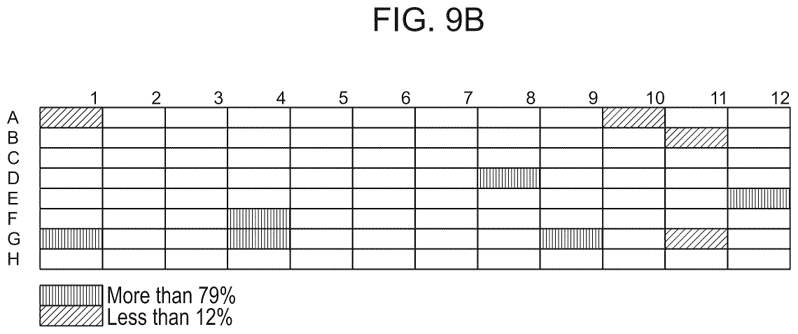

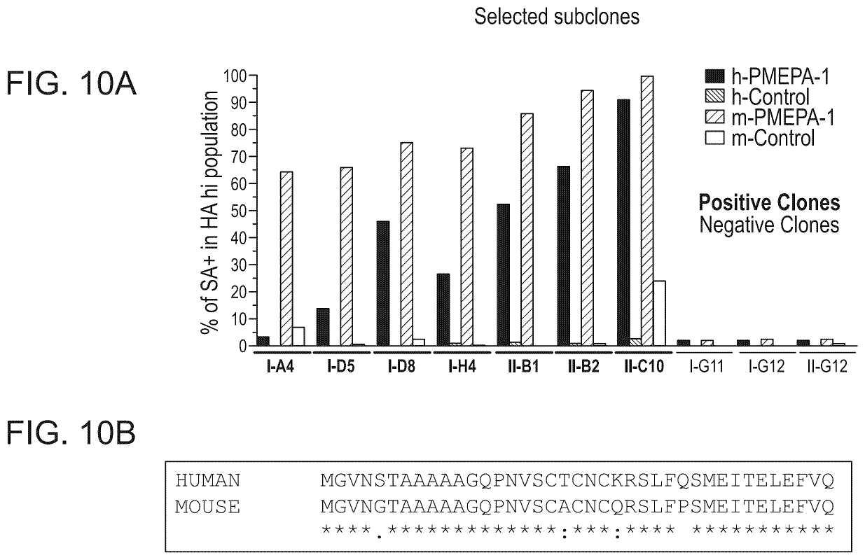

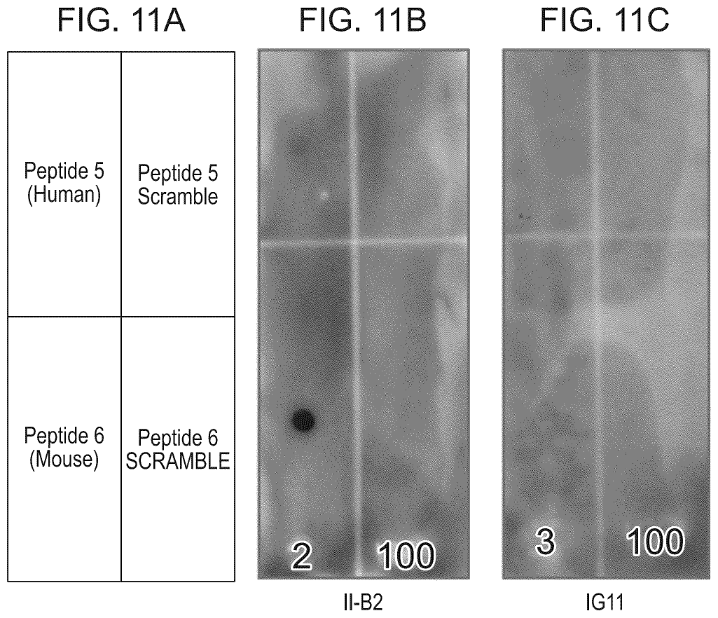

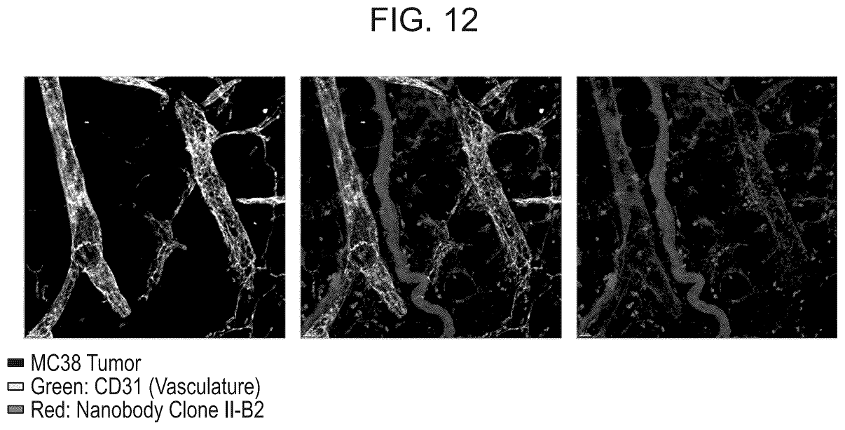

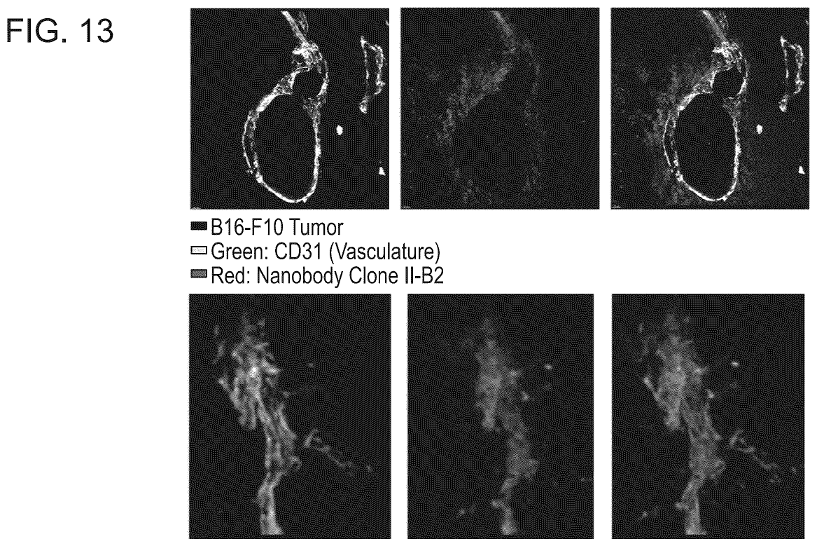

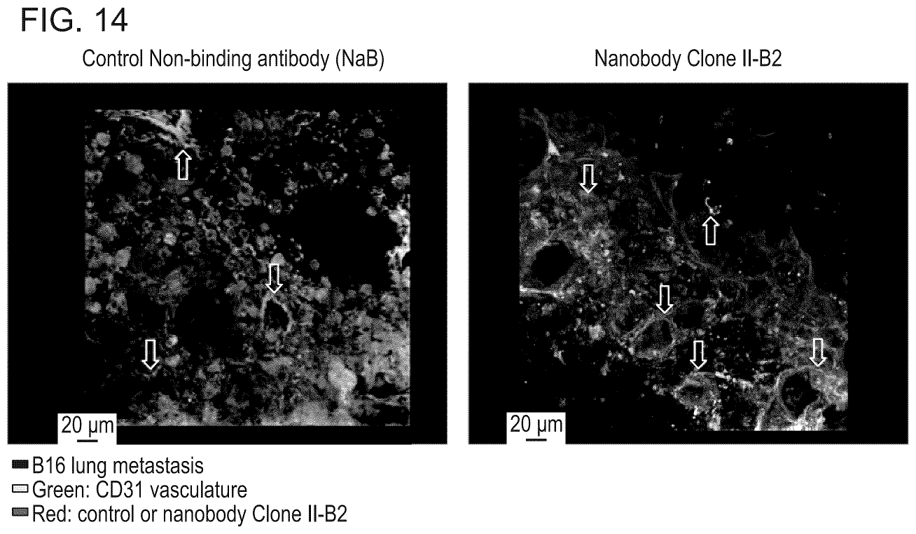

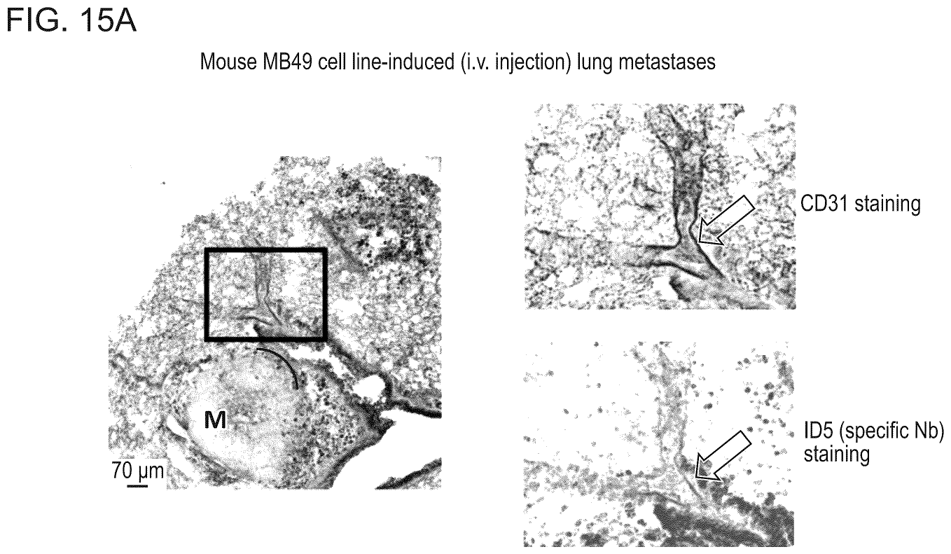

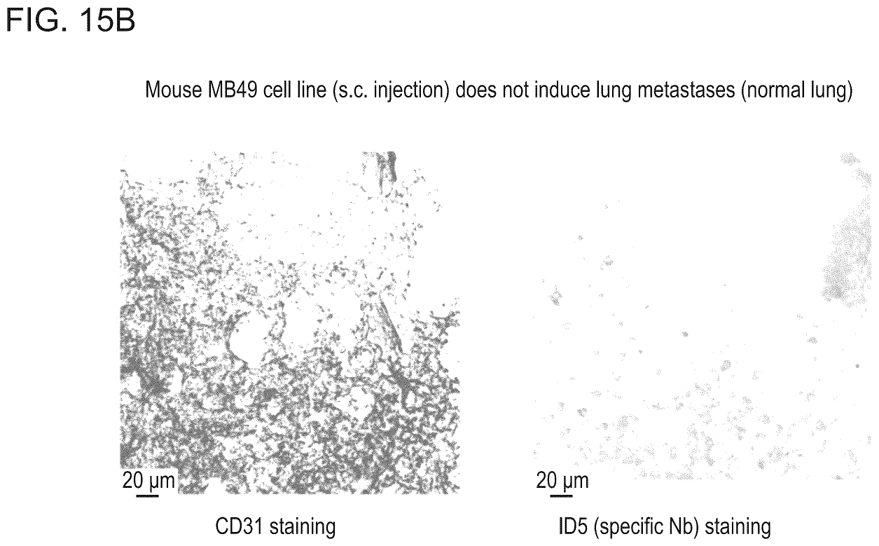

After each round of selection, the binding efficiency of the remaining HA+ clones to the specific peptide was assessed by FACS. The yeast was incubated with the biotinylated peptide, washed and stained with a streptavidin (SA)-conjugated fluorochrome. Peptide-bound yeast was recognized as a HA+SA+ population (FIG. 8A). With selection progression, the frequency of that population increased (FIG. 8B). After several rounds of MACS selection, followed by FACS sort of bulk HA+SA+ yeast cells, the remaining Nb-producing yeast clones were subcloned either by a serial dilution or by a single cell sort. After subclones were established and expanded, they were screened for those that exhibit both the strongest binding to the specific peptide and the weakest binding to the control peptide. Based on these parameters, hundreds of individual subclones were analyzed (FIG. 9), and the best positive and negative candidates were selected (FIG. 10A). Although all rounds of selection were done on mouse PMEPA-1 peptide (m-PMEPA-1), due to the high (79% aa, FIG. 10B) homology between mouse and human PMEPA-1 ectodomain, the majority of the selected subclones showed sufficient (although lower than to m-PMEPA-1) binding to human PMEPA-1 peptide (h-PMEPA-1). The selected subclones were subjected to further analysis. Seven unique nanobody species reactive with PMEPA-1 were isolated (Table 5), as well as 3 unique species with little or no reactivity to PMEPA-1 which served as negative control samples. Each identified subclone was transferred into an inducible bacterial expression vector for medium- to large-scale production, and preparations of each have been generated and purified. Retention of specificity for PMEPA-1 ECD has been verified by dot blot analysis (examples in FIGS. 11B and 11C), and a single subclone was tested for reactivity with tumor microvasculature in frozen microsections of both murine MC38 and B16-F10 primary tumors (FIGS. 12 and 13). This single subclone also demonstrated reactivity with tumor microvasculature in frozen microsections of mouse B16 lung metastases (FIG. 14). In addition, subclone ID-5 reacted with CD31+ endothelial cells in a vessel adjacent to MB49 lung metastastes but did not react in normal metastasis-free lung (FIGS. 15A and 15B). This data demonstrates that specificity for PMEPA-1 ECD is independent of tumor localization.

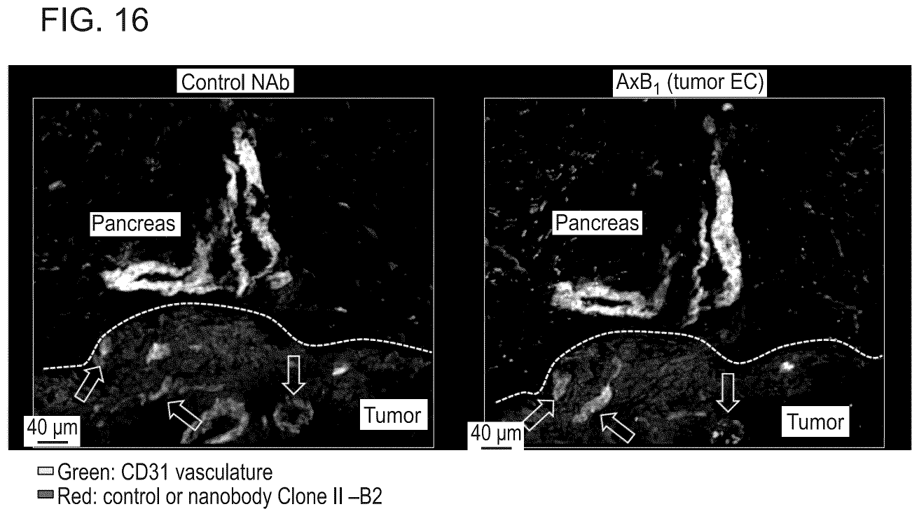

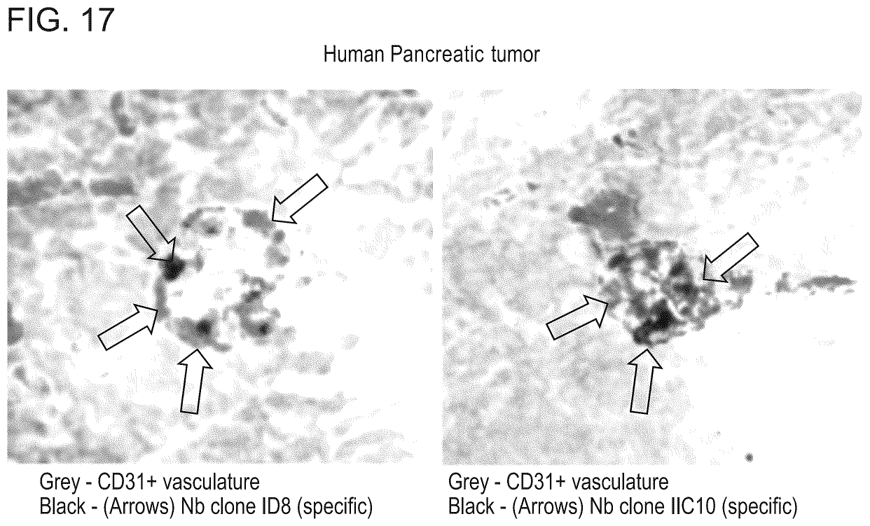

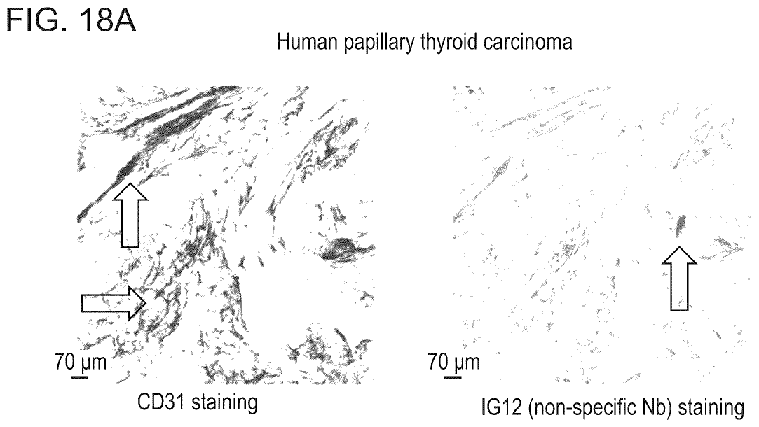

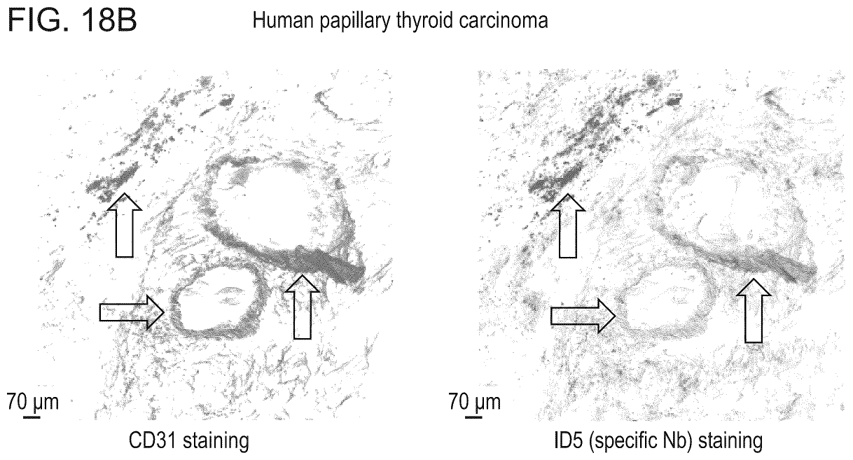

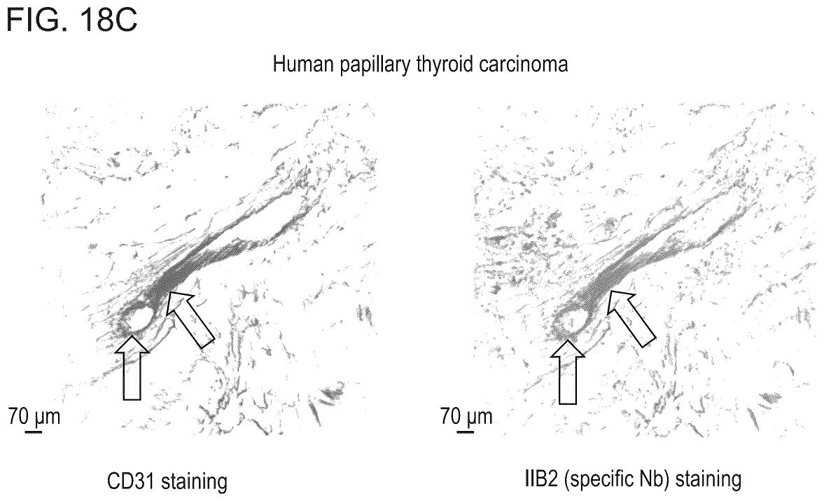

Furthermore, this subclone exhibited reactivity to frozen serial sections of human pancreatic cancer and surrounding non-malignant tissue (FIG. 16). Additional PMEPA 1-specific nanobody clones (ID8 and IIC10) were tested on frozen serial sections of human pancreatic cancer. FIG. 17 demonstrates that additional PMEPA-1 nanobodies react with the CD31+ vasculature endothelium in human pancreatic tumors. PMEPA-1 specific nanobodies (ID-5, II B-2) exhibit reactivity to frozen serial sections of human papillary thyroid carcinoma, while no reactivity was demonstrated with the nonspecific subclone IG-12 (FIGS. 18A-18C). This data demonstrates that the PMEPA-1 nanobodies specifically react with the vasculature endothelium in various human tumors.

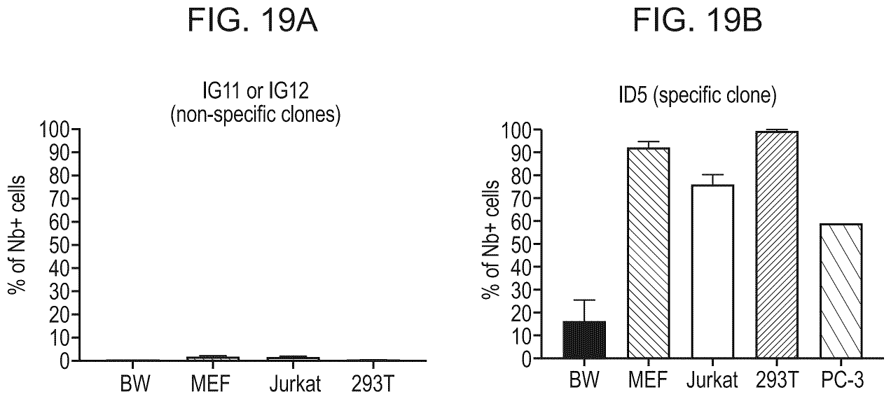

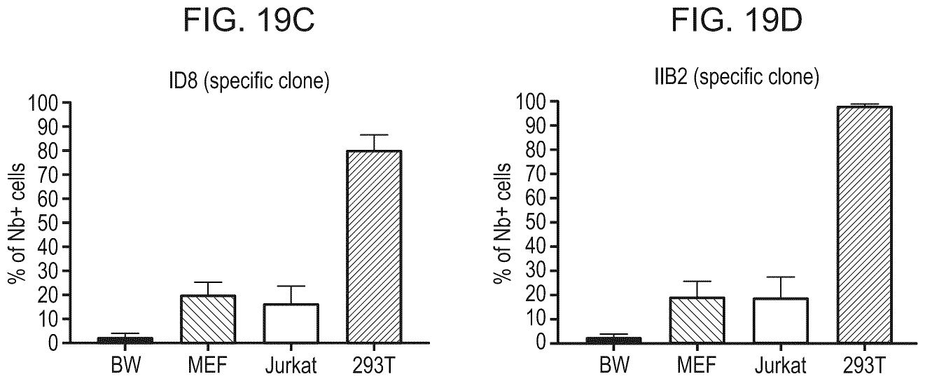

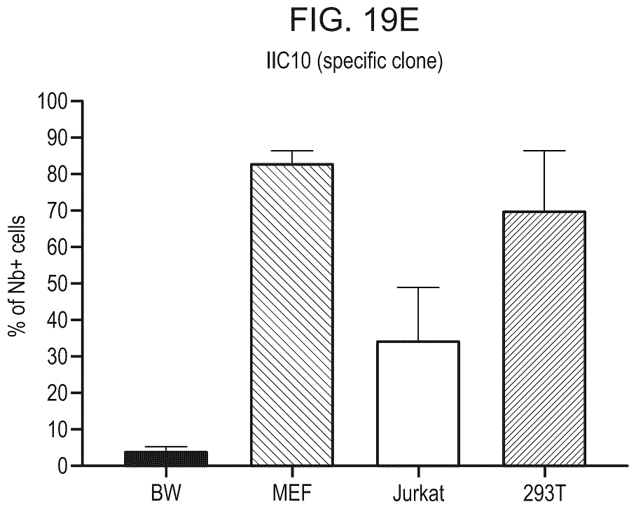

FIGS. 19A-19E demonstrates binding of specific and non-specific nanobody clones to various human and mouse cells lines by FACS. The mouse thymoma cell line BW demonstrated no mRNA PMEPA 1 expression by RT-PCR. However, the rest of the cell lines tested show PMEPA 1 expression on the mRNA level, and the cell lines represent human T cell leukemia (Jurkat), human embryonic kidney (23T), human prostate cancer (PC3) and mouse embryonic fibroblasts (MEF).

### Example 5: Demonstration of Tumor Vessel Targeting In Vivo Using Nanobodies Against PMEPA-1

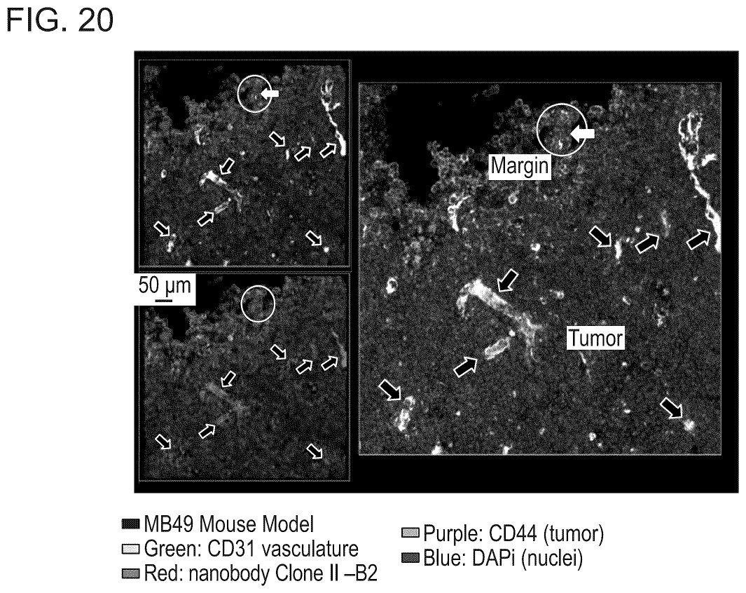

The single nanobody subclone described in Example 4 (nanobody clone II B-2) was intravenously injected into tumor bearing mice (MB49 model) on Day 12. Five minutes after IV administration, the tumors were harvested and subjected to immunohistochemistry as described in Example 4. As shown in FIG. 20, the PMEPA-1 nanobody accumulated in the tumor vessels in the mouse, but this nanobody did not accumulate in the vessels in the periphery. Thus, the PMEPA-1 nanobody specifically targeted the tumor vasculature in the living mouse.

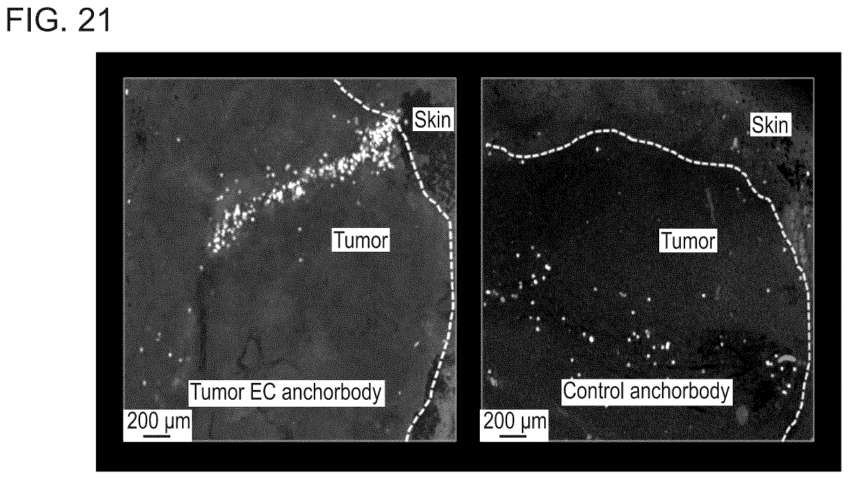

In addition, fluorescent microspheres were coated with PMEPA-1 nanobodies (nanobody clone II B-2) to investigate if PMEPA-1 specific nanobodies could be integrated with a therapeutic modality and retain its ability to target the tumor vasculature in vivo. The nanobody-coated microspheres (1 m) were intravenously injected into tumor bearing mice (MB49 model) on Day 12, and the tumors were harvested 5 minutes after administration. As shown in FIG. 21, the nanobody-coated fluorescent microspheres targeted the tumor microvessels in the living mouse.

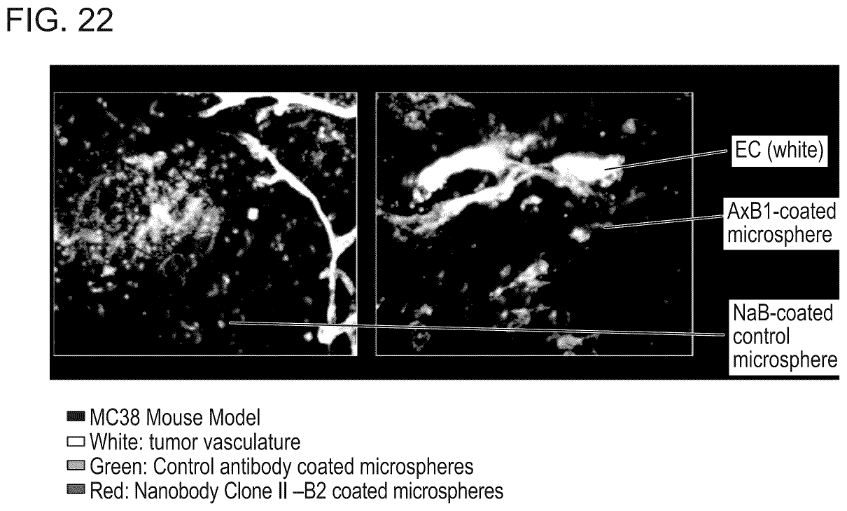

A mixture (1:1) of nanobody-coated microspheres (nanobody clone II B-2) and control microspheres were intravenously injected into a MC38 tumor bearing mouse. Tumors were harvested 5 minutes after administration, frozen sectioned and stained for CD31 to identify the endothelial cells. As shown in FIG. 22, the microspheres preferentially targeted the tumor when coated with the PMEPA-1 nanobody. Many microspheres localized to the extravascular space suggesting rapid trans-endothelial transport; suggesting the PMEPA-1 nanobodies may be used to translocate therapeutic payloads into the extravasculature space. This rapid extravasation suggests the transport might be via perivascular macrophages.

These data not only demonstrate the utility of the above gene lists in the identification of targetable tumor-specific genes, but also the ability to generate highly specific immunoreagents targeting these identified genes, such as PMEPA-1.

Tables 6-8 are exemplary nanobody amino acid sequences (Table 6), CDR sequences (Table 7), and nanobody nucleotide sequences (Table 8). Nanobodies IG11, IG12, and IIG12 were used a negative controls.

TABLE 6

| Clone | Nb Amino Acid Sequence |
|---|---|
| 1-A4 | QAGGSLRLSCAASGYIFSDTYMGWYRQAPGKEREFVAGINGGG |
| SEQ ID NO: 1 | TTNYADSVKGRFTISRDNAKNTVYLQMNSLKPEDTAVYYCAV |
| | YYGSFSWSLL |
| 1-D5 | AGGSLRLSCAASGNISYSYGMGWYRQAPGKEREFVAGITFGGS |
| SEQ ID NO: 2 | TYYADSVKGRFTISRDNAKNTVYLQMNSLKPEDTAVYYCAVY |
| | TRHVDTTFARHWYWGQGTQVTVSSLEHHH |
| I-D8 | AGGSLRLSCAASGNIFYGQPMGWYRQAPGKEREFVAGIGRGGS |
| SEQ ID NO: 3 | TYYADSVKGRFTISRDNAKNTVYLQMNSLKPEDTAVYYCAVL |
| | SQYPYRHTYWGQGTQVTVSSLEHH |
| I-H4 | QAGGSLRLSCAASGTISTYGMGWYRQAPGKEREFVAGIATGGT |
| SEQ ID NO: 4 | TYYADSVKGRFTISRDNAKNTVYLQMNSLKPEDTAVYYCAAL |
| | DKYARHYVYWGQGTQVTVSSLEHHHHHH |
| II-B1 | QAGGSLRLSCAASGTIFYRYSMGWYRQAPGKEREFVAGITEGS |
| SEQ ID NO: 5 | NTYYADSVKGRFTISRDNAKNTVYLQMNSLKPEDTAVYYCAV |
| | VQRVDLTYWGQGTQVTVSSLEHHHHHH |
| II-B2 | QAGGSLRLSCAASGNIFRVIGMGWYRQAPGKEREFVAGIGSGS |
| SEQ ID NO: 6 | STYYADSVKGRFTISRDNAKNTVYLQMNSLKPEDTAVYYCAAF |
| | TYPYYDQTKLLPYWGQGTQVTVSSLEHHHHHH |
| II-C10 | QAGGSLRLSCAASGTIFPRANMGWYRQAPGKEREFVAGITLGG |
| SEQ ID NO: 7 | TTYYADSVKGRFTISRDNAKNTVYLQMNSLKPEDTAVYYCAV |
| | VYKTYRYQEILYYYWGQGTQVTVSSLEHHHHHH |
| IG11 | QAGGSLRLSCAASGNIFRSSAMGWYRQAPGKEREFVAGITYGT |
| SEQ ID NO: 8 | NTYYADSVKGRFTISRDNAKNTVYLQMNSLKPEDTAVYYCAA |
| | STPARDALSQDLWYWGQGTQVTVSSLEHHHHHH |
| IG12 | QAGGSLRLSCAASGSIFPYSFMGWYRQAPGKEREFVAGISPGSN |
| SEQ ID NO: 9 | TYYADSVKGRFTISRDNAKNTVYLQMNSLKPEDTAVYYCAVV |
| | SDYQSHYYWGQGTQVTVSSLEHHHHHH |
| IIG12 | QAGGSLRLSCAASGTIFFRSYMGWYRQAPGKEREFVAGIGYGG |
| SEQ ID NO: 10 | NTYYADSVKGRFTISRDNAKNTVYLQMNSLKPEDTAVYYCAV |
| | YNRDYISPYGAHTYWGQGTQVTVSSLEHHHHHH |

TABLE 7

| | Amino Acid Sequence | | |
|---|---|---|---|
| Clone | CDR 1 | CDR2 | CDR3 |
| 1-D5 | NISYSYG | GITFGGS | VYTRHVDTTFARHW |
| | SEQ ID NO: 14 | SEQ ID NO: 15 | SEQ ID NO: 16 |
| I-D8 | NIFYGQP | GIGRGGS | VLSQYPYRHT |
| | SEQ ID NO: 17 | SEQ ID NO: 18 | SEQ ID NO: 19 |
| I-H4 | TISTYG | GIATGGT | ALDKYARHYV |
| | SEQ ID NO: 20 | SEQ ID NO: 21 | SEQ ID NO: 22 |
| II-B1 | TIFYRYS | GITEGSN | VVQRVDLT |
| | SEQ ID NO: 23 | SEQ ID NO: 24 | SEQ ID NO: 25 |
| II-B2 | NIFRVIG | GIGSGSS | AFTYPYYDQTKLLP |
| | SEQ ID NO: 26 | SEQ ID NO: 27 | SEQ ID NO: 28 |
| II-C10 | TIFPRAN | GITLGGT | VVYKTYRYQEILYY |
| | SEQ ID NO: 29 | SEQ ID NO: 30 | SEQ ID NO: 31 |
| IG11 | NIFRSSA | GITYGTN | ASTPARDALSQDLW |
| | SEQ ID NO: 32 | SEQ ID NO: 33 | SEQ ID NO: 34 |
| IG12 | SIFPYSF | GISPGSN | VVSDYQSHYY |
| | SEQ ID NO: 35 | SEQ ID NO: 36 | SEQ ID NO: 37 |
| IIG12 | TIFFRSY | GIGYGGN | VYNRDYISPYGAHT |
| | SEQ ID NO: 38 | SEQ ID NO: 39 | SEQ ID NO: 40 |

TABLE 8

| Clone | Nb Nucleotide Sequence |
|---|---|
| 1-D5 | GCGGGCGGCAGCCTGCGCCTGAGCTGCGCGGCGAGCGGCAA |
| SEQ ID NO: 42 | TATTTCTTACTCTTACGGTATGGGCTGGTATCGCCAGGCGCC |
| | GGGCAAAGAACGCGAATTTGTTGCCGGTATTACTTTCGGTGG |
| | TAGTACCTATTATGCGGATAGCGTGAAAGGCCGCTTTACCAT |
| | TAGCCGCGATAACGCGAAAAACACCGTGTATCTGCAGATGA |
| | ACAGCCTGAAACCGGAAGATACCGCGGTGTATTATTGCGCG |
| | GTTTACACTCGTCACGTTGACACTACTTTCGCTCGTCATTGGT |
| | ATTGGGGCCAGGGCACCCAGGTGACCGTGAGCAGCCTCGAG |
| | CACCACCACCACCACCAC |
| I-D8 | GCGGGCGGCAGCCTGCGCCTGAGCTGCGCGGCGAGCGGCAA |
| SEQ ID NO: 43 | TATTTTTTACGGTCAGCCGATGGGCTGGTATCGCCAGGCGCC |
| | GGGCAAAGAACGCGAATTTGTTGCCGGTATTGGTCGTGGTG |
| | GTAGTACCTATTATGCGGATAGCGTGAAAGGCCGCTTTACCA |
| | TTAGCCGCGATAACGCGAAAAACACCGTGTATCTGCAGATG |
| | AACAGCCTGAAACCGGAAGATACCGCGGTGTATTATTGCGC |
| | GGTTCTGTCTCAGTACCCGTACCGTCATACTTATTGGGGCCA |
| | GGGCACCCAGGTGACCGTGAGCAGCCTCGAGCACCACCACC |
| | ACCACCAC |
| I-H4 | GGCAGCCTGCGCCTGAGCTGCGCGGCGAGCGGCACTATTTCT |
| SEQ ID NO: 44 | ACTTACGGTATGGGCTGGTATCGCCAGGCGCCGGGCAAAGA |
| | ACGCGAATTTGTTGCCGGTATTGCTACTGGTGGTACTACCTA |
| | TTATGCGGATAGCGTGAAAGGCCGCTTTACCATTAGCCGCGA |
| | TAACGCGAAAAACACCGTGTATCTGCAGATGAACAGCCTGA |
| | AACCGGAAGATACCGCGGTGTATTATTGCGCGGCTCTGGAC |
| | AAATACGCTCGTCATTATGTTTATTGGGGCCAGGGCACCCAG |
| | GTGACCGTGAGCAGCCTCGAGCACCACCACCACCACCAC |
| II-B1 | GCGGGCGGCAGCCTGCGCCTGAGCTGCGCGGCGAGCGGCAC |
| SEQ ID NO: 45 | TATTTTTTACCGTTACTCTATGGGCTGGTATCGCCAGGCGCC |
| | GGGCAAAGAACGCGAATTTGTTGCCGGTATTACTGAAGGTA |
| | GTAATACCTATTATGCGGATAGCGTGAAAGGCCGCTTTACCA |
| | TTAGCCGCGATAACGCGAAAAACACCGTGTATCTGCAGATG |
| | AACAGCCTGAAACCGGAAGATACCGCGGTGTATTATTGCGC |
| | GGTTGTTCAGCGTGTTGACCTTACTTATTGGGGCCAGGGCAC |
| | CCAGGTGACCGTGAGCAGCCTCGAGCACCACCACCACCACC |
| | AC |
| II-B2 | GCGGGCGGCAGCCTGCGCCTGAGCTGCGCGGCGAGCGGCAA |
| SEQ ID NO: 46 | TATTTTTCGTGTTATCGGTATGGGCTGGTATCGCCAGGCGCC |
| | GGGCAAAGAACGCGAATTTGTTGCCGGTATTGGTTCTGGTAG |
| | TAGTACCTATTATGCGGATAGCGTGAAAGGCCGCTTTACCAT |
| | TAGCCGCGATAACGCGAAAAACACCGTGTATCTGCAGATGA |
| | ACAGCCTGAAACCGGAAGATACCGCGGTGTATTATTGCGCG |
| | GCTTTCACTTACCCGTACTACGACCAGACTAAACTGCTTCCG |
| | TATTGGGGCCAGGGCACCCAGGTGACCGTGAGCAGCCTCGA |
| | GCACCACCACCACCACCAC |
| II-C10 | GCGGGCGGCAGCCTGCGCCTGAGCTGCGCGGCGAGCGGCAC |
| SEQ ID NO: 47 | TATTTTTCCGCGTGCTAACATGGGCTGGTATCGCCAGGCGCC |
| | GGGCAAAGAACGCGAATTTGTTGCCGGTATTACTCTGGGTGG |
| | TACTACCTATTATGCGGATAGCGTGAAAGGCCGCTTTACCAT |
| | TAGCCGCGATAACGCGAAAAACACCGTGTATCTGCAGATGA |
| | ACAGCCTGAAACCGGAAGATACCGCGGTGTATTATTGCGCG |
| | GTTGTTTACAAAACTTACCGTTACCAGGAAATCCTGTATTAC |
| | TATTGGGGCCAGGGCACCCAGGTGACCGTGAGCAGCCTCGA |
| | GCACCACCACCACCACCAC |
| IG11 | GCGGGCGGCAGCCTGCGCCTGAGCTGCGCGGCGAGCGGCAA |
| SEQ ID NO: 48 | TATTTTTCGTTCTTCTGCTATGGGCTGGTATCGCCAGGCGCCG |
| | GGCAAAGAACGCGAATTTGTTGCCGGTATTACTTACGGTACT |
| | AATACCTATTATGCGGATAGCGTGAAAGGCCGCTTTACCATT |
| | AGCCGCGATAACGCGAAAAACACCGTGTATCTGCAGATGAA |
| | CAGCCTGAAACCGGAAGATACCGCGGTGTATTATTGCGCGG |
| | CTTCTACTCCGGCTCGTGACGCTCTGTCTCAGGACCTTTGGTA |
| | TTGGGGCCAGGGCACCCAGGTGACCGTGAGCAGCCTCGAGC |
| | ACCACCACCACCACCAC |
| IG12 | GCGGCGAGCGGCTCTATTTTTCCGTACTCTTTCATGGGCTGG |
| SEQ ID NO: 49 | TATCGCCAGGCGCCGGGCAAAGAACGCGAATTTGTTGCCGG |
| | TATTAGTCCGGGTAGTAATACCTATTATGCGGATAGCGTGAA |
| | AGGCCGCTTTACCATTAGCCGCGATAACGCGAAAAACACCG |
| | TGTATCTGCAGATGAACAGCCTGAAACCGGAAGATACCGCG |
| | GTGTATTATTGCGCGGTTGTTTCTGACTACCAGTCTCATTACT |
| | ATTGGGGCCAGGGCACCCAGGTGACCGTGAGCAGCCTCGAG |
| | CACCACCACCACCACCAC |
| IIG12 | GCGGGCGGCAGCCTGCGCCTGAGCTGCGCGGCGAGCGGCAC |
| SEQ ID NO: 50 | TATTTTTTTCCGTTCTTACATGGGCTGGTATCGCCAGGCGCCG |
| | GGCAAAGAACGCGAATTTGTTGCCGGTATTGGTTACGGTGGT |
| | AATACCTATTATGCGGATAGCGTGAAAGGCCGCTTTACCATT |
| | AGCCGCGATAACGCGAAAAACACCGTGTATCTGCAGATGAA |
| | CAGCCTGAAACCGGAAGATACCGCGGTGTATTATTGCGCGG |
| | TTTACAACCGTGACTACATCTCTCCGTACGGTGCTCATACTT |
| | ATTGGGGCCAGGGCACCCAGGTGACCGTGAGCAGCCTCGAG |
| | CACCACCACCACCACCAC |

### REFERENCES

(1) Folkmnan, J. Tumor angiogenesis: therapeutic implications. N Engl J Med, 285, 1182-1186, (1971).
(2) Peske J D, Woods A B, Engelhard V H. Control of CD8 T-Cell Infiltration into Tumors by Vasculature and Microenvironment. Adv Cancer Res., 128, 263-307, (2015).
(3) Potente, M., Mdkinen, T. Vascular heterogeneity and specialization in development and disease. Nat Rev Mol Cell Biol 18, 477-494 (2017).
(4) Halhn, C., J. Rodrigo Mora, C. Sumen, and U. H. von Andrian. In vivo imaging of lymphocyte trafficking. Annu Rev Cell Dev Biol, 21, 581-603, (2005).
(5) Ley, K., and P. Gaehtgens. Endothelial, not hemodynamic, differences are responsible for preferential leukocyte rolling in rat mesenteric venules. Circ Res, 69, 1034-1041, (1991).
(6) Lawson, N. D., and B. M. Weinstein. Arteries and veins: making a difference with zebrafish. Nat Rev Genet, 3, 674-682, (2002).
(7) von Andrian, U. H., and C. R. Mackay. 2000. T-cell function and migration. Two sides of the same coin. N. Engl. J. Med., 343, 1020-1034, (2000).
(8) Rocha, S. F., and R. H. Adams. Molecular differentiation and specialization of vascular beds. Angiogenesis, 12, 139-147, (2009).
(9) Oliver, G., and R. S. Srinivasan. Endothelial cell plasticity: how to become and remain a lymphatic endothelial cell. Development, 137, 363-372, (2010).
(10) Thiriot, A. et al. Differential DARC/ACKR1 expression distinguishes venular from non-venular endothelial cells in murine tissues. BMC Biology, 15, 45, (2017).
(11) Ngiow, S. F. et al. A Threshold Level of Intratumor CD8+ T-cell PD1 Expression Dictates Therapeutic Response to Anti-PD1. Cancer Research, 75, 3800-3811, (2015).
(12) Vikram R. Juneja, Kathleen A. McGuire, Robert T. Manguso, Martin W. LaFleur, Natalie Collins, W. Nicholas Haining, Gordon J. Freeman, Arlene H. Sharpe. PD-L1 on tumor cells is sufficient for immune evasion in immunogenic tumors and inhibits CD8 T cell cytotoxicity. J Exp Med, 214, 895-904, (2014).
(13) Weninger, W., M. A. Crowley, N. Manjunath, and U. H. von Andrian. Migratory properties of naive, effector, and memory CD8(+) T cells. J. Exp. Med. 194, 953-966, (2001).
(14) Gerlach, C. et. al. The Chemokine Receptor CX3CR1 Defines Three Antigen-Experienced CD8 T Cell Subsets with Distinct Roles in Immune Surveillance and Homeostasis. Immunity, 45, 1270-1284, (2016).
(15) Gierahn, T. M. et al. Seq-Well: portable, low-cost RNA sequencing of single cells at high throughput. Nature Methods 14, 395-398, (2017).
(16) Wolf, K. et. al. Molecular identity of arteries, veins, and lymphatics. J. Vasc. Surgery., 69, 253-262, (2019).
(17) Nathaniel G. de la Paz and Patricia A. D'Amore. Arterial versus venous endothelial cells. Cell tissue, 335, 5-16, (2014).
(18) Zhao, Q. et. al. Single-Cell Transcriptome Analyses Reveal Endothelial Cell Heterogeneity in Tumors and Changes following Antiangiogenic Treatment. Cancer Research, 78, 2370-2383, (2018).
(19) Iso, T. et. al. Capillary Endothelial Fatty Acid Binding Proteins 4 and 5 Play a Critical Role in Fatty Acid Uptake in Heart and Skeletal Muscle. Arteriosclerosis, Thrombosis, and Vascular Biology, 33, 2549-2557, (2013).
(20) Rousseeuw, P. J. Silhouettes: A graphical aid to the interpretation and validation of cluster analysis. Journal of Computational and Applied Mathematics, 20, 53-65, (1987).
(21) Dong, X. et. al. Crystal Structure of the V Domain of Human Nectin-like Molecule-1/Syncam3/Tslll/Igsf4b, a Neural Tissue-specific Immunoglobulin-like Cell-Cell Adhesion Molecule. JBC, 281, 10610-10617, (2006).
(22) Wang X, Abraham S, McKenzie J A, Jeffs N, Swire M, Tripathi V B, Luhmann U F, Lange C A, Zhai Z, Arthur H M, Bainbridge J W, Moss S E, Greenwood J. LRG1 promotes angiogenesis by modulating endothelial TGF-β signalling. Nature, 499, 306-11, (2013).
(23) Hanzelmann, S., Castelo, R. & Guinney, J. GSVA: gene set variation analysis for microarray and RNA-Seq data. BMC Bioinformatics, 14, 7, (2013).
(24) Kalucka, J. et. al. Single-Cell Transcriptome Atlas of Murine Endothelial Cells. Cell, 180, 764-779, (2020).
(25) Muz, B., de la Puente, P., Azab, F., & Azab, A. K. The role of hypoxia in cancer progression, angiogenesis, metastasis, and resistance to therapy. Hypoxia (Auckland, N.Z.), 3, 83-92, (2015).
(26) Tran-Dinh et. al., HDL and endothelial protection. BJP, 169, 493-511, (2013).
(27) Narimasa Miho, Takafumi Ishida, Noriko Kuwaba, Mari Ishida, Keiko Shimote-Abe, Kumiko Tabuchi, Tetsuya Oshima, Masao Yoshizumi, Kazuaki Chayama. Role of the JNK pathway in thrombin-induced ICAM-1 expression in endothelial cells. Cardiovascular Research, 68, 289-298, (2005).
(28) Saul, L., Ilieva, K. M., Bax, H. J., Karagiannis, P., Correa, I., Rodriguez-Hernandez, I., Josephs, D. H., Tosi, I., Egbuniwe, I. U., Lombardi, S., Crescioli, S., Hobbs, C., Villanova, F., Cheung, A., Geh, J. L., Healy, C., Harries, M., Sanz-Moreno, V., Fear, D. J., Spicer, J. F., . . . Karagiannis, S. N. IgG subclass switching and clonal expansion in cutaneous melanoma and normal skin. Scientific reports, 6, 29736, (2016).
(29) Prabir Kumar Chakraborty and Soumyajit Banerjee Mustafi. Heat Shock Proteins: Heating up skin cancer biology. Journal of Dermatitis, 1, 1-4, (2015).
(30) Muchemwa, Faith Chengetayi; Nakatsura, Tetsuya; Fukushima, Satoshi; Nishimura, Yasuharu; Kageshita, Toshiro; Ihn, Hironobu. Differential expression of heat shock protein 105 in melanoma and melanocytic naevi. Melanoma Research, 18, 166-171, (2008).
(31) Pleunie van den Borne, Paul H. A. Quax, Imo E. Hoefer, and Gerard Pasterkamp. The Multifaceted Functions of CXCL10 in Cardiovascular Disease. Biomed Research International, 2014, 893106, (2014).
(32) Gabriele S. V. Campanella, Jan Grimm, Lindsay A. Manice, Richard A. Colvin, Benjamin D. Medoff, Gregory R. Wojtkiewicz, Ralph Weissleder, Andrew D. Luster. Oligomerization of CXCL10 Is Necessary for Endothelial Cell Presentation and In Vivo Activity. The Journal of Immunology, 177, 6991-6998, (2006).
(33) Ji, P., Diederichs, S., Wang, W. et al. MALAT-1, a novel noncoding RNA, and thymosin β4 predict metastasis and survival in early-stage non-small cell lung cancer. Oncogene 22, 8031-8041 (2003).
(34) Xue, Aiqun MSc*; Scarlett, Christopher J. PhD*; Jackson, Christopher J. PhDt; Allen, Barry J. DSc, FAPS‡; Smith, Ross C. M D, FRACS* Prognostic Significance of Growth Factors and the Urokinase-Type Plasminogen Activator System in Pancreatic Ductal Adenocarcinoma. Pancreas, 36, 160-167, (2008).
(35) Chen, E. Y., Tan, C. M., Kou, Y. et al. Enrichr: interactive and collaborative HTML5 gene list enrichment analysis tool. BMC Bioinformatics 14, 128 (2013).
(36) Kuleshov, M. V., Jones, M. R., Rouillard, A. D., Fernandez, N. F., Duan, Q., Wang, Z., Koplev, S., Jenkins, S. L., Jagodnik, K. M., Lachmann, A., McDermott, M. G., Monteiro, C. D., Gundersen, G. W., & Ma'ayan, A. Enrichr: a comprehensive gene set enrichment analysis web server 2016 update. Nucleic acids research, 44(W1), W90-W97, (2016).
(37) Dunham, I., Kundaje, A., Aldred, S. et al. An integrated encyclopedia of DNA elements in the human genome. Nature 489, 57-74 (2012).
(38) Davis, C. A., Hitz, B. C., Sloan, C. A., Chan, E. T., Davidson, J. M., Gabdank, I., Hilton, J. A., Jain, K., Baymuradov, U. K., Narayanan, A. K., Onate, K. C., Graham, K., Miyasato, S. R., Dreszer, T. R., Strattan, J. S., Jolanki, O., Tanaka, F. Y., & Cherry, J. M. The Encyclopedia of DNA elements (ENCODE): data portal update. Nucleic acids research, 46(D1), D794-D801, (2018).
(39) Mogensen Trine H. IRF and STAT Transcription Factors—From Basic Biology to Roles in Infection, Protective Immunity, and Primary Immunodeficiencies. Frontiers in Immunology, 9, 3047, (2019).
(40) Min, Y., Ghose, S., Boelte, K. et al. C/EBP-δ regulates VEGF-C autocrine signaling in lymphangiogenesis and metastasis of lung cancer through HIF-1α. Oncogene 30, 4901-4909 (2011).
(41) Wen, Y., Zhou, X., Lu, M. et al. Bclaf1 promotes angiogenesis by regulating HIF-1α transcription in hepatocellular carcinoma. Oncogene 38, 1845-1859 (2019).
(42) Song, H., Suehiro, J., Kanki, Y., Kawai, Y., Inoue, K., Daida, H., Yano, K., Ohhashi, T., Oettgen, P., Aird, W. C., Kodama, T., & Minami, T. Critical role for GATA3 in mediating Tie2 expression and function in large vessel endothelial cells. The Journal of biological chemistry, 284, 29109-29124, (2009).
(43) Fu, M. et. al. Egr-1 target genes in human endothelial cells identified by microarray analysis. Gene, 315, 33-41, (2003).
(44) Vicari, A. P. et. al. TECK: A Novel C C Chemokine Specifically Expressed by Thymic Dendritic Cells and Potentially Involved in T Cell Development. Immunity, 7, 291-301, (1997).
(45) Luster, A. D. et. al. Molecular and biochemical characterization of a novel y-interferon-inducible protein. JBC, 263, 12036-12043, (1988).
(46) Zhang, H. et. al. mRNA-Binding Protein ZFP36 Is Expressed in Atherosclerotic Lesions and Reduces Inflammation in Aortic Endothelial Cells. Arteriosclerosis, Thrombosis, and Vascular Biology, 33, 1212-1220, (2013).
(47) Cohnheim J. Lectures on General Pathology: A Handbook for Practitioners and Students. London: The New Sydenham Society; 1889.
(48) Yano K, Gale D, Massberg S, Cheruvu P K, Monahan-Earley R, Morgan E S, Haig D, von Andrian U H, Dvorak A M, Aird W C. Phenotypic heterogeneity is an evolutionarily conserved feature of the endothelium. Blood, 109, 613-5, (2007).
(49) Atherton A, Born G V R. Quantitative investigation of the adhesiveness of circulating polymorphonuclear leucocytes to blood vessel walls. J Physiol., 222, 447-74, (1972).
(50) von Andrian U H, Chambers J D, McEvoy L M, Bargatze R F, Arfors K E, Butcher E C. Two-step model of leukocyte-endothelial cell interaction in inflammation: distinct roles for LECAM-1 and the leukocyte beta 2 integrins in vivo. Proc Natl Acad Sci USA, 88, 7538-42, (1991).
(51) T. M. Gierahn, M. H. 2. Wadsworth, T. K. Hughes, B. D. Bryson, A. Butler, R. Satija, S. Fortune, J. C. Love and A. K. Shalek. Seq-Well: portable, low-cost RNA sequencing of single cells at high throughput,” Nat Methods, pp. 395-398, 13 Apr. 2017.
(52) A. Dobin and e. al., “STAR: ultrafast universal RNA-seq aligner,” Bioinformatics, no. 29, pp. 15-21, 2013.
(53) E. Z. Macosko, “Highly parallel genome-wide expression profiling of individual cells using nanoliter droplets.,” Cell, no. 161, pp. 1202-1214, 2015.
(54) R. Satija, J. A. Farrell, D. Gennert, A. F. Schier and A. Regev, “Spatial reconstruction of single-cell gene expression data,” Nat. Biotechnol., no. 33, pp. 495-502, 2015.
(55) F. A. Wolf, P. Angerer and F. J. Theis, “SCANPY: large-scale single-cell gene expression data analysis.,” Genome Biol., no. 19, p. 15, 2018.
(56) E. e. a. Becht, “Dimensionality reduction for visualizing single-cell data using UMAP.,” Nat. Biotechnol., no. 37, pp. 38-44, 2018.
(57) C. S. McGinnis, L. M. Murrow and Z. J. Gartner, “DoubletFinder: Doublet Detection in Single-Cell RNA Sequencing Data Using Artificial Nearest Neighbors,” Cell Systems, vol. 8, no. 4, pp. 329-337, 2019.
(58) Stuart, T., Butler, A. et. al. Comprehensive Integration of Single-Cell Data. Cell, 177, 1888-1902, (2019).
(59) Rousseeuw, P. J. Silhouettes: A graphical aid to the interpretation and validation of cluster analysis. Journal of Computational and Applied Mathematics, 20, 53-65, (1987).
(60) Hanzelmann, S., Castelo, R. & Guinney, J. GSVA: gene set variation analysis for microarray and RNA-Seq data. BMC Bioinformatics, 14, 7, (2013).
(61) Kalucka, J. et. al. Single-Cell Transcriptome Atlas of Murine Endothelial Cells. Cell, 180, 764-779, (2020).
(62) Smyth G K. Linear models and empirical bayes methods for assessing differential expression in microarray experiments. Stat Appl Genet Mol Biol. 3, Article3, (2004).
(63) Chen, E. Y., Tan, C. M., Kou, Y. et al. Enrichr: interactive and collaborative HTML5 gene list enrichment analysis tool. BMC Bioinformatics 14, 128 (2013).
(64) Kuleshov, M. V., Jones, M. R., Rouillard, A. D., Fernandez, N. F., Duan, Q., Wang, Z., Koplev, S., Jenkins, S. L., Jagodnik, K. M., Lachmann, A., McDermott, M. G., Monteiro, C. D., Gundersen, G. W., & Ma'ayan, A. Enrichr: a comprehensive gene set enrichment analysis web server 2016 update. Nucleic acids research, 44(W1), W90-W97, (2016).
(65) Siegel, R. L., K. D. Miller, and A. Jemal. 2016. Cancer statistics, 2016. C A Cancer J Clin 66: 7-30.
(66) Pauken, K. E., and E. J. Wherry. 2015. Overcoming T cell exhaustion in infection and cancer. Trends Immunol 36: 265-276.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. An antibody or antigen binding fragment thereof, which specifically binds to Prostate Transmembrane Protein, Androgen Induced 1 (PMEPA-1).

2. The antibody or antigen binding fragment thereof of claim 1 selected from the group consisting of a monoclonal antibody, human antibody, a humanized antibody, a chimeric antibody, a recombinant antibody, a multispecific antibody, or an antigen-binding fragment thereof; wherein the antigen-binding fragment is 1) an Fv, Fab, F(ab′)2, Fab′, dsFv, scFv, or sc(Fv)2; or 2) a diabody, ScFv, SMIP, single chain antibody, affibody, avimer, or nanobody; or 3) a single domain antibody, and an antigen binding fragment of any of the foregoing.

3. (canceled)

4. The antibody or antigen binding fragment thereof of claim 1, wherein the antibody or antigen binding fragment thereof comprises:
(1) a CDR1 sequence comprising the amino acid sequence of SEQ ID NO: 14, a CDR2 sequence comprising the amino acid sequence of SEQ ID NO: 15, and a CDR3 sequence comprising the amino acid sequence of SEQ ID NO: 16; or
(2) a CDR1 sequence comprising the amino acid sequence of SEQ ID NO: 17, a CDR2 sequence comprising the amino acid sequence of SEQ ID NO: 18, and a CDR3 sequence comprising the amino acid sequence of SEQ ID NO: 19; or
(3) a CDR1 sequence comprising the amino acid sequence of SEQ ID NO: 20, a CDR2 sequence comprising the amino acid sequence of SEQ ID NO: 21, and a CDR3 sequence comprising the amino acid sequence of SEQ ID NO: 22; or
(4) a CDR1 sequence comprising the amino acid sequence of SEQ ID NO: 23, a CDR2 sequence comprising the amino acid sequence of SEQ ID NO: 24, and a CDR3 sequence comprising the amino acid sequence of SEQ ID NO: 25; or
(5) a CDR1 sequence comprising the amino acid sequence of SEQ ID NO: 26, a CDR2 sequence comprising the amino acid sequence of SEQ ID NO: 27, and a CDR3 sequence comprising the amino acid sequence of SEQ ID NO: 28; or
(6) a CDR1 sequence comprising the amino acid sequence of SEQ ID NO: 29, a CDR2 sequence comprising the amino acid sequence of SEQ ID NO: 30, and a CDR3 sequence comprising the amino acid sequence of SEQ ID NO: 31; or
(7) an amino acid sequence that has at least 90%, 91%, 92%, 93%, 94%, 95%, 96%, 97%, 98%, or 99% identity to, comprises, or consists of an amino acid sequence selected from SEQ ID NOs: 1-7; or
(8) the amino acid sequence of SEQ ID NO: 5 or SEQ ID NO: 6.

5.-7. (canceled)

8. A composition comprising 1) the antibody or antigen binding fragment thereof of claim 1, and 2) an agent that (a) induces cell death, or (b) induces an inflammatory response to a tumor vascular endothelial cell in which the expression of PMEPA-1 is upregulated as compared to expression in a non-tumor vascular endothelial cell control.

9. The composition of claim 8, wherein:
(a) the cell death is induced in a tumor vascular endothelial cell in which the expression of PMEPA-1 is upregulated as compared to expression in a non-tumor vascular endothelial cell control, and/or in a tumor cell;
(b) the agent that induces cell death is an agent that induces immunogenic cell death or an agent that induces non-immunogenic cell death;
(c) the agent is selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof;
(d) the agent that induces an inflammatory response is a TLR4 agonist or GP-130 agonist;
(e) the agent that induces cell death is a chemotherapeutic agent;
(f) the agent that induces cell death is an engineered CAR-immune cell, optionally the CAR-immune cell is a CAR-T cell, CAR-macrophages, CAR-monocyte, CAR-granulocyte, CAR-NK cell, or a CAR-NKT cell, or a tumor infiltrating lymphocyte (TL), or a cell expressing an antigen recognizing a tumor antigen or a cell expressing a receptor recognizing an antibody bound to the surface of a tumor cell; and/or
(g) the agent that induces cell death or the agent that induces an inflammatory response is coupled to or is co-administered with the antibody or antigen binding fragment thereof.

10.-15. (canceled)

16. The composition of claim 9, wherein the engineered CAR-T cell comprises a nucleic acid molecule comprising a nucleotide sequence having at least 90%, 91%, 92%, 93%, 94%, 95%, 96%, 97%, 98%, 99% sequence identity to SEQ ID NOs: 42-47.

17. (canceled)

18. A pharmaceutical composition comprising 1) the antibody or antigen binding fragment thereof of claim 1, and 2) a pharmaceutically acceptable carrier.

19. The pharmaceutical composition of claim 18, wherein the pharmaceutical composition comprises a lipid formulation, wherein the lipid formulation comprises a lipid nanoparticle.

20. (canceled)

21. A method of treating cancer in a subject in need thereof,
(1) wherein the cancer is characterized by a tumor vascular endothelial cell in which the expression PMEPA-1 is upregulated, comprising administering to the subject a composition comprising an antibody or antigen binding fragment thereof which binds to PMEPA-1 on the tumor vascular endothelial cell and an agent that induces cell death or an agent that induces an inflammatory response, optionally wherein the composition is the composition of claim 8; or
(2) comprising administering to the subject the composition of claim 8.

22. (canceled)

23. The method of claim 21, wherein the expression of the PMEPA-1 is upregulated as compared to a control level, wherein the control level is the level of expression of PMEPA-1 in a non-tumor vascular endothelial control cell.

24. (canceled)

25. The method of claim 21,
(a) further comprising identifying in the subject the presence of the tumor vascular endothelial cell in which the expression of PMEPA-1 is upregulated as compared to a non-tumor vascular endothelial control cell; and/or
(b) wherein the method elicits or enhances an immune response to the cancer, wherein the method increases the level or activity of intra-tumoral T cells, wherein the level or activity of intra-tumoral T cells are increased at least 1.5-fold, at least 2-fold, at least 2.5-fold, at least 3-fold, at least 3.5-fold, at least 4-fold, at least 4.5-fold, or at least 5-fold more after administration as compared to the level or activity of intra-tumoral T cells prior to administration.

26.-28. (canceled)

29. A method of treating cancer in a subject in need thereof, comprising:
(a) administering to a subject having cancer an immune effector cell expressing a chimeric antigen receptor (CAR), wherein the CAR comprises the antibody or antigen binding fragment thereof of claim 1, wherein the antibody or antigen binding fragment thereof binds to PMEPA-1 on a tumor vascular endothelial cell in which expression of PMEPA-1 is upregulated; or
(b) administering to a subject having cancer an immune effector cell expressing a chimeric antigen receptor (CAR), wherein the antibody or antigen binding fragment thereof of claim 1 is expressed on the cell surface of the immune effector cell, wherein the antibody or antigen binding fragment thereof binds to PMEPA-1 on a tumor vascular endothelial cell in which expression of PMEPA-1 is upregulated.

30. (canceled)

31. The method of claim 29, wherein;
(1) the immune effector cell is a T cell, macrophage, monocyte, granulocyte, natural killer (NK) cell, or natural killer T (NKT);
(2) further comprising identifying in the subject the presence of the tumor vascular endothelial cell in which the expression of PMEPA-1 is upregulated as compared to expression in a non-tumor vascular endothelial control cell; and/or
(3) the method elicits or enhances an immune response to the cancer, optionally by increasing the level or activity of intra-tumoral T cells, wherein the level or activity of intra-tumoral T cells is increased at least 1.5-fold, at least 2-fold, at least 2.5-fold, at least 3-fold, at least 3.5-fold, at least 4-fold, at least 4.5-fold, or at least 5-fold more after administration as compared to the level or activity of intratumoral T cells to prior administration.

32.-35. (canceled)

36. A method of diagnosing or prognosing cancer in a subject, comprising determining the expression of PMEPA-1 on a tumor vascular endothelial cell, wherein upregulation of expression of PMEPA-1 on the tumor vascular endothelial cell as compared to a control level is indicative of the presence or progression of the cancer, optionally, wherein the control level is the level in a non-tumor endothelial control cell, and, optionally, wherein the method further comprises a step of administering a cancer treatment.

37.-38. (canceled)

39. A method of determining the efficacy of treatment of cancer in a subject, comprising
i) determining the expression of PMEPA-1 on a tumor vascular endothelial cell after administering a cancer treatment, wherein increased expression of PMEPA-1 as compared to a control level is indicative of the presence or progression of the cancer;
ii) determining the expression of PMEPA-1 after administration of the cancer treatment, wherein decreased expression of PMEPA-1 as compared to a control level is indicative of effective cancer treatment,
optionally, wherein the control level is the expression of PMEPA-1 on the tumor vascular endothelial cell prior to administering the cancer treatment.

40. (canceled)

41. The method of claim 39, further comprising the step of administering the cancer treatment, optionally wherein the cancer treatment is a composition comprising
1) an antibody or antigen binding fragment thereof, which specifically binds to Prostate Transmembrane Protein, Androgen Induced 1 (PMEPA-1), and
2) an agent that (a) induces cell death, or (b) induces an inflammatory response to a tumor vascular endothelial cell in which the expression of PMEPA-1 is upregulated as compared to expression in a non-tumor vascular endothelial cell control.

42. (canceled)

43. A composition comprising the antibody or antigen binding fragment thereof of claim 1 associated with a detectable marker, optionally, wherein the detectable marker is selected from the group consisting of fluorescent labels, phosphorescent labels, chemiluminescent labels or bioluminescent labels, radio-isotopes, metals, metals chelates or metallic cations, chromophores and enzymes.

44. (canceled)

45. A medical imaging method comprising (i) administering the composition of claim 43, and (ii) detecting the antibody or antigen binding fragment thereof in the body of the patient.

46.-47. (canceled)

48. The method of claim 21, wherein:
(a) the tumor vascular endothelial cell is a venular cell;
(b) PMEPA-1 is not expressed in non-tumor vascular endothelial cells, wherein PMEPA-1 is expressed at higher levels in tumor vascular endothelial cells as compared to expression in non-tumor vascular endothelial cells, or the expression of PMEPA-1 in tumor vascular endothelial cells is a variant of PMEPA-1 expressed in non-tumor vascular endothelial cells; and/or
(c) PMEPA-1 is expressed at least 1.5-fold, at least 2-fold, at least 2.5-fold, at least 3-fold, at least 3.5-fold, at least 4-fold, at least 4.5-fold, or at least 5-fold more in tumor vascular endothelial cells as compared to expression in non-tumor vascular endothelial cells.

49.-69. (canceled)

70. The method of claim 21, wherein the cancer is
(i) a non-immunogenic cancer;
(ii) a hematological cancer;
(iii) a solid tumor;
(iv) selected from the group consisting of melanoma, pancreatic cancer, and colorectal cancer; or
(v) breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

71.-72. (canceled)
